(19) United States
(12) Patent Application Publication
(10) Pub. No.: US 2024/0074701 A1
(43) Pub. Date: Mar. 7, 2024

(54) WEARABLE CONTINUOUS EMERGENCY MEDICAL MONITORING SYSTEM

(71) Applicant: AYUDA MEDICAL, LLC, Normandy Park, WA (US)

(72) Inventors: Michael Hite, Normandy Park, WA (US); Desislava Hite, Normandy Park, WA (US)

(21) Appl. No.: 18/307,787

(22) Filed: Apr. 26, 2023

Related U.S. Application Data
(63) Continuation-in-part of application No. PCT/US2021/056701, filed on Oct. 26, 2021.
(60) Provisional application No. 63/113,078, filed on Nov. 12, 2020.
(60) Provisional application No. 63/106,804, filed on Oct. 28, 2020.

(51) Int. Cl. A61B 5/00; A61B 5/1455

(52) U.S. Cl. CPC A61B 5/6805; A61B 5/14552; A61B 5/4839; A61B 5/7285; A61B 5/746; A61B 5/747; A61B 5/11

(57) ABSTRACT

What is described is wearable device for a user comprising: at least one motion-state sensor; at least one oximetry sensor; an alarm module configured for transmitting an emergency signal; and a device control logic comprising firmware or embedded software, wherein the motion-state sensor activates the device control logic upon sensing a no-motion or low-motion state of the user, and the oximetry sensor activates the device control logic during a low-oxygenation or low-respiration state of the user, in which the wearable device continuously monitors and detects a respiratory emergency of the user. Also described is a system comprising the wearable device to communicate an emergency alert to appropriate responders, and a method of using the wearable device for a person at risk of respiratory failure.

### CROSS-REFERENCE TO RELATED APPLICATIONS

This application is a continuation-in-part application of International Application Number PCT/US2021/056701, filed on Oct. 26, 2021, which claims the benefit of U.S. provisional patent application Ser. No. 63/011,198, filed on Oct. 27, 2020, and 63/075,720, filed on Nov. 12, 2020, the contents of which are herein incorporated by reference in their entirety.

### BACKGROUND

Life-threatening changes in oxygenation or respiration are emergency medical events in which severe morbidity or mortality may occur if no intervention is performed. Patients are at highest risk when experiencing unexpected life-threatening changes in oxygenation or respiration outside of the hospital, in the at-home or ambulatory (outside-of-home) environments.

In many cases patients experiencing emergency medical conditions are incapacitated (i.e., loss of consciousness due to opioid overdose or acutely constricted airways during asthma or allergy events) and are not capable of self-treatment (i.e., self-rescue with a pharmaceutical medication, or calling 9-1-1), requiring immediate intervention, often within a few minutes, by a 3rd party such as a physician or nurse, caregiver, bystander, emergency medical responder, to prevent death or permanent damage due to hypoxia.

However, currently there are few wearable medical monitors to continuously monitor patients in the at-home or ambulatory environment that alarm or alert 3^{rd} parties to potentially life-threatening emergency medical events. Most wearable devices for vital sign monitoring are focused on management of chronic conditions, such as diabetes and hypertension, with biometric data primarily used for the purpose of observing patient response over time to new health behaviors or changes in medication regimens. Most wearable vital sign monitors will record out-of-specification readings (for providers to review at the next patient visit), but do not serve as real-time alert systems. Most real-time alert systems are designed to be engaged by the user if they experience or self-diagnose a medical event (i.e., medical alert button that is impossible for users suffering from potentially life-threatening respiratory decline. Most real-time alert systems are designed to alert others to movement-based events (i.e., fall detection), but do not include vital sign monitoring necessary to diagnose respiratory decline.

Wearable devices most often utilize periodic sampling of vital signs (i.e., ECG probes to collect data on cardiac rhythm and function; glucose monitors to collect data on blood glucose; pulse oximeters to collect data on oxygenation) to monitor chronic medical conditions, or relatively small, slow changes in respiration and oxygenation over time, but are generally not intended as acute care monitors. There is an unmet need for acute respiratory and oxygenation monitors for the at-home and ambulatory (outside of home) environments.

Patients with certain medical conditions or diseases may benefit from close medical monitoring to identify sudden changes in risk biomarkers, oxygenation or respiration that indicate potential acute, unanticipated life-threatening medical emergency.

One such medical condition is opioid or substance use disorders in which patients is monitored for opioid- or pharmacologically induced respiratory depression. More than two million people in the United States are estimated to have opioid use disorder (OUD) and more than 60,000 people in the United States died from opioid overdose in 2020.

Another such medical condition is epilepsy or other seizure disorders in which patients is monitored for acute respiratory or oxygenation failure during or after a seizure event, often referred to as Sudden Unexplained Death in Epilepsy Patients (SUDEP). More than 1.3 million people in the United States are estimated to have epilepsy or other seizure disorder and more than one-third of all epilepsy patients continue to have seizures on a regular basis, despite medication and other therapies.

Other medical conditions include acute events caused by exposure to environmental agents, such as severe food allergies (i.e., peanuts) or severe reactions to poisons or toxins (i.e., bee venom, organophosphate pesticides or nerve agents), which may trigger life-threatening respiratory symptoms.

Many such patients require continuous monitoring (i.e., vital sign or medical risk-state information captured at least once per minute) due to their risk of acute, unexpected respiratory depression or oxygenation decompensation. Monitoring risk biomarkers, in addition to oxygenation or respiration directly, may enable earlier detection and intervention to prevent life-threatening events from becoming fatal.

Such risk biomarkers include motion state (i.e., no/low motion state in opioid overdose), perspiration (i.e., high electrodermal activity state in allergic reaction), convulsions (i.e., seizure), temperature (i.e., fever or hypothermia), and response (i.e., failure to cancel alarm) which may indicate patient is experiencing a potentially life-threatening emergency medical event. In many situations, continuous operation of risk biomarker sensors is also more energy-efficient than continuous monitoring of oxygenation or respiration directly.

Continuous emergency medical monitoring in the at-home and ambulatory environments also requires the monitoring equipment be compatible with patient activities of daily living (ADL), such as sitting, standing, walking, driving, cooking, eating, bathing, sleeping, watching media, and communicating.

To facilitate compliance with continuous use in the at-home and ambulatory environments, monitoring equipment should be comfortable to wear (i.e., light, non-constricting), power efficient (charge >24 h), cleanable (i.e., alcohol wipes, soap, and water) and respect patient desire for privacy and data protection (i.e., monitoring equipment should be discreet and data secure).

If life-threatening changes in oxygenation, respiration or risk biomarkers occur, patients would benefit from a local alarm to identify the emergency medical event to bystanders (i.e., family, friends, caregivers, passersby) so they can provide medical aid or call for emergency medical assistance.

Patients would also benefit from a wireless alert to reach out via phone or text message to notify them of the emergency medical event and summon help from pre-established contacts (i.e., friends, family, healthcare providers).

Additionally, some patients may require bystander intervention to administer medications to reverse the emergency medical event (i.e., naloxone for opioid overdose, benzodiazepines for seizure). Patients would benefit from bystanders being informed of the need for medical intervention and instructions on how to administer medications.

Patients treated in hospital or clinic settings for medical conditions that may result in rapid and unexpected respiratory decline is monitored remotely using a combination of wearable devices, network access devices (i.e., wi-fi access points), and a central monitoring system (i.e., nursing station). Patients discharged into at-home or residential environments with medical conditions that may still result in rapid and unexpected respiratory decline need to be similarly monitored as in a hospital setting, but few continuous monitoring wearables can detect acute changes in respiration and oxygenation.

Continuous respiration and oxygenation monitoring devices which measure vital signs at least once per minute are needed for patients who are hospitalized or who are at risk of experiencing unexpected acute distress, but most continuous monitoring technologies are cumbersome and interfere with ADLs. Notably, most continuous monitoring devices are size-limited due to the significant power requirements of monitoring sensors and corresponding size of battery. There is a significant need for smaller and more power efficient continuous monitoring devices compatible with ADLs.

Current monitoring devices focus largely on patients with chronic cardiac conditions, and thus use ECG sensors as their primary diagnostic tool for continuous monitoring of pulse rate (PR) and respiration rate (RR). Unfortunately changes in ECG measurements and PR is unreliable indicators of acute centrally mediated apnea or hypopnea, notably in cases of pharmacologically induced respiratory depression (such as seen in opioid overdose), and exacerbations of underlying medical conditions such epilepsy or other seizure disorders.

oximetry-based systems, rather than ECG-based systems, are needed to accurately detect acute changes in respiration and oxygenation. Current wearable vital sign monitoring systems that utilize oximetry sensors to measure patient oxygenation (SpO2) are largely limited to spot check (periodic) measurements for oxygenation, mostly through use of a finger-tip pulse-oximeter, but unfortunately, rapid and unexpected changes in patient oxygenation may not be identified if they occur in the intervals between spot checks.

There is no continuous respiratory depression monitor currently available that uses motion state and oximetry as its primary diagnostic measurements. There is also no continuous respiratory depression monitor that utilizes a lack of response (i.e., patient lack of motion to stimulation; failure to cancel alarm) as an indication of patient unresponsiveness or unconsciousness.

One of the challenges of continuous respiratory and oxygenation monitoring is prevention of “false positive” alarms due to sensor faults, (i.e., failure of skin-sensor contacts, probe “liftoff” light artifacts, or motion artifacts that cause the sensor to fail to capture data) notably from sensor probes located on the wrists and fingertips. There is a need for a high-fidelity continuous monitor with a low-rate of “false positive” alarms, preferably for wear on a location other than the high-artifact locations of the wrist or fingertips.

Most wearable vital sign monitoring devices utilize only a single analytical sensor for each type of data (i.e., a single oximetry probe). The use of a single sensor can result in more frequent “false positive” alarms due to the lack of any other data to suggest the User is not having a medical event. There are currently no approved medical devices utilizing multiple independent oximetry sensors to reduce the incidence of “false positive” alarms.

Most oximetry devices use a single photoplethysmography (PPG) sensor, even if the probe is comprised of multiple types of photo emitters and receivers in different wavelengths (i.e., green, red and infrared probes). A single PPG sensor strictly limits the performance range of the photo emitter/detector pair, and the single PPG sensor cannot compensate for lighter or darker skin tones. There is a need for a multi-PPG sensor reflective probe for wearable devices that would allow a larger dynamic range.

Current wearable vital sign monitoring devices most often rely upon WLAN (Wireless Local Area Network) communication to transmit vital sign data to a remote server on the internet (a “cloud server”). Such WLAN communication relics upon a local network for access to the internet, such as home or facility internet connection using a Wi-fi router or “Wi-fi base-station;” the wearable device transmits signal to the “Wi-fi base station,” where it is then transmitted to a remote server on the internet. Base-station monitoring systems require the wearable device (and thus the user) stay within a limited-range of the base-station (i.e., within 50 feet). There is currently no continuous vital-sign monitoring device, even for chronic conditions, that is capable of independent wireless communication outside the range of the base-station, (i.e., outside the home).

Therefore, a need exists for an accurate (including low incidence of false-positive alarms) remote monitoring platform for acute respiratory emergencies in the ambulatory and outpatient environments that is capable of alarming and sending real-time wireless alerts for emergency medical intervention and follow-up.

### SUMMARY OF INVENTION

A wearable Emergency Medical Monitoring System is described, including: a wearable device capable of continuous monitoring of patient for a high-risk state using a non-oximetry biosensor, detecting life-threatening changes in respiration and oxygenation capable, and activating an alarm system; and a software application located on a

A wearable Emergency Medical device is described, including: one or more oximetry sensors and probes; at least one non-oximetry biosensor; an alarm system; a device operation system; and device control logic to activate alarm system when non-oximetry biosensors indicate a high-risk state, or when oximetry sensors indicate a high-risk oxygenation or respiration state, or a combination thereof.

A method is disclosed for preventing respiratory decline from becoming fatal by engaging patient and bystanders with alarms, pre-selected contacts with wireless emergency alerts (phone and text notifications), or emergency medical services, volunteers or other 3^{rd} parties who can render immediate medical assistance.

In one aspect, the system includes a communication module on a wearable device capable of wireless transmission of an electronic signal to another communicably coupled device.

In one aspect, the system includes a communication module on a wearable device capable of wireless transmission of a radio frequency electronic signal to another communicably coupled device.

In one embodiment, the system includes a Bluetooth radio communication module on a wearable device capable of wireless communication via Bluetooth.

In one embodiment, the system includes a WLAN radio communication module on a wearable device capable of wireless communication via WLAN.

In one embodiment, the system includes a cellular radio communication module on a wearable device capable of wireless communication via cellular.

In one embodiment, the system includes a communication module on a wearable device capable of wireless transmission of an electronic signal to a communicably coupled remote monitoring system.

In one embodiment, the system includes software on the wearable device to engage communications module upon identification of a potentially life-threatening respiratory event using information from a combination of oximetry and non-oximetry sensors.

In one embodiment, the system includes software located on a remote device to receive emergency alert signal from wearable device upon identification of a potentially life-threatening respiratory event using information from a combination of oximetry and non-oximetry sensors.

In one embodiment, the system includes software located on a remote device capable of sending an emergency message to pre-designated contacts.

In one embodiment, the system includes software located on a remote device capable of sending an emergency message, alert or electronic communication to a remote monitoring system

In one embodiment, the system includes software located on a remote device capable of sending an emergency message, alert or electronic communication to emergency medical services (i.e., 9-1-1).

In one embodiment, the system includes a wearable device with Control Logic to activate oximetry sensors after receiving information from remote non-oximetry sensors indicating user is in a high-risk state.

In one embodiment, the system includes a wearable device with Control Logic to activate oximetry sensors after receiving information from remote non-oximetry sensors indicating user is in a hypopneic or apneic state.

In one embodiment, the system includes a wearable device with Control Logic to activate oximetry sensors after receiving information from remote non-oximetry sensors indicating user is in a high-risk motion state (i.e., fallen, unmoving)

In one embodiment, the system includes a wearable drug-delivery device, including autoinjector, bolus-injector, on-body injector, patch, or patch-pump communicably coupled to the wearable device.

In one embodiment, the device includes Control Logic to activate a wearable drug-delivery device, including autoinjector, bolus-injector, on-body injector, patch, or patch-pump.

In one embodiment, the device includes Control Logic on a remote device to activate a wearable drug-delivery device, including autoinjector, bolus-injector, on-body injector, patch, or patch-pump, after receiving information from wearable device that user in experiencing an emergency medical event.

In one embodiment, the wearable device oximetry sensors include at-least two independent oximetry sensors.

In one embodiment, the wearable device oximetry sensors include at-least two oximetry probes per sensor.

In one embodiment, the wearable device oximetry sensors include at-least one photoplethysmography (PPG) sensor.

In one embodiment, the wearable device oximetry sensors include at-least two PPG sensors.

In one embodiment, the wearable device oximetry sensors include PPG probes, wherein each probe contains at-least two photo emitter-receiver pairs in the red spectrum (620-750 nm) and at-least two in the infrared spectrum (780 nm-1 mm).

In another embodiment, the wearable device oximetry sensors include PPG probes, wherein each probe contains at-least one photo emitter-receiver pair in the green spectrum (620-750 nm), at-least two photo emitter-receiver pairs in the red spectrum (620-750 nm), and at-least two in the infrared spectrum (780 nm-1 mm).

In another embodiment, the wearable device PPG probes include at-least one PPG optical photo emitter in the red spectrum is oriented at an approximately 90-degree angle from at-least one other photo receiver in the red spectrum, and at-least one PPG optical photo emitter in the infrared spectrum is oriented at an approximately 90-degree angle from at-least one other photo receiver in the infrared spectrum.

In another embodiment, the wearable device PPG probes include at least one PPG optical photo emitter in the red spectrum is oriented in an approximately 90-degree angle from at least one other photo emitter in the red spectrum, and at least one PPG optical photo emitter in the infrared spectrum is oriented in an approximately 90-degree angle from at least one other photo emitter in the infrared spectrum.

In another embodiment, the wearable device PPG probes include at-least two PPG optical photo emitters in the red spectrum are oriented at an approximately 90-degree angle from at-least one other photo receiver in the red spectrum, and at-least two PPG optical photo emitters in the infrared spectrum are oriented at an approximately 90-degree angle from at-least one other photo receiver in the infrared spectrum.

In another embodiment, the wearable device PPG probes include at least two PPG optical photo emitters in the red spectrum, oriented in an approximately 180-degree angle from one another, and at least two PPG optical photo emitters in the infrared spectrum, oriented in an approximately 180-degree angle from one another.

In one embodiment, the wearable device oximetry sensors include PPG probes, wherein the optical distance between the photo emitter and receivers is 6-10 mm.

In one embodiment, the wearable device oximetry sensors include PPG probes, wherein the optical distance between the photo emitter and receiver is 9-10 mm.

In one embodiment, the wearable device oximetry sensors include reflective (vs transmissive) PPG probes.

In one embodiment, the wearable device oximetry sensors include at-least one near-infrared spectroscopy (NIRS) sensor.

In one embodiment, the wearable device non-oximetry sensors include at-least one motion-state sensor.

In another embodiment, the wearable device non-oximetry sensors include at-least two motion-state sensors.

In one embodiment, the wearable device motion-state sensors include at-least one accelerometers.

In one embodiment, the wearable device motion-state sensors include at least-one gyroscope.

In one embodiment, the wearable device motion-state sensors include at-least one magnetometer.

In one embodiment, the wearable device motion-state sensors include at-least one surface electromyograph sensor.

In one embodiment, the wearable device motion-state sensors include at-least two surface electromyograph sensors.

In one embodiment, the wearable device motion-state sensors include at-least one surface electromyograph sensor on the pectoralis major.

In one embodiment, the wearable device motion-state sensors include at-least one surface electromyograph sensor on the trapezius.

In one embodiment, the wearable device surface electromyograph sensors include conductive fabric.

In one embodiment, the wearable device surface ECG sensors include conductive fabric.

In one embodiment, the wearable device non-oximetry sensors include a heart-rate sensor (i.e., single-lead ECG, green-light PPG).

In one embodiment, the wearable device non-oximetry sensors include at-least one glucose-monitor.

In one embodiment, the wearable device non-oximetry sensors include at-least one temperature sensors (i.e., contact thermometers).

In one embodiment, the wearable device non-oximetry sensors include at-least one electrodermal activity sensor.

In one embodiment, the wearable device non-oximetry sensors include at-least one noninvasive monitor for a chemical analyte, metabolite, or combination thereof.

In one embodiment, the wearable device non-oximetry sensors include a sensor located on a remote device (i.e., mobile device, home device, auto device).

In one embodiment, the wearable device is configured for somatic locations.

In one embodiment, the wearable device is configured for location on the proximal limb (i.e., upper arm or upper leg).

In one embodiment, the wearable device is configured for location on the upper torso.

In one embodiment, the wearable device is configured for location as an on-ear device.

In one embodiment, the wearable device is configured for location as an in-ear device.

In one embodiment, the wearable device is configured for bilateral wear wherein oximetry probes are located over comparable or functionally similar target tissues when worn by the user on either right or left body locations

In one embodiment, the wearable device is configured for bilateral wear wherein non-oximetry probes are located over comparable or functionally similar target tissues when worn by the user on either right or left body locations

In one embodiment, one or more of the wearable device major functional components are located on discrete physical segments of the device and are in communication with one another.

In one embodiment, one or more of the wearable device major functional components are located on discrete physical segments of the device and are in communication with one another using flexible circuit board substrate.

In another embodiment, one or more of the wearable device major functional components are located on discrete portions of the device and are in communication with one another using wireless communication.

In one embodiment, the wearable device functional components distributed along a series of segments arrayed in an Arc, around the circumference of the limb or torso measuring 90-180 degrees,

In one embodiment, the wearable device functional components distributed along a series of segments arrayed in an Arc, along or in-line with the limb or torso measuring 45-120 degrees.

In one embodiment, the wearable device functional components distributed along a series of segments configured in a linear orientation, and wherein the oximetry probes are located on distal segments of the device.

In one embodiment, the wearable device functional components distributed along a series of segments configured in a linear orientation, and wherein the non-oximetry probes are located on distal segments of the device.

In one embodiment, the wearable device functional components distributed along a series of segments configured in a linear orientation, and wherein the oximetry probes and non-oximetry probes are both located on distal segments of the device.

In one embodiment, the wearable device oximetry sensor probes are in contact with both medial and lateral sides, or anterior and posterior sides, in relation to user limb or torso.

A method is disclosed for improving the proportion of interpretable signals and enabling signal capture from oximetry sensors of the wearable device, regardless of user body position, by locating oximetry probes on medial and lateral sides, or anterior and posterior sides, relative to limb or torso

A method is disclosed for improving the proportion of interpretable signals and enabling signal capture from oximetry sensors of the wearable device, regardless of user body position, by adjustment of skin-sensor pressure through use of force-adjustment mechanisms, including motors, springs, cables, or elastomeric material.

In one embodiment, the wearable device non-oximetry sensor probes are in contact with both medial and lateral sides, or anterior and posterior sides, in relation to user limb or torso.

In one embodiment, the wearable device motion-state sensor probes are in contact with both medial and lateral sides, or anterior and posterior sides, in relation to user limb or torso.

In one embodiment, the wearable device segments are mounted on an underlayment of malleable metal substrate such as aluminum, or elastomeric 3D printed substrate.

In one embodiment, the wearable device segments containing the optical probes include hinge, pivot, or rotation mechanisms to improve optical probe contact with user skin.

In one embodiment, the wearable device segments containing optical probes hinge, pivot or rotation mechanisms are manually adjusted.

In one embodiment, the wearable device segments containing optical probes hinge, pivot or rotation mechanisms are automatically or passively adjusted (i.e., spring(s), motor, elastomeric material).

In one embodiment, the wearable device segments include spring, hinge, pivot, or rotate mechanisms to improve optical probe contact with user skin.

In one embodiment, the wearable device segments containing the optical probes include force-adjustment or sensor/skin-pressure adjustment mechanisms to improve contact with the user skin.

In one embodiment, the wearable device segments containing the optical probes force-adjustment or sensor/skin-pressure adjustment mechanisms are manually adjusted.

In one embodiment, the wearable device segments containing the optical probes force-adjustment or sensor/skin-pressure adjustment mechanisms are automatically or passively adjusted (i.e., spring(s), motor, elastomeric material).

In one embodiment, the wearable device includes a physical interface separable from the electronic components of the device, between the electronic components of the device and the skin of the user (i.e., a platform, underlayment, superstructure, frame or garment), that provides anatomical shape to electronic device components and enables device segments containing the oximetry probes to be in contact with the user's skin, and the device control logic, speaker and battery are located on segments not in contact with the user's skin.

In one embodiment, the wearable device is a thoracic wearable containing an electronics underlayment, separable from the wearable garment.

In one embodiment, the wearable device is a thoracic wearable containing a multi-layer garment, comprising an outer layer, an inner layer, and an electronics underlayment, separable from the wearable garment.

In one embodiment, the wearable device includes a physical interface enables different zones of compression, wherein device segments containing the oximetry probes are compressed at a comparatively greater force than device segments containing device control logic, speaker or battery.

In one embodiment, the wearable device continuously operates a comparatively lower-power non-oximetry sensor, and only activates comparatively higher-power oximetry sensors following indication from the non-oximetry sensor of a high-risk state.

In one method for continuous monitoring, the wearable device continuously operates a comparatively lower-power non-oximetry sensor, and only activates comparatively higher-power oximetry sensors following indication from the non-oximetry sensor of a high-risk state.

In one embodiment, the wearable device includes device control logic to operate device in two states; resting state and alarm state.

In one embodiment, the wearable device includes device control logic to switch between resting and alarm states, using information from non-oximetry biosensors to indicate a high-risk state or information from oximetry sensors indicating high-risk oxygenation or respiration states.

In one embodiment, the wearable device includes device control logic to operate resting state using information from non-oximetry biosensors to indicate a low-risk state.

In one embodiment, the wearable device includes device control logic to operate in an alarm state using information from non-oximetry biosensors to indicate a high-risk state.

In one embodiment, the wearable device includes device control logic to operate in a resting state using information from oximetry sensors to indicate low-risk oxygenation or respiration states.

In one embodiment, the wearable device includes device control logic to operate in an alarm state using information from oximetry sensors to indicate high-risk oxygenation or respiration states.

In one embodiment, the wearable device includes device control logic to operate in a resting state using information from motion-state sensors to indicate a low-risk state.

In one embodiment, the wearable device includes device control logic to operate in an alarm state using information from motion-state sensors to indicate a high-risk state.

In one embodiment, the wearable device non-oximetry sensors include at-least one motion-state sensor and device control logic to activate alarm system when motion-state sensor indicate a high-risk state, or when oximetry sensors indicate a high-risk oxygenation or respiration state, or a combination thereof.

In one embodiment, the wearable device non-oximetry sensors include at-least one motion-state sensor and device control logic to activate alarm system when motion-state sensor indicate a high-risk no-motion/low-motion state, or when oximetry sensors indicate a high-risk oxygenation or respiration state, or a combination thereof.

In one embodiment, the wearable device non-oximetry sensors include at-least one motion-state sensor and device control logic to activate alarm system when motion-state sensor indicate a high-risk seizure/high-motion state, or when oximetry sensors indicate a high-risk oxygenation or respiration state, or a combination thereof.

In one embodiment, the wearable device non-oximetry sensors include at-least one temperature-state sensor and device control logic to activate alarm system when temperature-state sensor indicate a high-risk state, or when oximetry sensors indicate a high-risk oxygenation or respiration state, or a combination thereof.

In one embodiment, the wearable device non-oximetry sensors include at-least one temperature-state sensor and device control logic to activate alarm system when temperature-state sensor indicate a high-risk high-temperature state, or when oximetry sensors indicate a high-risk oxygenation or respiration state, or a combination thereof.

In one embodiment, the wearable device non-oximetry sensors include at-least one temperature-state sensor and device control logic to activate alarm system when temperature-state sensor indicate a high-risk low-temperature state, or when oximetry sensors indicate a high-risk oxygenation or respiration state, or a combination thereof.

In one embodiment, the wearable device non-oximetry sensors include at-least one glucose-state sensor and device control logic to activate alarm system when glucose-state sensor indicate a high-risk glucose state, or when oximetry sensors indicate a high-risk oxygenation or respiration state, or a combination thereof.

In one embodiment, the wearable device non-oximetry sensors include at-least one glucose-state sensor and device control logic to activate alarm system when glucose-state sensor indicate a high-risk high-glucose state, or when oximetry sensors indicate a high-risk oxygenation or respiration state, or a combination thereof.

In one embodiment, the wearable device non-oximetry sensors include at-least one glucose-state sensor and device control logic to activate alarm system when glucose-state sensor indicate a high-risk low-glucose state, or when oximetry sensors indicate a high-risk oxygenation or respiration state, or a combination thereof.

In one embodiment, the device includes Control Logic to activate oximetry sensors after receiving information from non-oximetry sensors that user is in a high-risk state.

In one embodiment, the device includes Control Logic to activate oximetry sensors after receiving information from Motion-state sensors that user in a high-risk state.

In one embodiment, the device includes Control Logic to activate oximetry sensors after receiving information from Motion-state sensors that user in a high-risk No-Motion or Low Motion state.

In one embodiment, the device includes Control Logic to activate oximetry sensors after receiving information from Motion-state sensors that user in a high-risk High Motion or Seizure state.

In one embodiment, the device includes an alarm module containing at least two alarms, including a tone alarm, audio speaker and haptic alarm.

In one embodiment, the device alarm system includes an audio tone alarm.

In one embodiment, the device alarm system includes an escalating audio tone alarm, up to 85 dB at 10 ft.

In one embodiment, the device alarm system includes an escalating audio tone alarm, to 85-105 dB at 10 ft.

In one embodiment, the device alarm system includes an escalating audio tone alarm, to over 105 dB at 10 ft.

In one embodiment, the device alarm system includes an audio speaker capable of delivering a voice message.

In some embodiments, the alarm system includes an audio speaker capable of generating a voice message and a tone alarm.

In one embodiment, the device alarm system includes an audio speaker capable of delivering a voice message to bystanders at high volume (>80 dB at 10 ft, unobstructed).

In one embodiment, the device alarm system includes an audio speaker capable of delivering a voice message to engage user (i.e., “Are you ok?”) or prompt a user response (i.e., “Press cancel to stop alarm.”).

In one embodiment, the device alarm system includes an audio speaker capable of delivering a voice message to bystanders to identify the emergency medical event (i.e., “This is a medical emergency”)

In one embodiment, the device alarm system includes an audio speaker capable of delivering a voice message to bystanders to provide information regarding the emergency medical event.

In one embodiment, the device alarm system includes an audio speaker capable of delivering a voice message to bystanders to request help or call emergency medical assistance (9-1-1).

In one embodiment, the device alarm system includes an audio speaker capable of delivering a voice message to bystanders with instructions to perform rescue breathing or CPR.

In one embodiment, the device alarm system includes an audio speaker capable of delivering a voice message to bystanders with instructions to administer a rescue medication.

In one embodiment, the device alarm system includes an audio speaker capable of delivering a voice message to bystanders with instructions on information how to administer a rescue medication.

In one embodiment, the device alarm system includes a haptic alarm (vibratory alert).

In some embodiments, the alarm system includes a speaker capable of generating a voice message and a separate audio component capable of generating a tone alarm.

In one embodiment, the device alarm system includes a haptic alarm (vibratory alert) and vibrating micro-motor powered by more than 2V.

In one embodiment, the device alarm system includes a haptic alarm (vibratory alert) and vibrating micro-motor generating a vibration pattern of 120-180 Hz.

In one embodiment, the device alarm system includes a haptic alarm (vibratory alert) and vibrating micro-motor generating a vibration pattern of more than 1801 Hz.

In one embodiment, the device alarm system includes a haptic alarm (vibratory alert) and vibrating micro-motor generating a vibration pattern with gap lengths of less than 3M) milli-seconds between vibrations.

In one embodiment, the device alarm system includes a haptic alarm (vibratory alert) and vibrating micro-motor generating a vibration pattern with gap lengths of 300-600 milli-seconds between vibrations.

A method is disclosed for preventing respiratory decline from becoming fatal by using a wearable device that will engage alarm module to engage user's and bystanders' attention and response upon receiving information of a high-risk state from non-oximetry biosensors, or high-risk oxygenation or respiration states from oximetry sensors.

A method is disclosed for preventing respiratory decline from becoming fatal by using a wearable device that will engage audio alarm module to engage user's and bystanders' attention and response.

A method is disclosed for preventing respiratory decline from becoming fatal by using a wearable device that will engage audio alarm module to verbally identify emergency event to bystanders.

A method is disclosed for preventing respiratory decline from becoming fatal by using a wearable device that will engage audio alarm module to verbally provide information regarding emergency event to bystanders.

A method is disclosed for preventing respiratory decline from becoming fatal by using a wearable device that will engage audio alarm module to verbally request bystanders respond to emergency medical event or call for emergency medical assistance.

A method is disclosed for preventing respiratory decline from becoming fatal by using a wearable device that will engage audio alarm module to verbally instruct bystanders respond to emergency medical event or call for emergency medical assistance.

A method is disclosed for preventing respiratory decline from becoming fatal by using a wearable device that will engage audio alarm module to verbally instruct bystanders to administer rescue medication.

A method is disclosed for preventing respiratory decline from becoming fatal by using a wearable device that will engage audio alarm module to verbally instruct bystanders how to administer rescue medication.

A method is disclosed for preventing respiratory decline from becoming fatal by using a wearable device that will stimulate user to respond and improve respiration or oxygenation.

A method is disclosed for preventing respiratory decline from becoming fatal by using a wearable device with a haptic alarm (vibratory alert) that will stimulate user to respond and improve respiration or oxygenation.

A method is disclosed for preventing respiratory decline from becoming fatal by using a wearable device with an audible alarm that will stimulate user to respond and improve respiration or oxygenation.

A method is disclosed for preventing respiratory decline from becoming fatal by using a wearable device with an electrical or thermal stimulation mechanism that will stimulate the user to respond and improve respiration or oxygenation.

A method is disclosed for preventing respiratory decline from becoming fatal by using a wearable device with an voice speaker that will stimulate user to respond and improve respiration or oxygenation using verbal messages (i.e., “Are you ok?, “Wake up!”).

A method is disclosed for preventing respiratory decline from becoming fatal by using a wearable device that will engage communications module to summon pre-designated contacts either directly or through a communicably coupled remote device.

A method is disclosed for preventing respiratory decline from becoming fatal by using a wearable device that will engage communications module to summon emergency medical assistance (9-1-1) either directly or through a communicably coupled remote device.

A method is disclosed for preventing pharmacologically induced respiratory depression from becoming fatal through use of a wearable Emergency Medical Monitoring System.

A method is disclosed for preventing pharmacologically induced respiratory depression from becoming fatal through use of a wearable Emergency Medical Monitoring System by patients at high-risk for opioid overdose, including patients diagnosed with opioid- and substance-use disorders, patients taking opioids or other medications to depress respiratory drive, and patients with prior history of overdose.

A method is disclosed for preventing acute post-ictal respiratory depression from becoming fatal through use of a wearable Emergency Medical Monitoring System.

A method is disclosed for preventing acute post-ictal respiratory depression from becoming fatal through use of a wearable Emergency Medical Monitoring System by patients diagnosed with epilepsy or other seizure disorder, patients with uncontrolled seizure, and patients with prior history of seizure.

### BRIEF DESCRIPTION OF THE DRAWINGS

To easily identify the discussion of any element or act, the most significant digit or digits in a reference number refer to the FIG. number in which that element is first introduced. The wearable Emergency Medical Monitoring System 100 comprises a wearable oximetry device, Mobile Application and Remote device or Remote Monitoring System.

FIG. 1A illustrates a wearable device 101 in accordance with one embodiment of wearable System 100.

FIG. 1B illustrates a wearable oximetry device 101 in accordance with one embodiment of wearable System 100.

FIG. 2A illustrates a Method 200 of operation of wearable System 100 in accordance with one embodiment.

FIG. 2B illustrates a Method 201 of operation of wearable System 100 in accordance with one embodiment.

FIG. 3A illustrates configurations of the wearable device 301 in accordance with some embodiments.

FIG. 3B illustrates configurations of the wearable device and platform 360 in accordance with some embodiments.

FIG. 3C illustrates configurations of the wearable device and platform 360 in accordance with some embodiments.

FIG. 3D illustrates configurations of the wearable device and platform 360 in accordance with some embodiments.

FIG. 3E illustrates configurations of the wearable oximetry device platform and garment in accordance with some embodiments.

FIG. 3F illustrates one embodiment of wearable device 301, comprising at least two oximetry probes (331 and 351) and at least two non-oximetry biometric sensors (358 and 359) communicable coupled with device PCB 318, and device PCB 318 communicably coupled with power supply 338 and an alarm module containing both vibratory alert 323 and audio alarm 321. In this example, all device components are distributed across multiple segments in an approximately linear fashion.

FIG. 3G illustrates one embodiment of wearable device 301, comprising at least two oximetry probes (331 and 351) within a single housing, and at least two non-oximetry biometric sensors (358 and 359) communicable coupled with device PCB 318, and device PCB 318 communicably coupled with power supply 338 and an alarm module containing both vibratory alert 323 and audio alarm 321. In this example, all device components are distributed across multiple segments in an approximately linear fashion.

FIG. 4A illustrates configurations of the Bilateral Platform 400 of the wearable oximetry device 301 in accordance with some embodiments.

FIG. 4B illustrates configurations of the Bilateral Platform 400 of the wearable oximetry device 301 in accordance with some embodiments.

FIG. 4C illustrates configurations of the wearable device 301 in accordance with some embodiments.

FIG. 4D illustrates configurations of the wearable device 301 in accordance with some embodiments.

FIG. 5A illustrates an embodiment of a Digital Apparatus 500 to implement components and process steps of the system disclosed herein.

FIG. 5B illustrates an embodiment of the Communication Pathways 501 of wearable System 300 in accordance with some embodiments.

FIG. 5C illustrates an embodiment of the Communication Pathways 501 of wearable System 300 in accordance with some embodiments.

FIG. 6A illustrates configurations of wearable device platform 360 in accordance with some embodiments orienting components in relation to the Arc measured along the circumference or length of the body location.

FIG. 6B illustrates configurations of wearable oximetry device Platform 360 in accordance with some embodiments orienting components in relation to the arc measured along the circumference or length of the body location.

FIG. 7A illustrates Configurations of a wearable oximetry device 701 in accordance with some embodiments, orienting components in relation to the wearer's body and to one another.

FIG. 7B illustrates Configurations 700 of a wearable oximetry device 301 in accordance with some embodiments, orienting components in relation to the wearer's body and to one another.

FIG. g illustrates a wearable oximetry device 301 and wearable Garment 880 in accordance with some embodiments, suitable for use of the Upper Limb.

FIG. 9 illustrates wearable oximetry device Component Configurations in accordance with some embodiments utilizing flexible circuit substrate or wireless communication to transfer information between device components

FIG. 10 describes device Alarm Logic and Alert Logic, and Remote Monitoring System Alert Response Logic and Alert Follow-Up Logic for a Medical Event.

FIG. 11A describes device Alarm Logic and Alert Logic, and Remote Software Alert Response Logic and Alert Follow-Up Logic for the Medical Event of potential pharmacologically induced respiratory depression, in accordance with some embodiments.

FIG. 11B describes device Alarm Logic and Alert Logic, and Remote Software Alert Response Logic and Alert Follow-Up Logic for the Medical Event of potential pharmacologically induced respiratory depression, in accordance with some embodiments

FIG. 11C describes device Alarm Logic and Alert Logic, and Remote Monitoring System Alert Response Logic and Alert Follow-Up Logic for the Medical Event of seizure, in accordance with some embodiments.

FIG. 12 describes device Alarm Logic and Alert Logic, and Remote Monitoring System Alert Response Logic and Alert Follow-Up Logic for the Medical Event of seizure, in accordance with some embodiments.

### DETAILED DESCRIPTION

Unless defined otherwise, all technical and scientific terms used herein have the same meaning as commonly understood by one of ordinary skill in the art pertinent to the compositions and methods described. As used herein, the following terms and phrases have the meanings ascribed to them unless specified otherwise.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

Various logic functional operations disclosed herein are implemented in logic that is referred to using a noun or noun phrase reflecting said operation or function. For example, an association operation is carried out by an “associator” or “correlator”. Likewise, switching is carried out by a “switch”, selection by a “selector”, and so on.

As used herein the specification, “a” or “an” may mean one or more.

“Circuitry” in this context refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices disclosed herein, or a microprocessor configured by a computer program which at least partially carries out processes or devices disclosed herein), circuitry forming a memory device (e.g., forms of random access memory), or circuitry forming a communications device (e.g., a modem, communications switch, or optical-electrical equipment).

“Firmware” in this context refers to software logic embodied as processor-executable instructions stored in read-only memories or media.

“Hardware” in this context refers to logic embodied as analog or digital circuitry.

The term “intervene” or “intervention”, and the like, refers to and encompasses therapeutic or emergency rescue measures for a disease or disorder leading to clinically desirable or beneficial effect, including, but not limited to, alleviation or relief of one or more symptoms, regression, slowing or cessation of progression of emergency medical event, disease or disorder. Intervention can be evidenced as prevention of mortality, a decrease in severity of morbidity, decrease in severity of symptoms, frequency of symptoms, number of symptoms or frequency of relapse.

“Logic” in this context refers to machine memory circuits, non-transitory machine-readable media, and/or circuitry which by way of its material and/or material-energy configuration comprises control and/or procedural signals, and/or settings and values (such as resistance, impedance, capacitance, inductance, current/voltage ratings, etc.), that is applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

A “no-motion” state means physically still, not the absolute absence of any motion. A “no-motion” state is not a “low-motion” state as commonly understood by users of consumer health wearable devices—often meant as “not actively moving,” and breathing and communicating normally—a “no-motion” state would require physical stillness, breath-holding or shallow, slow, or imperceptible breathing, and no obvious communication. A “no-motion” state is a “very low-motion” state as measured via motion-state sensors (i.e., accelerometer, gyroscope). A “no-motion” state means no perceptible movement by a third-party observer. A “no-motion” state also means no clinically relevant movement, such as respiration.

A “non-responsive state” means unable to respond to one or more forms of stimuli, including audible, visual, and tactile. A “non-responsive state” may also mean a lack of response from stimuli, including a lack of motion.

“Software” in this context refers to logic implemented as processor-executable instructions in a machine memory (e.g., read/write volatile or nonvolatile memory or media).

“Remote Software Application” in this context refers to logic implemented as processor-executable instructions in a machine memory (e.g., read/write volatile or nonvolatile memory or media) not located on device 301.

The term “User” or “patient” shall refer to and encompass any user of the device.

For clarity of disclosure, and not by way of limitation, the detailed description of the invention is divided into the subsections which follow.

A wearable Emergency Medical Monitoring System is described, including: a wearable device capable of continuous monitoring of patient for a high-risk state using a non-oximetry biosensor, detecting life-threatening changes in respiration and oxygenation capable, and activating an alarm system; and a software application located on a

A wearable Emergency Medical device is described, including: one or more oximetry sensors and probes; at least one non-oximetry biosensor; an alarm system; a device operation system; and device control logic to activate alarm system when non-oximetry biosensors indicate a high-risk state, or when oximetry sensors indicate a high-risk oxygenation or respiration state, or a combination thereof.

A method is disclosed for preventing fatal respiratory decline by engaging patient and bystanders with alarms, pre-selected contacts with wireless emergency alerts (phone and text notifications), or emergency medical services, volunteers or other third parties who can render immediate medical assistance.

The device may also include a wearable device platform (patient-device physical interface) which is optionally located within a wearable garment.

The system may also include a remote monitoring system and a mobile application user interface. Additionally, the wearable device is in communication with a pharmaceutical delivery device.

Each oximetry sensor contains at least one oximetry signal processor and oximetry probes. Each oximetry sensor integrated with the device operation system or is a separate oximetry module. Such an oximetry module may contain a printed circuit board with one or more oximetry signal processors, power, data management and communication components, allowing the oximetry module to communicate to the device operation system.

The oximetry sensors and oximetry modules may be co-located with the oximetry probes, or may be located on a separate device component.

The oximetry sensors and oximetry modules may utilize signal processors and probes to support pulse-oximetry using photoplethysmographic (PPG) sensors, near-infrared spectroscopy (NIRS) sensors, or a combination of the two. PPG is defined as an instrument for measuring changes in light absorption and backscatter reflecting changes in blood volume. PPG sensors estimate arterial oxygen saturation (SpO2) by comparing red (620-750 nm) and infrared light (750-1000 nm) absorption and backscatter from arterial and venous blood. PPG sensors correlate the periodic changes in blood volume to the periodic rhythm of cardiac muscle contraction and circulatory system pulsation, allowing measurement of arterial blood (i.e., period of relatively higher volumes during the pulsatile phase) vs. venous blood (i.e., period of relatively lower blood volumes). NRS is defined as an instrument for measuring changes in light absorption and backscatter reflecting the ratio of oxygenated to dc-oxygenated hemoglobin present in tissues. NIRS sensors estimate regional or tissue oxygenation (rSO2) by comparing near-infrared light (700-900 nm) absorption and backscatter from target tissue.

The at-least one non-oximetry biosensor may include motion-state sensors, temperature sensors, electrodermal activity sensors, heart rate or pulse rate sensors, glucose sensors among others. The at-least one non-oximetry biosensor may include on-board sensor(s) contained in the device body, wirelessly linked biometric sensors, environmental sensors, and/or combinations thereof.

The at-least one non-oximetry biosensor may by a motion-state sensor, including an accelerometer, gyroscope, magnetometer, CMOS sensors (optical camera), surface electromyograph (sEMG), or a combination thereof.

In some configurations, the motion state sensor may also collect and monitor signals correlating to the device status, such as: device anatomic location on body and orientation.

In some configurations, the device control logic may also collect and monitor signals correlating to battery characteristics, communications module readiness and data upload status.

In some configurations, the system is connected to another device for the purpose of collecting additional patient data such as blood glucose data, physical location or mobility, detection of compounds within the environment, or a combination thereof, to determine whether a user is having an emergent medical event. For example, the system is connected to a continuous glucose monitor for the purpose of correlating vital sign data to glucose levels.

device operating system is configured for audible, visual, and tactile/vibratory alarms when device control logic determines sensor measurements are out of specified range.

device operating system contains a central processing unit (CPU), firmware and device operating software, control logic, memory, a power supply and charging mechanism. device may optionally include a wireless communications module.

device control logic includes operation logic, sensor logic, alarm logic, alert logic, and optionally, alert response logic, among other functions. device operation logic enables the device to function, managing power and communications between device components. The device sensor logic enables the operations logic to receive and analyze measurements from sensors in communication with the device. device alarm logic engages device audible, visual, tactile alarms and combinations thereof. The device alert logic engages device communications module to send an alert to a software application located on a remote device or remote monitoring system.

device control logic contains firmware for evaluating emergent medical conditions based upon multiple sensor inputs and is designed to integrate multiple signals such as oximetry data (i.e., pulse rate, oxygenation, respiration rate), as well as physical orientation data (i.e., standing, laying), mobility data (i.e., moving or unmoving), response data (i.e., lack of response to alarms or stimulation, lack of cancellation of alarm) or physical event data (i.e., potential fall) to determine whether patient is experiencing an emergent medical event. This “multi-threading” of multiple continuous streams of sensor data sets this device apart from traditional devices that utilize a single sensor input for their activity, such as ECG monitors.

device control logic may monitor multiple input signals utilizing both value thresholds and value trends. Additionally, control logic may utilize multiple input signals to confirm potential medical events prior to engaging alarm logic or alert logic. In some situations, the medical event is confirmed using multiple readings (i.e., pulse rate, respiration rate, oxygenation) from oximeters. In other situations, the medical event is confirmed using a lack of response to alarms or other stimulation (i.e., no-motion state, lack of alarm cancellation). In other situations, the medical event is confirmed by a combination of information from oximetry sensors and non-oximetry sensors or user response.

The communications module contains at least one form of wireless communication mechanism, such as Bluetooth radio, WLAN modem or cellular modem; and may include an antenna that is located peripherally to the radio/modem. The device communications module may enable engagement with other wireless communication devices (such as a mobile phone, home device, home network portal) or other medical devices during or after the medical event.

The device's physical form is optimized for patient comfort, size and privacy, with a streamlined and anatomically shaped design that minimizes the device's physical profile to enable patients to wear the device discretely (i.e., concealed under clothing).

The device may include one or more anatomically shaped platforms housing the device components, a wearable garment to contain the device platforms, and one or more mechanisms for securing the wearable garment to the patient.

Additionally, the device platform may contain a defined configuration of components to enable bilateral (left and right) use.

The wearable garment may include a mechanism for securing the device platform to the garment. The device platform and wearable garment is secured to the patient using cables, straps, velcro or other self-adhesive material, snaps, buttons, compression garments, constrictive bands, elastomeric materials, bi-stable springs and/or a combination of the preceding.

The device platform may include one or more anatomically shaped segments joined by a flexible fabric or other elastomeric material, or via inelastic joints such as hinges. The platform segments is oriented to one another along an arc measuring 45-180 degrees.

The dorsal (outer) side of the wearable platform may include a flexible covering (such as a fabric or silicone) or rigid covering (such as plastic or metal). The ventral (inner) side of the wearable platform includes ports for the sensor probes to access the patient's skin.

The device may be in communication with a cloud-based remote monitoring system to store, analyze and respond to sensor data. The remote monitoring system may possess additional user information, such as the user's medical condition or history, or may remove any identifiable data from alert response logic.

During a period of biometric readings within normal range (i.e., no medical event is indicated), the device communicates with the remote software application to upload sensor and device status data in regular, periodic intervals (i.e., once per hour) using the wireless communication module. If an emergency medical event occurs, the device alert logic sends a signal to the remote monitoring system using the device communications module (and associated device, if necessary), activating the remote software application alert response logic.

The system may include a mobile (smartphone) application and firmware for communicating with user's phone. The mobile application may display device status and user data after being retrieved from the device or cloud-based remote monitoring system. The mobile application may also enable the patient to directly contact the remote monitoring system (via the application user interface) or via their phone (i.e., voice call, text) for the purposes of receiving information or additional medical services. The mobile application may allow users to record medical symptoms and provide subjective descriptions of user experience using voice, text and photos. Additionally, the mobile application may allow users to record time events for known behaviors which may induce medical events (i.e., pharmacologic ingestion, physical exertion, emotional distress).

The method of operating the device involves receiving information from at least one oximetry probe in communication with the device. The method detects a medical event in the diagnostic information through operation of control logic in a wearable device. The method communicates an activation signal to the alarm module (comprising audible and haptic alarms) and wireless communication module (comprising of detected medical event information and wireless communication activation information) to activate alert response logic of a remote software application, located on a remote device or cloud-based remote monitoring system. The method requests additional interventions for the medical event through operation of the cloud-based system's alert response logic to contact designated emergency contacts, healthcare providers or emergency medical services.

The method of operating an Emergency Medical Monitoring System involves receiving information from the wearable device in communication with the remote software application, located on a remote device or remote monitoring system. The method prompts additional interactions with the patient in the first 24 hours following a medical event, through operation of the remote software application's alert follow-up logic that attempt to contact the user and/or user designated contacts. This capability may enable communication with a live operator (i.e., a person serving as moderator, navigator, or counsellor for the user) and may facilitate user acceptance of additional medical or psychological treatment, including “wraparound” social services, after a medical event.

The method of operating device mobile application involves activating the mobile application in response to an alert signal from the device, allowing the mobile application to contact the patient-designated contacts directly after the emergency medical event via the application user-interface.

The method of operating the device mobile application involves activating the mobile application in response to an alert signal from the device, allowing the remote monitoring system to contact the patient or patient-designated contacts directly after the emergency medical event via the application user-interface. This capability may improve efficiency for purposes of follow-up.

The system is combined with a wearable drug delivery system (DDS) that is designed to deliver medication for emergency medical events, into a desired tissue compartment or a specified body location. The device and DDS is collocated on the wearable device platform or collocated on the same body region using a compatible wearable garment or is located on a separate body location. The device may convey raw or processed biometric data, information and/or operation instructions from the device control logic to the control logic in a wearable drug delivery device.

The device is configured to send an activation signal to a remote operating system of DDS located on a different part of the user's body (i.e., device worn on upper arm and DDS worn on upper leg). The activation signal is transmitted to the DDS from the device using wired or wireless communication. Additionally, device is configured to send signal regarding DDS activation status (i.e., DDS standby, DDS engaged, DDS activated, DDS dose delivery imminent, DDS dose delivery in-process, DDS dose delivery complete) to a remote software application or remote monitoring system.

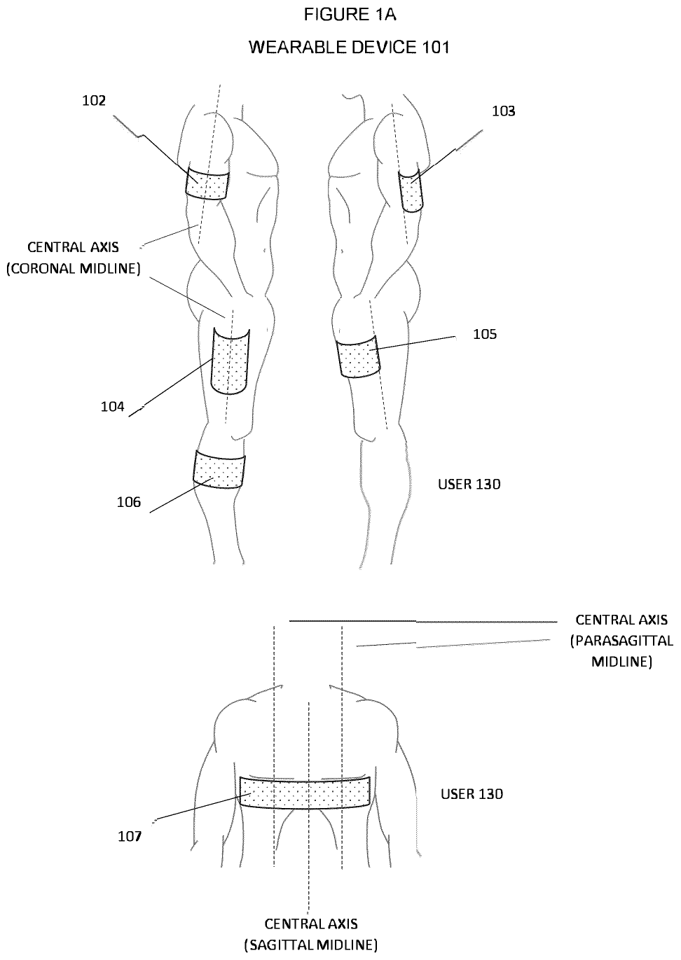

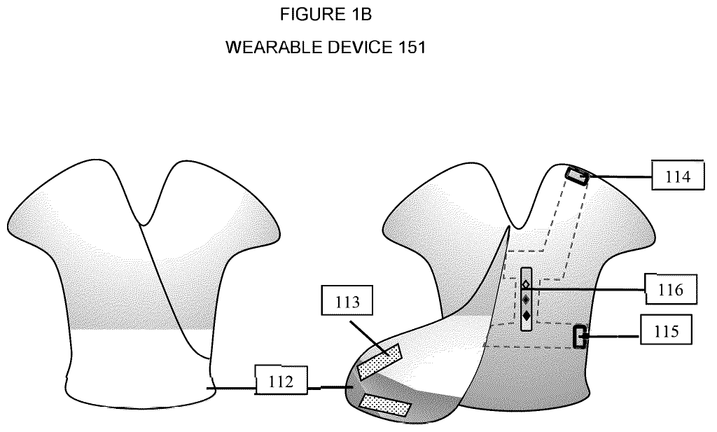

In FIG. 1A, the wearable device 101 is configured with specific contours to mount comfortably to a user's body. For example, a side view of User 130 illustrates Position 102 (lateral upper arm), Position 103 (posterior upper arm), Position 104 (lateral upper leg/hip), Position 105 (anterior upper leg), and Position 106 (lower leg) as possible locations for securely mounting the wearable oximetry device 101. An anterior view of User 130 illustrates Position 107 (anterior upper torso) along midsagittal plane (sternum and/or xyphoid Process) or along parasagittal plane (external oblique and serratus anterior) as possible locations for securely mounting the wearable device 101.

The Position 102 shows the wearable device 101 on User 130's lateral upper arm. The device is located along a central axis of the midline of the lateral upper arm (Coronal midline).

The Position 103 shows the wearable device 101 on User 130's posterior upper arm. The device is located along a central axis of the midline of the posterior upper arm (triceps).

The Position 104 shows the wearable device 101 located on the lateral upper leg or hip (vastus lateralis) of the User 130. The device 101 is located along a central axis of the midline of the lateral thigh (Coronal midline).

The Position 105 shows the wearable device 101 on the user 130's anterior thigh. The device 101 is located along the midline of the anterior thigh.

The Position 106 shows the wearable device 101 on the user 130's lower leg. The device 101 is located along the midline of the lower leg.

The Position 107 shows wearable device 101 on User 130's anterior upper torso. The device 101 is located along midsagittal plane (sternum and/or xyphoid process) or along parasagittal plane (external oblique and serratus anterior).

In other configurations, the wearable device 101 is used in other body locations, such as the lower back, upper back (trapezius), ventrodorsal gluteal (buttocks), abdomen or forearm.

The wearable device 101 may have identifying marks (i.e., fabric, reflective tags, lights, indentations, or other marks) on the exterior of the device for the purpose of identifying device location on the body and relative position. Such marks are utilized to assist the wearer in correct positioning of the device.

The Emergency Medical Monitoring System 100 may be used with a mobile application to determine body location and relative position of device using the User's smartphone camera. Such a mobile application may use identifying marks on the device to determine body location and relative position.

wearable device 101 may contain one or more sensors such as accelerometers, gyroscopes, magnetometers (AGM) or a combination thereof, to allow device control logic to determine relative location of oximetry probes to target tissues and User body location, enabling the device to be repositioned across different body locations. For example, a device worn on the upper arm may use AGM measurements to allow device Control Logic to determine whether the device is positioned on the left or right arm based upon device initialization,

FIG. 1B illustrates a wearable device 151 in accordance with one embodiment. The external layer 112, visible to user, is designed to conceal any obvious medical device technology. External layer 112 is made of soft, washable, breathable material that is appealing to touch, for overnight wear. It is a combination of materials including cotton, elastane (spandex), polyester, or other synthetic material.

The wearable device 151 may come in several sizes (extra small, small, medium, large, extra-large) to accommodate the range of adolescent, young adult and adult populations that are most likely to be at risk for emergency respiratory decline such as opioid overdose or post-ictal complications.

FIG. 1B illustrates one embodiment of wearable device 151 in which the external layer 102 is two parts; the front part may open at attachments 103 to reveal a back part that shows an easily accessed console with user interface, including device status and alarm lights 116, appropriate for an unobtrusive device (e.g., battery power can be viewed by User and then the light covered). The lights 116 will notify of device status without disrupting night vision and will be clearly identifiable when viewed by User (after removing the front part of external layer 102). The wearable device 110 may have identifying marks (i.e., varied fabric, reflective tags, lights, indentations, or other marks) on the Exterior Layer of the wearable Proposed device for the purpose of identifying component (e.g., sensors, alarm button) locations on the body and their relative position and status. Such marks are utilized to assist the wearer in correct positioning of device 151.

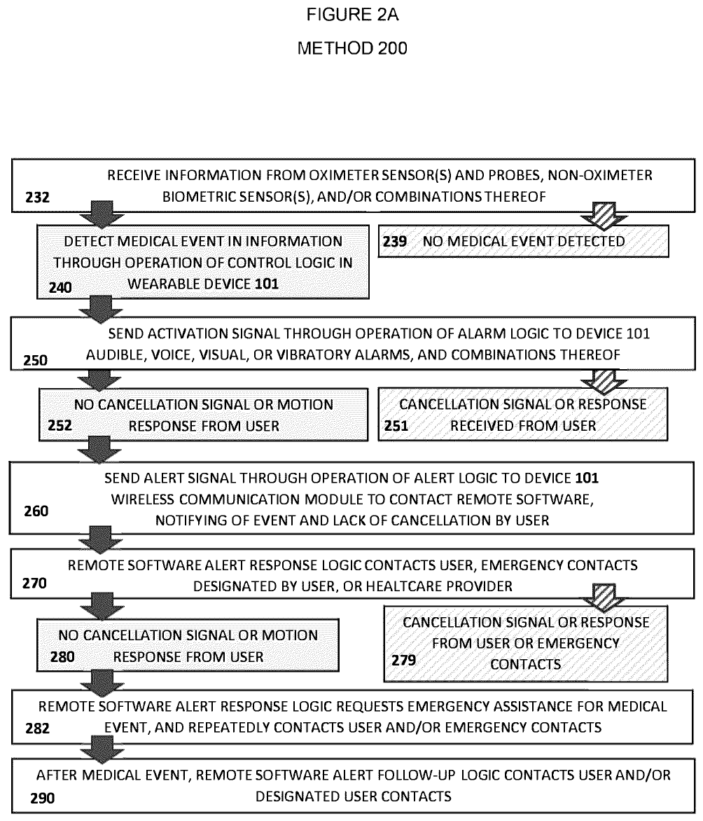

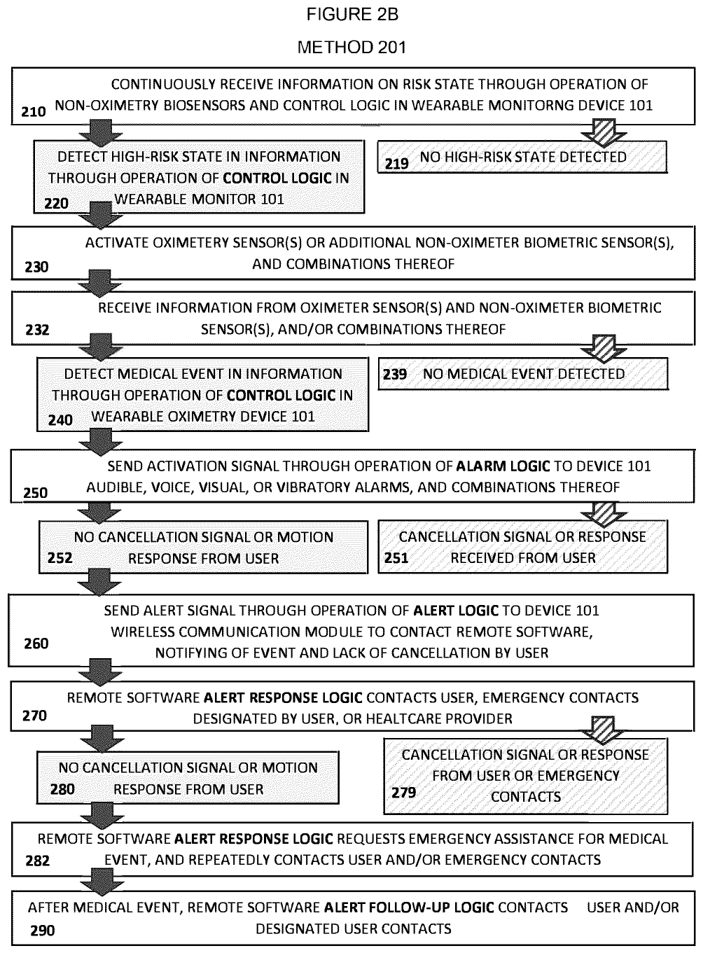

In FIG. 2A, the wearable device is operated in accordance with Method 200, the process disclosed in FIG. 2. In Block 232, a Method 200 for operating a wearable device involves receiving information from at least one oximetry probes, at least one non-oximetry biometric sensor or external environment sensor, and/or combinations thereof.

In Block 240, the Method 200 detects a medical event in the information through operation of control logic in a wearable device 101.

In Block 250, the Method 200 communicates an alarm activation signal to the audible, visual and tactile alarms (vibratory alerts).

In Block 251, the Method 200 accepts a cancellation signal from the User and communicates the cancellation signal to the remote software application as part of device status information at next regular upload.

In some embodiments, the functions of the remote software application may be performed by Device Logic.

In Block 252, the Method 200 fails to receive a cancellation signal or response from the User and device continues in alarm state.

In Block 260, the Method 200 communicates alert activation signal from device 101 comprising detected medical event information (and lack of Alarm cancellation by User) to remote monitoring system.

In Block 270, the Method 200 repeatedly requests communication with wearer, emergency contacts, healthcare provider, or combinations thereof, via voice, or text, or mobile application using the alert response logic of the remote software application or remote monitoring system, or in some configurations, using the device wireless communications module.

In Block 279, the Method 200 accepts a cancellation signal from the User and communicates the cancellation signal to the remote software application.

In Block 280, the Method 200 fails to receive a cancellation signal or response from the User and device continues in alarm (and alert) state.

In Block 282, the Method 200 requests additional interventions such as emergency live assistance (i.e., 9-1-1) for the medical event using the Alert Response Logic of the Remote Monitoring System.

In Block 290, the Method 200 requests communication with user, or emergency contacts designated by user, within 24 hours of the medical event, (via voice, or text, or mobile application) using the Alert Follow-Up Logic of the Remote Monitoring System. In some configurations, User contact can be made using the wearable oximetry system wireless communications module.

In FIG. 2B, the wearable device is operated in accordance with Method 201, the process disclosed in FIG. 2B.

In Block 210, a Method 201 for operating a wearable device involves receiving continuous information from at least one non-oximetry biometric sensor, or external environment sensor, and/or combinations thereof.

In Block 219, a Method 201 does not detect a high-risk state.

In Block 220, a Method 201 detects a high-risk state after receiving information from at least one non-oximetry biometric sensor, or external environment sensor, and/or combinations thereof.

In Block 230, a Method 201 detects a high-risk state after receiving information from at least one non-oximetry biometric sensor, or external environment sensor, and/or combinations thereof.

In Block 232, a Method 201 activates oximetry sensor(s), additional non-biometric sensors, or a combination thereof.

In Block 239, a Method 201 does not detect a high-risk oxygenation or respiration state.

In Block 240, the Method 201 detects a medical event in the information through operation of control logic in a wearable device 101.

In Block 250, the Method 201 communicates an alarm activation signal through operation of alarm logic to the audible, visual and tactile alarms (vibratory alerts).

Following activation signal, the Method 201 proceeds as per the Method 200.

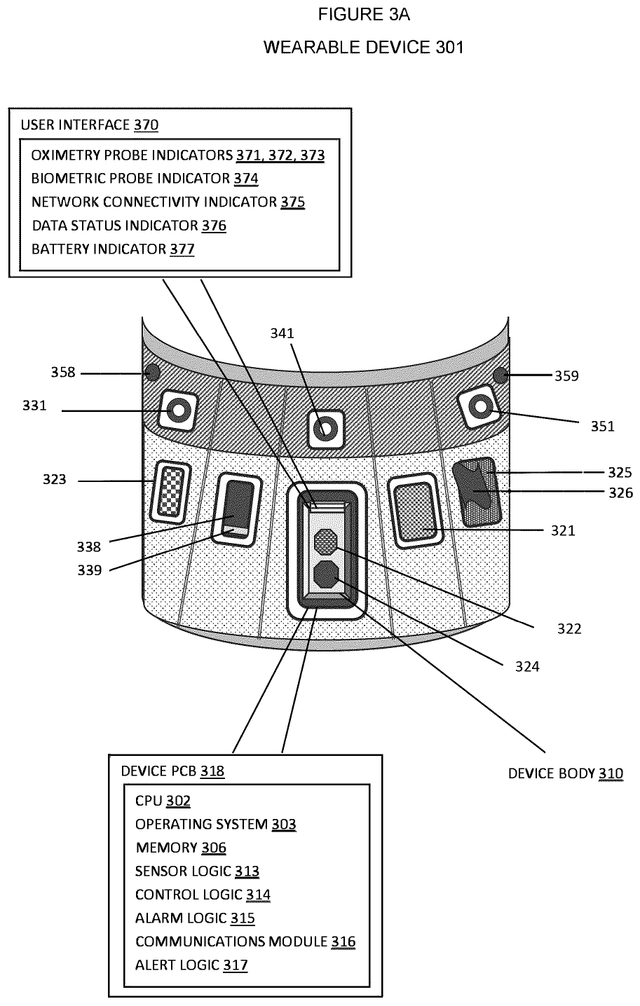

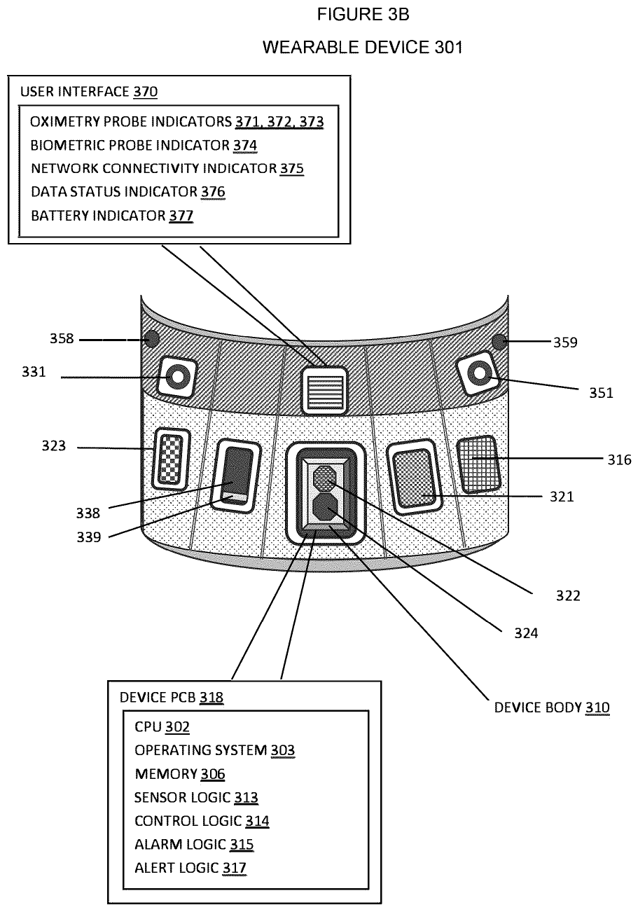

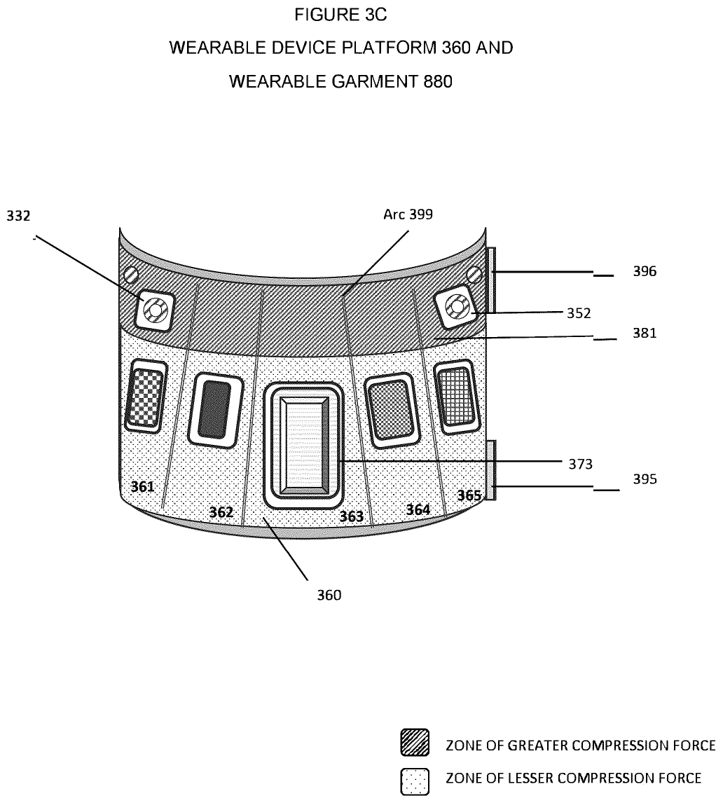

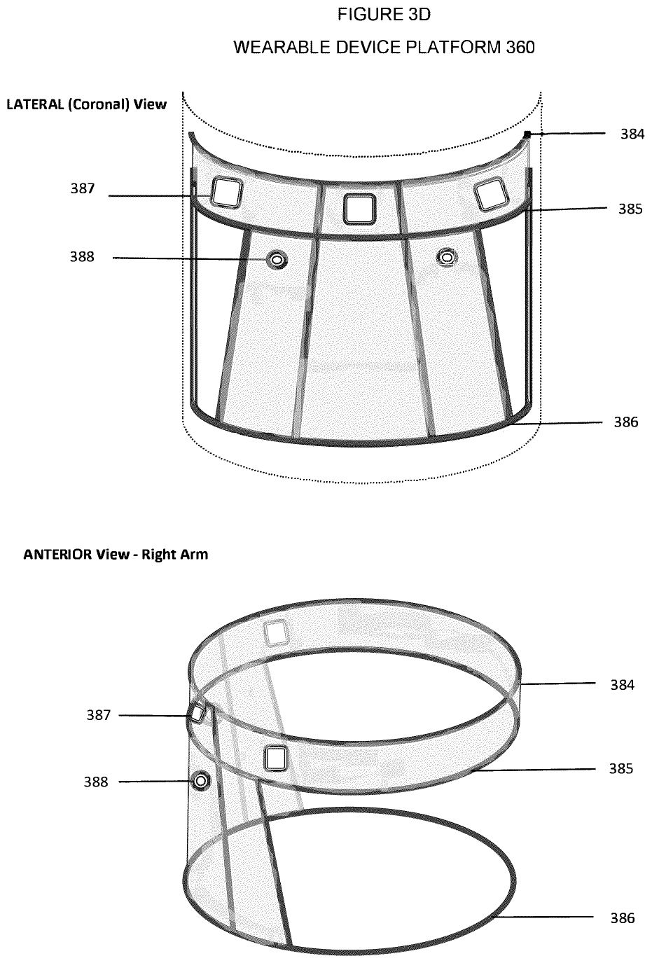

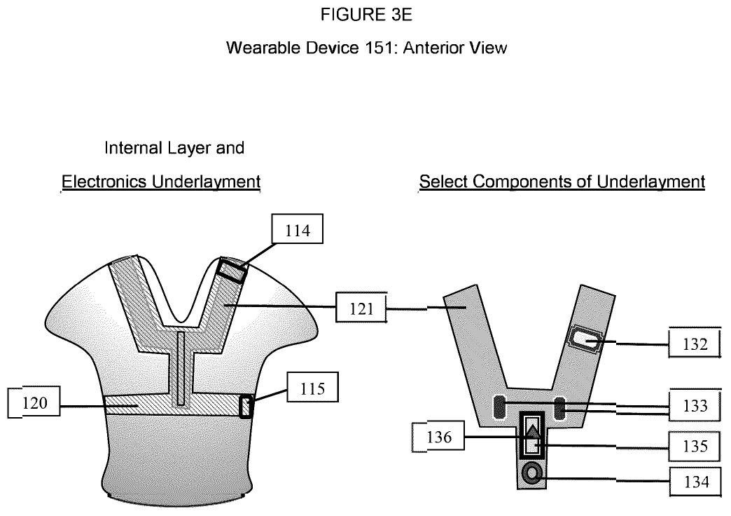

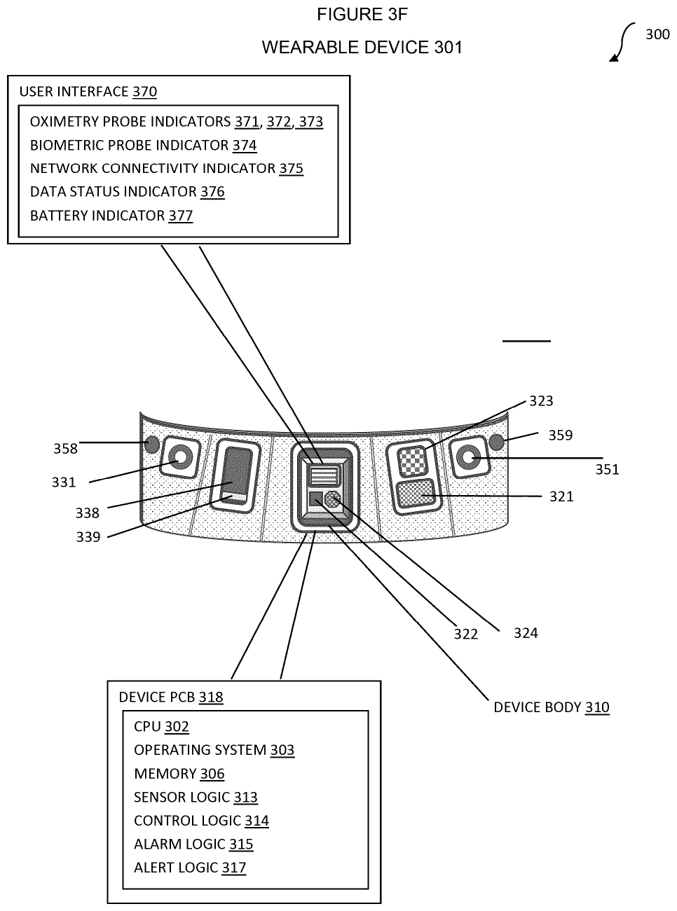

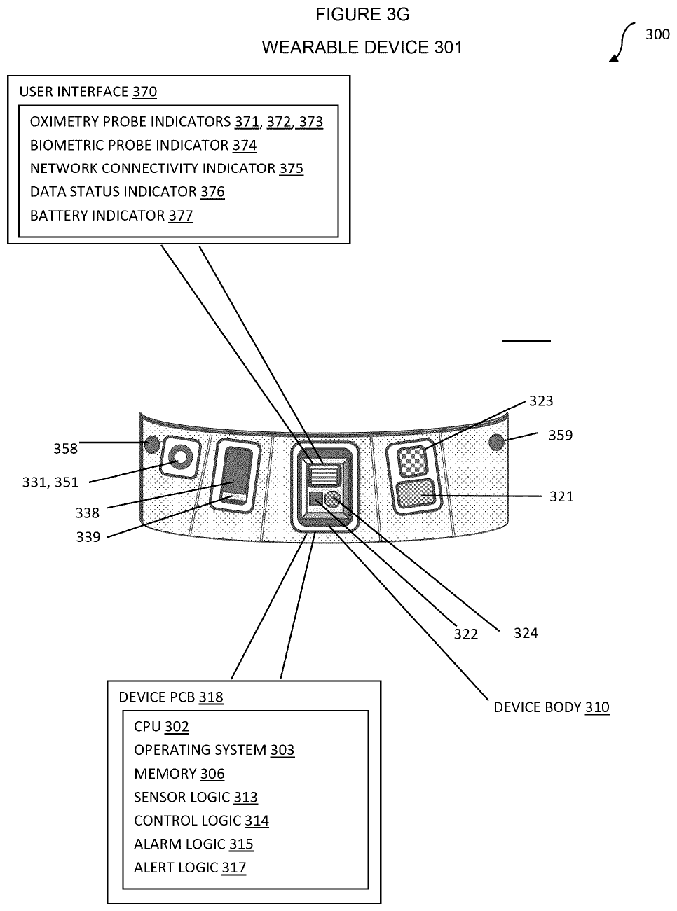

FIG. 3A illustrates one embodiment of the wearable device 301, comprising at least two oximetry probes (331, 341 and 351) and at least two non-oximetry biometric sensors 358 and 359. The device body 310 contains a device PCB 318, alarms 321 and 323, and a user-interface 370.

The device PCB 31g contains the device CPU 302, operating system 303, memory 306, sensor logic 313, control logic 314, communications module 316, alarm logic 315 and alert logic 317.

The at least two oximetry probes 331, 341 and 351 are communicably coupled to device PCB 318. The at least two oximetry modules 330, 340 and 350 are PPG. NIRS or a combination of the two. For example, device 301 is communicably coupled to two PPG oximetry Modules and one NIRS module. In another example, device 301 will be communicably coupled to two NIRS Modules and one PPG module. In another example, device 301 will be communicably coupled to three PPG oximetry Modules. In another example, the device 301 will be communicably coupled to two NIRS oximetry Modules.

The at least two oximetry Modules 330, 340 and 350 are communicably coupled to one or more oximetry Probes 331, 341 and 351 and device PCB 318. The device 301 utilizes reflective PPG sensors (i.e., PPG sensors configured to emit and detect light on a single sensor face) rather than transmittance PPG sensors (i.e., PPG sensors configured to emit light on one side of the tissue, such as a fingertip bed, and detect it on the other side).

In some embodiments, the at least two oximetry probes may be co-located within a single housing. In one example, the at least two oximeter probes are comprised of one probe with one set of photo emitters and photo receivers oriented in an approximately 180-degree orientation from a second probe with a second set of photo emitters and receivers in the same spectrum. In another example, the at least two oximeter probes are comprised of one probe with one set of photo emitters and photo receivers oriented in an approximately 90-degree orientation from a second probe with a second set of photo emitters and receivers in the same spectrum.

In some configurations, the device 301 may utilize NIRS and PPG sensors configured with detectors and emitters designed for use in adult or pediatric patients, or a combination thereof. For example, device 301 may utilize reflective PPG sensors designed for use in adult patients and NIRS sensors designed for use in pediatric patients.

The oximetry probes 331, 341 and 351 are configured for locations on wearable device 301 to minimize electromagnetic noise and any additional artifacts from movement and light scatter. In some configurations, the oximetry probes 331, 341 and 351 are spaced at least 60 mm apart. In some configurations, the anatomical shape of device platform 360 may enable oximetry probes 331, 341 and 351 to be located less than 60 mm apart. For example, when worn on the upper arm, oximetry probes are less than 60 mm apart, because they are located on opposite sides of the Coronal planes (i.e., oximetry probe 331, located on lateral bicep less than 60 mm from oximetry probe 351 located on lateral triceps).

The oximetry probes 331, 341 and 351 are configured for sampling intervals on wearable device 301 to minimize electromagnetic noise and any additional artifacts from movement and light scatter. In some configurations, the body location of wearable device 301 may allow for oximetry probes may allow for probes to be located less than 60 mm apart. For example, when positioned on the upper arm, oximetry probes 331, 341 and 351 are less than 60 mm apart from one another because of the location of each oximetry probe on a distinct target tissue (i.e., posterior triceps, lateral triceps, lateral biceps).

In some configurations, the asynchronous use of oximetry probes may allow for probes to be located less than 60 mm apart. In some configurations, the oximetry probes 331, 341 and 351 are configured to sample asynchronously. For example, when configured to sample asynchronously, oximetry probes 331 and 351 are located less than 60 mm apart from probe 341, because probes 331 and 351 may sample simultaneously, followed probe 341 sampling asynchronously from the other probes.

The oximetry Probes 331, 341 and 351 may comprise at least one near-infrared spectroscopy (NIRS) probe, wherein the probe contains at least two photo emitters and two photo detectors. In some configurations, the probes 331, 341 and/or 351 are configured with at least one NIRS photo emitter located more than 20 mm from one NIRS photo detector and more than 30 mm from another NRS photo detector. For example, when at least one oximetry Probe 341 is a NIRS probe, the photo emitters of oximetry Probe 341 is located 25 mm and 40 mm away from the two NIRS photo detectors.

In some configurations, the probes 331, 341 and/or 351 is configured with at least one photo emitter located 10-20 mm from one photo detector and 20-30 mm from another photo detector. For example, when at least one oximetry probe 341 is a NIRS probe, the photo emitter of oximetry probe 341 is located 15 mm and 25 mm away from the two photo detectors.

The at least one non-oximetry biometric sensor 312 is communicably coupled to one or more biometric probes 358 and 359 and device PCB 318. In some configurations, the biometric sensor 312 is an accelerometer, gyroscope and/or magnetometer (AGM). In some configurations, the biometric sensor is a temperature state sensor, and the biometric probes is a thermometer. In some configurations the biometric sensor is an electrodermal activity monitor. In some configurations the biometric sensor may comprise multiple sensors. For example, biometric sensor 312 may comprise an accelerometer, gyroscope and/or magnetometer, and a thermometer probe 358 and 359 as thermometer probes. In some configurations, biometric sensor 312 is communicably coupled to more than two biometric probes. For example, biometric sensor 312 may comprise a thermometer located on device PCB 318, an accelerometer, gyroscope and/or magnetometer connected to biometric probes 358 and 359; and an electrodermal activity monitor located on device Body 310 connected to additional Biometric Probes (not shown) from at least two one oximetry Sensors.

The control logic 314 enables the wearable device 301 to receive information from at least two oximetry Sensors and at least one non-oximetry biometric sensor using sensor logic 313, and subsequently analyze the vital sign data for thresholds or trendlines that are outside of the acceptable range. In the event that vital sign data is nominal, the control logic stores the data in memory 306 until it is transmitted to a remote software application, remote monitoring system and/or a remote storage device. In the event the vital sign data is outside the acceptable range, the control logic 314 activates alarm logic 315 and/or alert logic 317.

The control logic 314 is configured to operate oximeter probes in a specified sampling regime (i.e., sequential, or staggered intervals) to prevent interference from one another, or the communications module 316. In some configurations, the oximeter modules and probes is configured to use a method of sampling such that the oximetry data is captured and analyzed by device logic in approximately 10-12 second intervals, with active data capture occurring for approximately 8-10 seconds followed by 2 seconds without sampling to allow the target tissue to allow any remaining light artifact from the probe or target tissue to dissipate. For example, in a three-probe configuration, oximetry probe 331 captures time 0:01-0:08 of emergency medical event; pause 0:09-0:10; oximetry probe 341 captures 0:11-0:18; pause 0:19-0:20; oximetry probe 351 captures 0:21-0:28; pause 0:29-0:30; then the cycle repeats: probe 331 captures 0:31-0:38, probe 341 captures 0:41-0:48, probe 351 captures 0:51-0:58).

In some configurations, following detection of a high-risk state, the control logic 314 is configured to operate oximetry modules and oximetry probes at a less frequent interval during a period of time in which vital sign data is within acceptable range, and operate oximetry modules and oximetry probes at a more frequent interval after a vital sign threshold or trendline is measured outside of the acceptable parameters. For example, the control logic 314 is configured to operate oximetry modules 330 and 340, and oximetry probes 331 and 341, to measure every 180-240 seconds after a period in which vital sign data has remained within acceptable parameters for 2-3 hours. Upon detecting a vital sign threshold or trendline outside of acceptable parameters, the control logic will operate oximetry module 330 and 340, and oximetry probe 331 and 341 to measure every 30-60 seconds.

In some configurations, the control logic 314 is configured to operate a subset of oximetry modules and oximetry probes under nominal conditions, only operating the remaining oximetry modules and oximetry probes after a vital sign threshold or trendline is measured outside of the acceptable parameters. For example, the control logic 314 is configured to operate oximetry modules 330 and 340, and probes 331 and 341, upon detection of high-risk state from non-oximetry biosensors, and upon detecting a vital sign threshold or trendline outside of acceptable parameters, the control logic will also operate oximetry Module 350 and oximetry probe 351 to help monitor medical event.

In some configurations, sensor logic 313 is configured to utilize a subset of oximetry modules and oximetry probes to confirm the vital sign threshold or trendline measurement of another subset of oximetry modules and probes, prior to vital sign threshold or trendline measurements being determined as within or outside acceptable parameters. For example, the control logic 314 is configured to operate oximetry modules 330 and 340, and probes 331 and 341, under nominal conditions, and upon detecting a vital sign threshold or trendline outside of acceptable parameters, the control logic will operate oximetry module 350 and oximetry probe 351 to confirm the measurement of oximetry probes 331 and 341, prior to engaging alarm logic 315 or alert logic 317.

In some configurations, sensor logic 313 is configured to reflect specific target tissues of interest for acquiring oxygenation measurements. oximetry data reflects oxygenation of underlying target tissue based upon a calibration curve for the optical sensors that is specific to a given target tissue. In some configurations, sensor logic 313 may include additional algorithms, static or dynamic measurement adjustments, or additional calibration curves to reflect the specific oximeter probe locations or target tissues, as determined by the control logic 314. For example, if the control logic 314 is configured for device 301 placement on the upper arm, device firmware will derive the relative orientations of oximetry probes 331, 341 and 351 to determine the associated target tissue for each probe (i.e., for right upper arm, probe 331 targets lateral triceps tissue, probe 351 targets the brachialis, lower deltoid and/or surrounding tissue, and probe 341 targets lateral biceps tissue). Control logic 314 will apply a target tissue specific algorithm to the measurement of oximetry variables, and vital sign thresholds and trendlines used in device logic, to determine if the measurement is within acceptable parameters.

In some configurations, sensor logic 313 may include additional algorithms (i.e., static or dynamic measurement adjustments, or additional calibration curves) reflecting specific ranges of melanin in target tissues (i.e., specific ranges of skin tones, such as used in the Fitzpatrick Scale medical model). PPG and NIRS oximetry measurements is affected by the amount of melanin present underneath each oximetry Probe. In some configurations, control logic 314 and/or sensor logic 313 may include algorithms for determining melanin concentration, skin tone, pigmentation based upon measurements from one or more oximetry probes, measurements based upon at least one non-oximetry biometric sensor (i.e., a CMOS image captured by an optical camera), and/or based upon information received from remote software application, remote monitoring system or mobile application. For example, if the sensor logic 313 is configured for wearable device 301 in a User 130 with Fitzpatrick Scale V skin tone, device firmware will apply an additional algorithm to gather data from oximetry probes 331, 341 and 351, and apply an algorithm specific to the Fitzpatrick Scale V skin tone to the vital sign thresholds and trendlines used in device logic, to best determine if the measurement is within acceptable parameters.

The communications module 316 enables the wearable device 301 to communicate with a remote software application or remote monitoring system by way of a wireless network. The communications module 316 may contain one or more wireless communication methods (i.e., WLAN/BLE modems and cellular modems), and may also enable communication with a wearable oximetry Mobile Application located on a user's mobile phone and user's other Connected devices.

device 301 may contain alarm system 320, which may include a series of alarm mechanisms to engage the user, bystanders or medical providers to identify and respond to a medical event. The alarm system 320 may contain audible alarm 321, visual alarm 322, tactile/haptic alarm (vibratory alert) 323, and an alarm cancel/reset Button 324.

Audible alarm 321 is configured to produce a series of tones in response to medical events. In some configurations, the volume of the audible alarm 321 may escalate, and the frequency of the audible alarm 321 may oscillate, or a combination of thereof. The audible alarm of 321 is configured for purpose of drawing attention of bystanders or medical responders that User 130 in need of medical assistance. The audible alarm of 321 is configured for the purpose of gaining the attention of User 130 to prompt a response and determine if they need medical assistance

The series of tones produced by audible alarm 321 is configured to correspond to ISI/IEC 60601-1-8 standards for alarms in medical equipment, including general medium urgency (3 short tones), general high urgency (3 short fast tones, 2 second pause, 2 short fast tones), oxygenation medium alarm (3 short descending tones), oxygenation high alarm (3 short fast descending tones, pause, 2 short fast further descending tones), power failure medium alarm (1 high tone followed by 2 low tones), and power failure high alarm (1 high tone followed by 2 low tones, 2 second pause, 1 high tone followed by 1 low tone), among others.

Audible alarm 321 is also configured to produce a recorded voice message or simulated voice to verbally identify emergency medical event, provide information regarding event, request cardio-pulmonary resuscitation (CPR) or rescue breathing, provide information or instruction regarding CPR or rescue breathing, request help or contact of emergency medical assistance (i.e., 9-1-1), request or suggest rescue medicine administration, and provide instructions on administration of rescue medication.

Visual alarm 322 is configured to produce a series of LED lights in response to medical events. In some configurations, the brightness/intensity of the LED lights may oscillate (i.e., flashing, pulsating) for the purpose of gaining attention of User 130 to determine if they need medical assistance, or drawing attention of bystanders or medical responders that User 130 is in need of medical assistance.

Tactile/haptic alarm (vibratory alert) 323 is configured to produce a vibratory stimulation to User 130 in response to medical events. In some configurations, the strength/intensity of the vibratory mechanism may oscillate for the purpose of gaining attention of User 130 to determine if they need medical assistance.

User interface 370 may include LED lights or visual displays (i.e., LCD, LED, OLED, or other display method), by which the ready status of the device is ascertained, such as oximeter probe status 371, 372 and 373, non-oximetry biosensor status 374, network connectivity status 375, data upload status 376 and battery status 377.

In some configurations, device body 310 may include openings or transparent windows, through which indicator lights or displays is viewed. While the physical appearance of indicator lights and windows are described, it is contemplated that alternate locations, colors, shapes or presence/absence of indicator lights and windows is provided.

User interface 370 is also located on a remote software application on a remote device communicably coupled to wearable device 301.

User interface 370 may include configuration of information display to avoid confusion by the user (i.e., the use of green and yellow indicator lights for status, reserving red indicator lights for alarms and alerts; the use of shaped lights to indicate specific oximeter probe status; and the use of shaped lights or symbols to indicate status of specific device components such as battery and network connectivity).

In some configurations, the indicator lights for oximeter probe status 371, 372 and 373, and non-oximetry biosensor status 374 is designated as green (ready/active) and yellow (not ready/malfunctioning/not active). In some configurations the physical shape of indicator lights for oximeter probe status 371, 372 and 373 may also indicate the location of the probe. For example, for a device 301 worn on the right upper arm, the oximetry probe status indicator 371 (corresponding to oximetry probe 341, located posterior) is shaped as a triangle, oriented to point towards the posterior of the device; the probe status indicator 372 (corresponding to oximetry probe 342, located laterally) is shaped as a triangle, oriented to point outward, toward the middle (lateral) section of the device; and the probe status indicator 373 (corresponding to oximetry probe 351, located anterior) is shaped as a triangle, oriented to point towards the anterior of the device.

In some configurations the indicator light for device battery and charge status 377 is designated as a series of colored bars, displaying approximate charge level of the device. For example, the indicator light for device battery 377 is a series of five colored bars, each representing 20% power remaining: the 60-80% and 80-100% power remaining bars colored green; the 20-40% and 40-60% power remaining bars colored yellow; and the 0-20% power remaining bar colored flashing yellow.

In some configurations, the indicator lights for communications module network connectivity status 375, and data upload status 376 is designated as green (ready/active) and yellow (not ready/malfunctioning/not active). For example, the indicator light 375 is configured to display a solid green light when connected to a mobile network, and to display a yellow light when not connected; and indicator light 376 is configured to display a green light when the most recently attempted data communication with remote software application or remote operating system was successful, to display a yellow light if the most recent recently attempted data communication with remote software application or remote monitoring system was unsuccessful; and to display flashing yellow if data upload has been unsuccessful for more than four consecutive hours.

The user Interface 370 is oriented to allow the wearer to easily view the indicator lights or display. In some configurations, User interface 370 is located on the proximal and superior side of the device. For example, when wearable device 301 is located on the upper limb, User Interface 370 is located on the proximal (upper) side of the device, most visible from the user's point-of-view in a standing position.

In other configurations, user interface 370 is located on the medial or lateral side of the device. For example, when the wearable device 301 is located on the lateral left upper leg, user interface 370 is located on the medial side of the device, and when the wearable device 301 is located on the lateral right upper leg, the User interface 370 is located on the lateral side of the device, still easily visible from the point-of-view of the user when in a sitting position.

In some configurations, when wearable device 301 is worn on the upper torso, user interface 370 is located on the superior side of the device, most visible from the top-down point-of-view of the user when in a standing position. In other configurations, user interface 370 is located on a peripheral display unit, which is attached to the device 301 via wired or wireless communication, allowing the user interface 370 to be visible from a greater number of viewing angles.

In some configurations, wearable device 301 may provide a large print surface for labelling. Labelling may include device instructions 325. The instructions is visible or concealed beneath a peel-away or other opaque instructions cover 326 which may also contain device manufacturer Logo/Trademark.

In some configurations, the instructions labelling cover 326 may contain a mechanism to enabling the device instructions 325 to be advanced and retracted. For example, the instructions labeling cover 326 may contain a spring mechanism to allow User 130 or bystanders to display device instructions 325 by pulling on one end of the instructions 325, and to conceal device instructions 325 by engaging the spring mechanism to retract device instructions 325 into device instructions cover 326. In another example, the instructions labeling cover 326 may contain a mechanism to allow User 130 or bystanders to display device instructions 325 by manually moving (advancing) a wheel, dial or other means of pushing instructions 325 out of cover 326, and to conceal device instructions 325 by moving (revering) wheel, dial or other means to retract device instructions 325 into device instructions cover.

The device 301 contains a charging port 337. The charging port is configured for any standard charging mechanism, such as a USB cable plug 2.0 or 3.0. One aspect of the wearable device 301 is that the power supply may include one or more batteries or energy storage devices. The batteries or energy storage devices is rechargeable or a mixture of rechargeable and disposable batteries. The batteries or energy storage devices is recharged via a connection to an external power source (such as a USB connection to AC power). In some configurations, the wearable device 301 includes independent power supplies for one or more components. For example, a wearable device 301 is configured to have Alarm system 320 possess an independent power supply. In this situation, the Control Logic optimizes the use of non-oximetry sensors and oximetry sensors to balance battery life and sampling frequency without compromising the Alarm functions of the device.

In some configurations, wearable device 301 is powered by a single, integrated power supply and charging mechanism 338. The power supply is co-located within the device Body 310, or on a separate wearable device platform segment.

In some configurations, wearable device 301 is powered by multiple independent power supplies. Power supplies is co-located or is located on separate wearable platform segments. Power supplies is shared by specific device components. For example, the CPU 302, operating system 303, non-oximetry sensors 312 and non-oximetry probes 358 and 359, and communications module 316 may share one power supply, while the oximeter modules 330, 340 and 350 and oximeter probes 331, 341 and 351 shares another power supply.

In another example, the oximeter modules 330 and 340, and the oximeter probes 331 and 341 may share one power supply, and the oximeter module 350 and oximeter probe 351 may share another power supply. In this situation, oximeter module 350 and oximeter probe 351 is configured to operate less frequently, thus decreasing their need for power, and oximeter modules 330 and 340, and oximeter probes 331 and 341 may operate more frequently, requiring more power.

In some configurations of the above example, the oximeter modules 330 and 340 are PPG oximeters, and the oximeter probes 331 and 341 are PPG oximeter probes, and the oximeter modules 350 are NIRS oximeters, and the oximeter probes 351 are NIRS oximeter probes.

In some configurations, wearable device 301 may include a backup power supply. The backup power supply is collocated with the main power supply 338 or located on a separate wearable device platform. The backup power supply is configured to automatically engage or is manually activated.

In some configurations, the backup power supply is configured to automatically engage upon discharge of the main power supply. In this situation, the device would alert the wearer to the main power supply status and engage the backup power supply to maintain device functionality and data upload. For example, the device may alert the patient regarding power supply status and engagement of the backup battery by various methods, including audible, visible and tactile alarms (vibratory alerts); and by notification via mobile application or text message.

In other configurations, the backup power supply is engaged manually. The patient or a bystander may engage the backup power supply by physically interacting with the device (i.e., removing a pull-tab separating a coin-style battery from the device electrodes). In this situation, the device is configured to automatically engage the control logic 314 and/or communications module 316 upon activation of the backup power supply.

In some configurations, the device PCB 318, body 310 and oximetry modules 330, 340 and 350 contain electromagnetic shielding. In some configurations, the communications module 316 is located in a separate housing external to the device Body 310, to minimize any potential electromagnetic interference.

FIG. 3B illustrates one embodiment of the wearable device 301, comprising at least two oximetry probes (331 and 351) and at least two non-oximetry biometric sensors 358 and 359.

The at least two oximetry probes 331 and 351 are communicably coupled to device PCB 318. The at least two oximetry modules 330 and 350 is PPG, NIRS or a combination of the two.

For example, device 301 is communicably coupled to one PPG oximetry Modules and one NIRS module. In another example, device 301 will be communicably coupled to two PPG modules. In another example, device 301 will be communicably coupled to two NIRS oximetry modules.

The at least two oximetry Modules 330 and 350 are communicably coupled to one or more oximetry Probes 331 and 351 and device PCB 318. The device 301 utilizes reflective PPG sensors (i.e., PPG sensors configured to emit and detect light on a single sensor face) rather than transmittance PPG sensors (i.e., PPG sensors configured to emit light on one side of the tissue, such as a fingertip bed, and detect it on the other side).

In some configurations, non-oximetry components of device 301 are distributed across multiple segments. For example, tactile/haptic alarm (vibratory alert) 323, power supply 338, device body 310 and device PCB 318, audio alarm 321, and wireless communication 316 are located on distinct platform segments.

FIG. 3C illustrates one embodiment of a wearable device platform 360 and a wearable garment 380 for device 301, device platform 360 comprises platform segments 361, 362, 363, 364 and 365; oximetry probe platforms 332, 342 and 352; and a device body platform 373. FIG. 3C also shows a wearable garment 380 for the purpose of securing device 301 to User 130, including wearable garment compression zones 381 and 383; garment closure mechanisms 395 and 396; and restraint mechanisms 391 and 392 (not shown), The ventral (interior) side of wearable garment 380 contains ports for oximetry and non-oximetry biometric sensor probes to access User 130's skin. The exterior of wearable garment 380 may also contain labeling, including instructions for use and manufacturer logo.

In some configurations, the wearable device platform segments 361, 362, 363, 364 and 365, is anatomically shaped to improve the comfort and minimize the physical profile of the device at the chosen body location.

In some configurations, the platform segments are aligned along an Arc 399 measuring the span of the wearable device platform 360, The device platform segments provide support to the component platforms while also providing comfort to User 130.

In some configurations, the wearable device platform 360 may include component platforms 332, 333, 342, 343, 352, and 353, among others, embedded within the platform segments to improve sensor-skin contact, protect components from damage, and distribute component weight while being worn at the chosen body location.

device body 310 is supported by device body component platform 373; oximetry modules 330, 340 and 350 are supported by oximetry module component platforms 333, 343 and 353 (not shown); and oximetry probes 331, 341 and 351 are supported by oximetry probe component platforms 332, 342 and 352.

In some configurations, the wearable device platform 360 is contained within a wearable garment 380. The wearable garment 380 enables the device platform 360 to support the device components and secure the device 301 to the user. The wearable garment 380 may also comprise a design that provides varying degrees of compression (i.e., the force experienced by the device components against the user's skin) in different body regions. For example, as shown in FIG. 3C, the wearable garment 380 comprises a zone of comparatively higher compression 381 near the proximal region and a zone of comparatively lower compression 383 near the distal region of the Garment.

In some configurations, the wearable garment 380 may also comprise a design that includes passive and active methods of restraint to secure device 301 to User 130. For example, the wearable garment 380 may employ an “active” restraint (i.e., restraints that respond to changes in tissue conformation, such as caused by movement, without any additional action being taken by User 130) such as a spring, bi-stable spring or elastomeric band, or a “passive” restraint (i.e., restraints that do not respond to changes in tissue conformation without any additional action being taken by User 130), such as adhesive, a strap or belt secured by a ratchet or buckle, a strap comprising an elastomeric material, a “clamp” or “clamshell” design, or combinations of the preceding active and passive systems.

In some configurations, the wearable garment 380 may form a continuous band around the limb of User 130, wearable garment 380 has at least one ventral (underside) port for each oximeter probe 331, 341 and 351, and may have additional ventral ports for the at least one non-oximeter biometric sensor probe 358 and/or 359.

FIG. 3D illustrates one configuration of wearable device platform 360 designed to provide structural support for wearable device 301 and/or individual device components using a semi-flexible frame, superstructure, and/or underlayment. This semi-flexible structural support comprises a proximal (superior) structural support 384, mid-segment structural support 385, distal (Inferior) structural support 386, oximetry probe housing interlock 387 and other non-oximeter biometric probe housing interlock 388.

In some configurations, wearable device platform 360 may comprise part of a multi-layer garment, wherein the platform 360 may comprise the outer (exterior) side of device 301; the oximetry probes and non-oximetry biometric probes are located ventral (interior) to the platform 360; and the device body 310 is located ventral (interior) to the platform 360.

In some configurations, wearable device platform 360 may comprise part of a multi-layer garment, wherein the platform 360 may comprise the inner (interior) side of the device 301; the oximetry and biometric probes are located ventral (interior) to the platform 360; and the device body 310 is located exterior to the platform 360.

In some configurations, the structural supports of platform 360 is comprised of a thin sheet or strips of plastic, metal or other flexible yet strong material (i.e., such as is manufactured via a punch and die corresponding) capable of supporting device 301 and/or device components.

In some configurations, platform 360 may enable oximetry probes and/or oximetry modules to physically attach to the oximetry probe platform interlock 388. In one example, the oximetry probe housing may contain a snap-in, lock, button, adhesive fabric, Velcro or other mechanism engaging the oximetry probe platform interlock or oximetry module platform interlock that contains the corresponding (mated) snap, lock, button, adhesive fabric, Velcro or other paired attachment mechanism.

In some configurations, platform 360 may contain snaps, locks, buttons, adhesive fabric, Velcro or other mechanisms engaging the wearable garment 380. For example, wearable platform 360 may include a metal snap (male) for engaging a corresponding metal snap (female) located on the garment 380. In another example, platform 360 may include “hook” Velcro for engaging a corresponding “loop” Velcro located on the garment 380.

FIG. 3E illustrates one embodiment of device 151 for wear on the upper torso, like a shirt or vest, in which the inner layer 120 of device 101 contains the electronics underlayment 121 of the device. The inner layer 120 comprises an elastomeric material fit snugly on the upper torso for better sensor adherence, similar to an athletic compression top, and may contain conductive fabric to represent conductive electrodes (ECG/EMG). The inner layer 120 can be constricted (i.e., via elastic draw, Velcro, etc.) to increase pressure on sensor probes at locations 114 and 115, among others. These locations are accessible by User from the external layer 112.

The outer fabric (external layer 112) will cover any part of the torso not covered by internal layer 120 (the neck, back, breast, and abdominal area) and will also provide the exterior cover the internal layer 110. External layer is long sleeved, short sleeved, or sleeveless. Areas of the shirt made only by the outer fabric will feel loose and free to add to patient comfort. Both fabric layers are washable.

The wearable device 151 may include a mechanism for securing the removable electronics underlayment 121 to the internal layer 120. The electronics underlayment 111 and external or internal layers is secured to each other and to the patient using cables, straps, Velcro or other self-adhesive material, snaps, buttons, compression garments, constrictive bands, elastomeric materials, bi-stable springs and/or a combination of the preceding.

In one embodiment, electronics underlayment 121 slides into a preformed fabric pocket that is permanently attached to an elastomeric internal layer 120, to allow User to insert the underlayment 121 manually from the top front of the device 101. This pocket (when empty) can be washed by User 130 along with the external and Internal layers.

In one embodiment, underlayment 121 attaches directly onto the internal layer 120, and has an internal device-to-skin interface that is appropriate for continuous wear. This interface is wiped clean by User 130 after removal or prior to placement.

FIG. 3E also illustrates one configuration of the components of the electronics underlayment 121. In this configuration the wearable device 301 contains a removable underlayment 121 that houses one NIRS oximetry probe 132, two PPG oximetry probes 133, one non-oximetry heart rate probe 134 (i.e., single lead ECG, green light PPG), one device body 135 (housing the logic and communications modules, and user-interface), and at least two motion-state sensors 136. Individual sensor modules is contained within the device body, or may stand alone near their corresponding probes and communicate via wire or wirelessly with device control logic. The removable underlayment 121 is a single semi-rigid unit or may include one or more anatomically shaped semi-rigid segments joined by a flexible fabric or other elastomeric material, or via inelastic joints such as hinges.

In one embodiment, the electronics underlayment 121 houses two optical PPG probes 133, peri-sternal, and at least 4 cm apart to prevent cross-contamination of light signal. In such an embodiment, the device body 135 is located on the sternum and houses the motion state sensors 136, and alarm system 320. The sternal location of the motion state sensors 136 minimizes false positives from extremity (arm/leg) movements.

The sternal location of alarm system 320 places audible and visual stimulation in-close proximity to User senses, and sternal location of tactile/haptic alarm (vibratory alert) 323 may enable tactile stimulation to clinically replicate User stimulation similar-to a sternal rub technique.

In another embodiment, the PPG oximetry probes is approximately 4 cm apart vertically to prevent cross-contamination of light signal; and both is located directly on the sternum. In other embodiments the device body 135 is in between the oximetry probes, or inferior to the probes, and is sternal or parasternal.

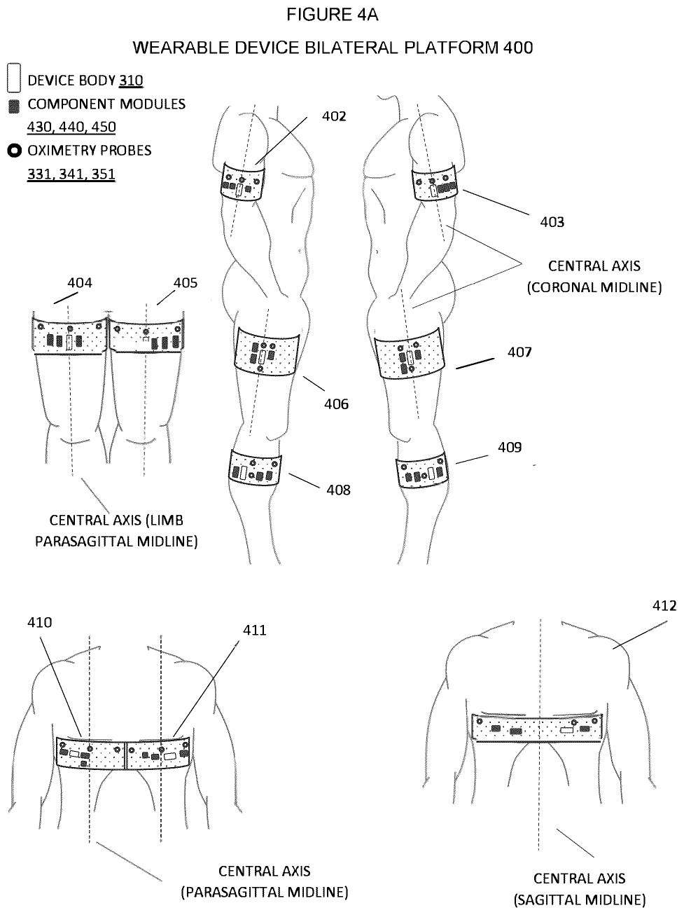

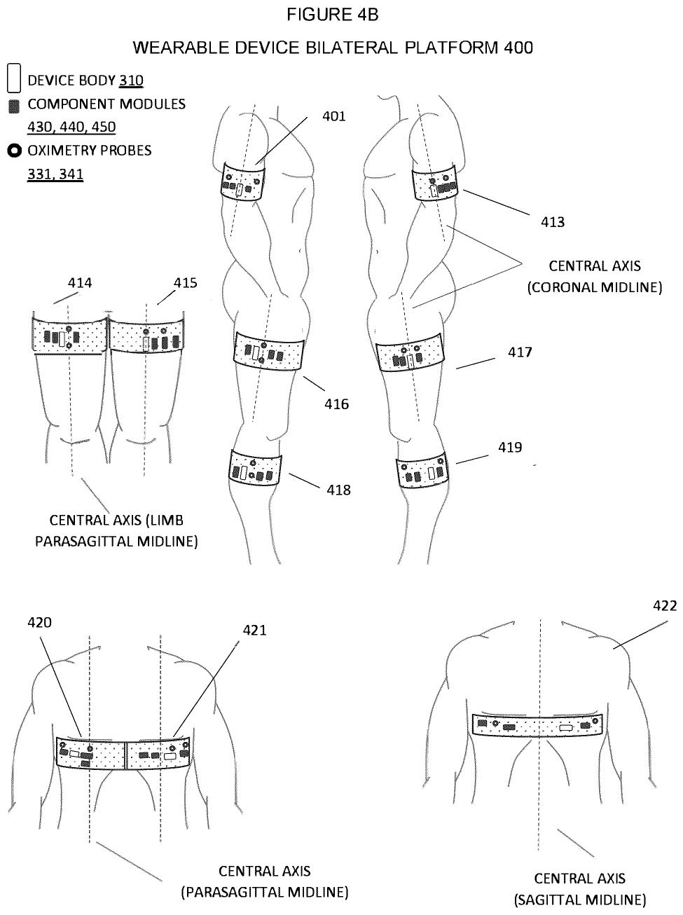

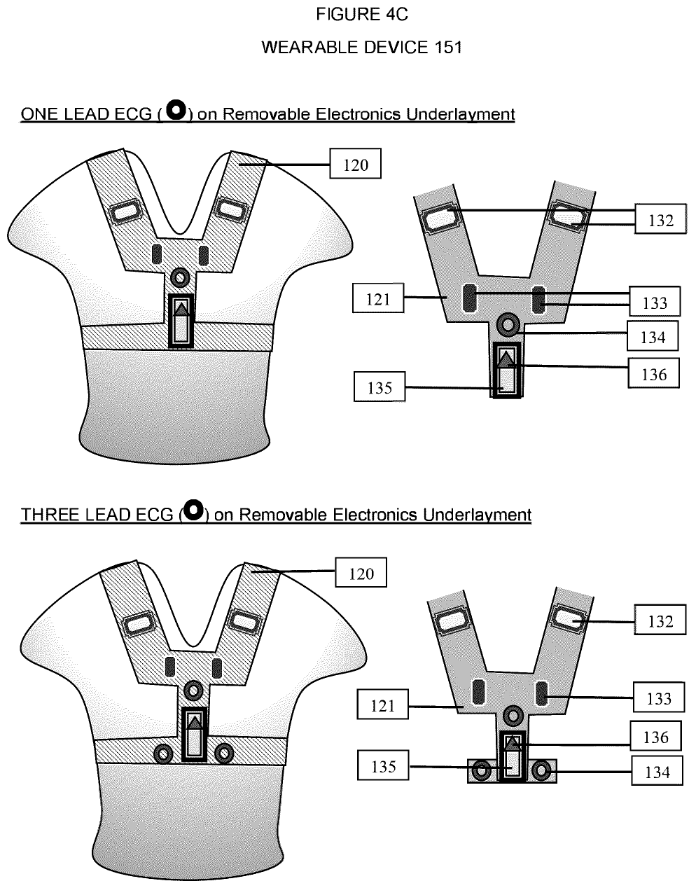

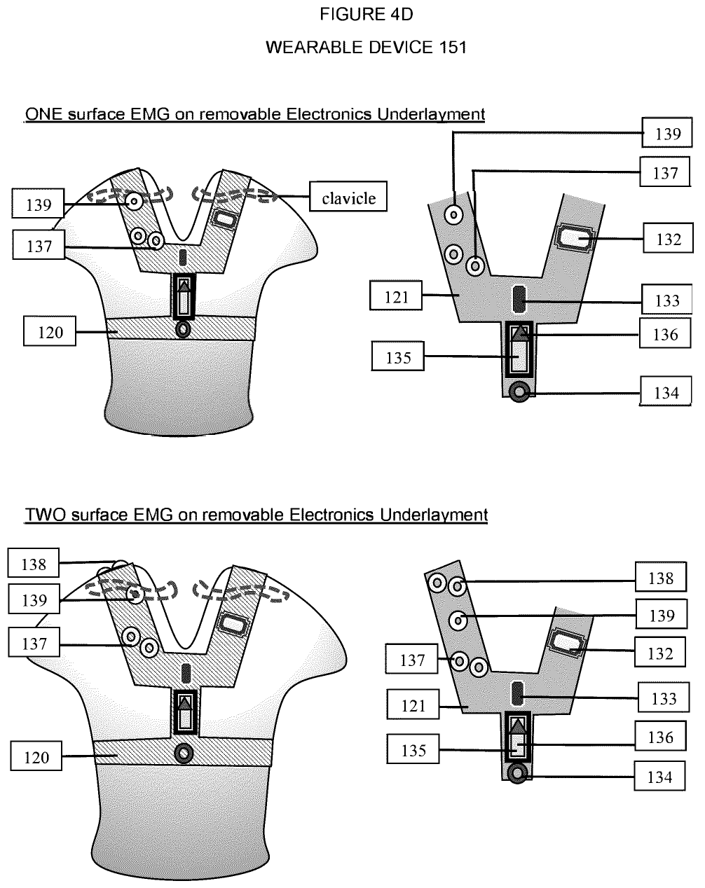

FIG. 4A illustrates a wearable device platform 400 for a wearable device 301 that enables bilateral use, (i.e., wear on both right and left sides of body). The platform 400 is configured to enable bilateral use with a single device, with the same or comparable oximetry probe locations on each side of the body (i.e., oximetry Probes will be located over comparable target tissues on right or left arms; right or left legs; and right or left sides of torso).

The platform 400 is configured to approximate the anatomical curve of the upper (proximal) limb, and oriented in relation to the anatomical shape of the limb, torso or other body location (i.e., “in-line” being located along the long-bone of the limb or “orthogonal” wrapping around the circumference of the limb or torso), enabling bi-lateral use. For example, configurations 402 and 403 orient oximetry probes “orthogonally” to the limb, wrapping around the circumference of the upper arm; and configurations 410 and 411 are oriented “orthogonally” to the torso, wrapping around its circumference. In another example, configurations 406 and 407 orient the oximetry probes “in-line” with the central axis of the limb, positioned along the length of the upper leg.

In some configurations, the platform 400 is disclosed as approximating the anatomical curves along a central axis of the limb or torso (i.e., “in-line” curving at 0-90 degrees and located along the central axis, and “orthogonal” curving at 90-180 degrees and located perpendicular to the central axis).

Platform 400 is configured to approximate the anatomical curves of a relatively planar region of the body such as the torso (i.e., device components are located “planar”, located flat along the chest, upper back, lower back, or external obliques).

Platform 400 is also oriented in relation to a central axis of body location. In some configurations, one or more oximeter probes are located on each side of the axis, enabling the platform to be suitable for bilateral use. For example, configurations 402, 406, 408 and 409 have at least one oximetry probe located on each side of the central axis of the coronal midline of the limbs. In another example, configuration 404 has at least one oximetry probe located on each side of the central axis of the parasagittal midline of the limb. In another example, configuration 411 has at least one oximetry probe located on each side of the central axis of parasagittal midline of torso. In yet another example, configuration 412 have at least one oximetry probe located on each side of the central axis of the sagittal midline of the torso.

In some configurations one or more oximeter probes are located bi-laterally (and in some cases, equilaterally) to the central-axis designated by the midline of limb; or one or more probes located on either side of the central axis designated by the midsagittal plane of torso.

In some configurations, the platform 400 may contain one or more oximeter probes that is located along the central axis of a body location, and one or more oximetry probes located laterally. For example, configurations 402, 403, 404, 405, 410, 411 and 412 have three Oximeter Probes, one located on the central-axis designated by the midline of limb, and two located bi-laterally, one on each side of the central axis.

In another example, configurations 406 and 408 have three oximeter Probes, two located on the central axis designated by the midline of limb, and one located laterally, on one side of the central axis.

The wearable device 301 is also oriented in relation to a central axis of body location, with oximeter probes located in approximately functionally similar locations on each side of the axis, measuring oximetry data from non-identical sides in close proximity to one another (i.e., two oximeter probes, each one located laterally to the central-axis designated by the midline of limb, and one oximeter probe relatively superior, relatively lateral, or relatively superior and relatively lateral to the other Oximeter Probes), enabling the platform to be suitable for bilateral use. Although the Oximeter Probes are not located in identical orientations (on right vs left limb, or right vs left Torso), they reflect similar target tissues on either side of the central axis; not identical, but functionally equivalent to one another in terms of oximetry measurements (i.e., SpO2, rSO2, pulse rate, respiratory rate).

In some configurations, the wearable device 301 may contain one or more accelerometers, gyroscopes or magnetometers to determine relative orientation of oximetry probes to target tissues on similar body locations (i.e., right limb and left limb). For example, such a wearable device 301 worn on the right limb (with oximetry Probe A located on right anterior limb and oximetry Probe B located on right posterior limb), will use gyroscopic, accelerometer or magnetometer measurements and device logic to determine the relative positions of oximetry Probes A and B when the same wearable Oximeter is worn on the left, (i.e., reversing the orientation of oximetry Probes, with Probe A now located on left posterior limb and Probe B now located on left anterior limb), enabling bilateral use of the wearable Oximeter.

In configuration 402, the wearable platform 400 is configured for wear on the right lateral upper arm (right triceps and biceps). In configuration 403, the platform 400 is configured for wear on the left lateral upper arm (left triceps and biceps). In configuration 404, the platform 400 is configured for wear on the right anterior upper leg (i.e., right thigh). In configuration 405, the platform 400 is configured for wear on the left anterior upper leg (i.e., left thigh). In configuration 406, the platform 400 is configured for wear on the right lateral upper leg (i.e., right hip). In configuration 407, the platform 400 is configured for wear on the left lateral upper leg (i.e., left hip). In configuration 408, the platform 400 is configured for wear on the right lateral lower leg (right calf). In configuration 409, the platform 400 is configured for wear on the left lateral lower leg (left calf). In configuration 410, the platform 400 is configured for wear on the right upper torso. In configuration 411, the platform 400 is configured for wear on the left upper torso. In configuration 412, the platform 400 is configured for wear on the central upper torso.

The configuration 402 shows one configuration of the wearable device 301 on User 130's upper right arm, with the device body 310 located along a central-axis of the midline of the lateral upper arm (coronal midline), device component modules 430 and 440 are located posterior to the device body 310, and component module 450 is located anterior to the device body 310. oximetry probe 331 is located posterior to the device body 310; oximetry probe 341 is located in-line with the device body 310; and oximetry probe 351 is located anterior to the device body 310.

The configuration 403 shows one configuration of the wearable device 301 on User 130's upper left arm, with the device body 310 located along a central-axis of the midline of the lateral upper arm (midline of Coronal plane), and device component modules 430, 440 and 450 are located posterior to the device body 310. oximetry probe 331 is located anterior to the device body 310; oximetry probe 341 is located in-line with the device body 310; and oximetry probe 351 is located posterior to the device body 310.

The configuration 404 shows the wearable device 301 on User 130's upper anterior right leg (along the quadriceps) or right thigh, with device body 310 located along a central-axis of the midline of the right upper leg (i.e., midline of limb or midline of Right Parasagittal plane), and device component modules 430 and 440 are located lateral to the device body 310, and device component module 450 is located medial to the device body 310. oximetry probe 331 is located lateral to the device body 310; oximetry probe 341 is located in-line to the device body 310; and oximetry probe 351 is located medial to the device body 310.

The configuration 405 shows the wearable device 301 on User 130's upper anterior left leg (along the quadriceps) or anterior left upper leg (left thigh), with device body located along a central-axis of the midline of the upper leg, (i.e., midline of limb, midline of left parasagittal plane); device component modules 430, 440 and 450 are located lateral to the device body 310; oximetry probe 331 is located medial to the device body 310; oximetry probe 341 is located in-line to the device body 310; and oximetry probe 351 is located lateral to the device body 310.

The configuration 406 shows the wearable device 301 is located on the right upper lateral leg (along the vastus lateralis) of User 130's right hip, with device body 310 is located along the midline of the lateral upper leg (midline of Coronal plane); device component modules 430 and 440 are located lateral and posterior to the device body 310, and device component module 450 is located anterior to the device body 310; oximetry probes 331 and 341 are located along the central-axis of the mid lateral upper leg (midline of Coronal plane) and oximetry probe 351 is located anterior to the device body 310.

The configuration 407 shows the wearable device 301 is located on the left upper lateral leg (along the vastus lateralis) of User 130's left hip; the device body 310 is located along the midline of the lateral upper leg (midline of Coronal plane); device component module 430 is located lateral to the device body 310, and device component modules 440 and 450 are located anterior to the device body 310; oximetry probes 331 is located posterior to the device body 310; oximetry probe 341 is located a central-axis of the midline of the lateral lower leg (Coronal midline); oximetry probes 351 is located anterior to the device body 310.

The configuration 408 shows the wearable device 301 on User 130's lower right leg, with device body 310 located a posterior to the central-axis of the midline of the lateral lower leg (Coronal midline); device component module 430 is located posterior to the device body 310; device component module 440 and 450 are located anterior to the device body 310: oximetry probes 331 and 341 is located along the central-axis of the midline of the lateral lower leg (Coronal midline); and oximetry probe 351 is located anterior to the central-axis of the midline of the lateral lower leg (Coronal midline), along the tibialis anterior, to the device body 310.

The configuration 409 shows the wearable device 301 on User 130's lower left leg, with the device Body 310 is located posterior to the central-axis of the midline of the lateral lower leg (Coronal midline); device component modules 430 and 440 are located anterior to the device body 310, and device component module 450 is located posterior to the device body 310; oximetry probe 331 is located anterior to the device body 310 (along the tibialis anterior); oximetry probes 341 is located anterior to device body 310; and oximetry probe 351 is located posterior to device body 310.

The configuration 410 shows the wearable device 301 is located on the right upper torso (along the external obliques and serratus anterior) of User 130, with the device body 310 is located inferior to the pectoralis major, lateral to the midline of the right chest wall (midline of right anterior parasagittal plane), device component modules 430 is are located laterally to device body 310; device component modules 440 and 450 are located medially to device body 310, and located superior and inferior to one another; oximetry probe 331 is located lateral to device body 310; oximetry probe 341 is located medial to the device body 310 and nearby the mid-line of right chest wall (i.e., midline of right parasagittal plane); oximetry probe 351 is located medial to the oximetry probe 341 and nearby the central axis (sagittal midline) of the anterior torso.

The configuration 411 shows the wearable device 301 is located on the left upper torso (along the external obliques and serratus anterior) of User 130, with the device body 310 located inferior to the pectoralis major and lateral to the midline of the left chest wall (midline of left parasagittal plane): device component modules 430 and 440 located medially to device body 310 and medial to to the midline of the left chest wall (midline of left parasagittal plane); device component module 450 is located laterally to device body 310; oximetry probes 351 is located medial to the device body 310 and nearby the central axis (sagittal midline) of the anterior torso; oximetry probe 341 is located along the mid-line of left chest wall (midline of left parasagittal plane); and oximetry probe 331 is located lateral to the device Body 310.

The configuration 412 shows that the wearable device 301 is located on the upper central torso. The device body 310 is located on one side of the upper central torso inferior to the pectoralis major, (i.e., along parasagittal midline); device component modules 430 and 440 are located on the right side of the upper torso, and device component module 450 is located on the left side of upper torso; oximetry probes 331 is located medial to device body 310, inferior to pectoralis major along the right external oblique or serratus anterior; oximetry probe 341 is located along the central-axis of the mid-line of left chest (i.e., midline of left parasagittal plane); and oximetry probe 351 is located lateral to device body 310, inferior to pectoralis major along the left external oblique or serratus anterior.

In other configurations, the wearable device 301 is used in other body locations, such as the lower back, upper back (trapezius), ventrodorsal gluteal (buttocks), abdomen or forearm.

FIG. 4B illustrates other configurations of wearable device platform 400 for a wearable device 301 that enables bilateral use, (i.e., wear on both right and left sides of body), with two oximetry probes 331 and 341.

In some configurations, the platform 400 may contain one or more oximeter probes that is located along the central axis of a body location, one or more oximetry probes located laterally to the central axis, or a combination thereof. For example, configurations 414, 416 and 418 have two oximeter probes located on the central axis designated by the midline of limb (coronal midline or parasagittal midline).

In another example, configurations 401, 417, and 419 have two oximeter probes located anterior and posterior to the central axis designated by the midline of limb (coronal midline). In another example, configuration 422 has two oximeter probes located lateral to the central axis designated by the midline of torso (sagittal midline).

In another example, configuration 413 has two oximeter probes, one located along the central-axis designated by the midline of limb (coronal midline), and one located posterior to the central-axis. In another example, configuration 421 has two oximeter probes located lateral to the central-axis designated by the midline of left upper-torso (left parasagittal midline).

In other configurations, the wearable device 301 is used in other body locations, such as the lower back, upper back (trapezius), ventrodorsal gluteal (buttocks), abdomen or forearm, with two oximetry probes.

FIG. 4C illustrates wearable device 151 in accordance with some embodiments. FIG. 4C illustrates one embodiment in which an electronics underlayment 121 houses two NIRS oximetry probes 132, two PPG oximetry probes 133, one non-oximetry heart rate or pulse-rate biosensor (i.e., single lead ECG, green light PPG) 134, and at least two motion state sensors 136, and a device body 135 housing all corresponding sensor modules, device logic, and communications modules.

Bilateral oximetry probes improve data collection, decrease loss of data due to loss of probe contact or focal interference, and is used to confirm critical vital signs.

FIG. 4C illustrates one embodiment of wearable device 151 in which the underlayment 121 retains the size and shape to be comfortably attached on the inside of internal layer 110, and/or fit into a custom-made fabric pocket that is attached to internal layer 110.

FIG. 4C also illustrates another embodiment of wearable device 151 in which the device 151 has three ECG leads 134. Multiple ECG leads may allow device Logic to better characterize the User's cardiac rhythm, as well as decreased number of sensor failures and false positives, by having confirming leads.

FIG. 4C also illustrates an embodiment of device 301 in which multiple ECG leads are separated by more than 3 cm allow a 2D and 3D characterization of the heart by viewing it from three locations; in this case superior, right lower, and left lower.

FIG. 4C also illustrates an embodiment of device 151 may include a removable underlayment 121, and an Internal Layer 110 that houses washable conductive fabric and is used in place of replaceable ECG probes. Washable conductive fabric will allow the Proposed device to contain multiple ECG leads. Conductive fabric will also enable an easily removable (Y-shaped) electronics underlayment to be applied directly on top of fabric.

FIG. 4D illustrates wearable device 151 in accordance with some embodiments. FIG. 4D illustrates one embodiment that includes a removable electronics underlayment 121 that houses one NIRS oximetry probe 132, one PPG oximetry probe 133, one non-oximetry heart-rate sensor (i.e., single-lead ECG or green light PPG) 134, at least two motion-state sensors 136, a device body 135 housing all corresponding sensor modules, device logic, and communications modules, and one noninvasive surface electromyography (sEMG) sensor with a pair of sEMG probes 137 are aligned with the belly of the pectoralis major muscle for optimum detection, and one sEMG probe 139 is the ground and needs to overlie bony prominences (e.g. clavicle and/or sternum) for best signal. sEMG can create a unique pattern during tonic-clonic seizures and is excellent for diagnosis of an ictal event within seconds of occurrence, making it usable as a wearable seizure monitoring method.

In one embodiment device logic may combine sEMG data and accelerometer data for increased seizure sensitivity and specificity, as both sensors create unique muscle movement profiles during an active seizure that cannot be reproduced by activities of daily living.

In another embodiment device Logic may combine heart rate (and/or rhythm) with sEMG (and/or accelerometer) for increased seizure sensitivity and specificity. Heart rate typically increases prior to a seizure at a rate discordant with nightly rest activities; device Logic can combine this increase with the specific patterns produced by sEMG and/or accelerometer to conclude if seizure is most likely occurring.

FIG. 4D also illustrates wearable device 151 in accordance with an embodiment that includes five sEMG probes to characterize one additional large muscle movement. In this embodiment the second set of sEMG probes 138 is attached posteriorly to the right upper trapezius muscle. The ground sEMG 139 (located over the clavicle) can be used for both sEMG set 137 (pectoralis major) and sEMG set 138 (upper trapezius). Increasing the number of sEMG probes may allow device Logic to better characterize the User's ictal rhythm, as well as decreased number of sensor failures and false positives, by having confirming leads.

FIG. 4D also illustrates one embodiment of wearable device 151, in which the electronics underlayment 121 retains the size and shape to be comfortably attached on the inside of internal layer 110 and/or fit into a custom-made fabric pocket that is attached to internal layer 110. The longer “arm” of the underlayment 121 is flexible to allow it to wrap over the clavicle and fall posteriorly onto the right upper trapezius.

In another embodiment of wearable device 151, the sEMG, PPG and NIRS sensor probes is reversed to allow the longer “arm” of removable underlayment 121 to fall posteriorly onto the left upper trapezius.

Another embodiment of wearable device 151 may include an electronics underlayment 121 and an internal layer 110 that houses washable conductive fabric and is used in place of replaceable sEMG probes.

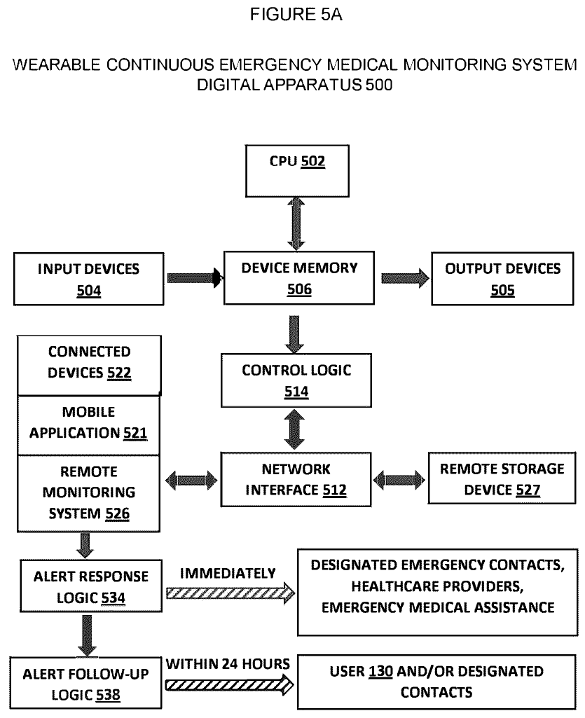

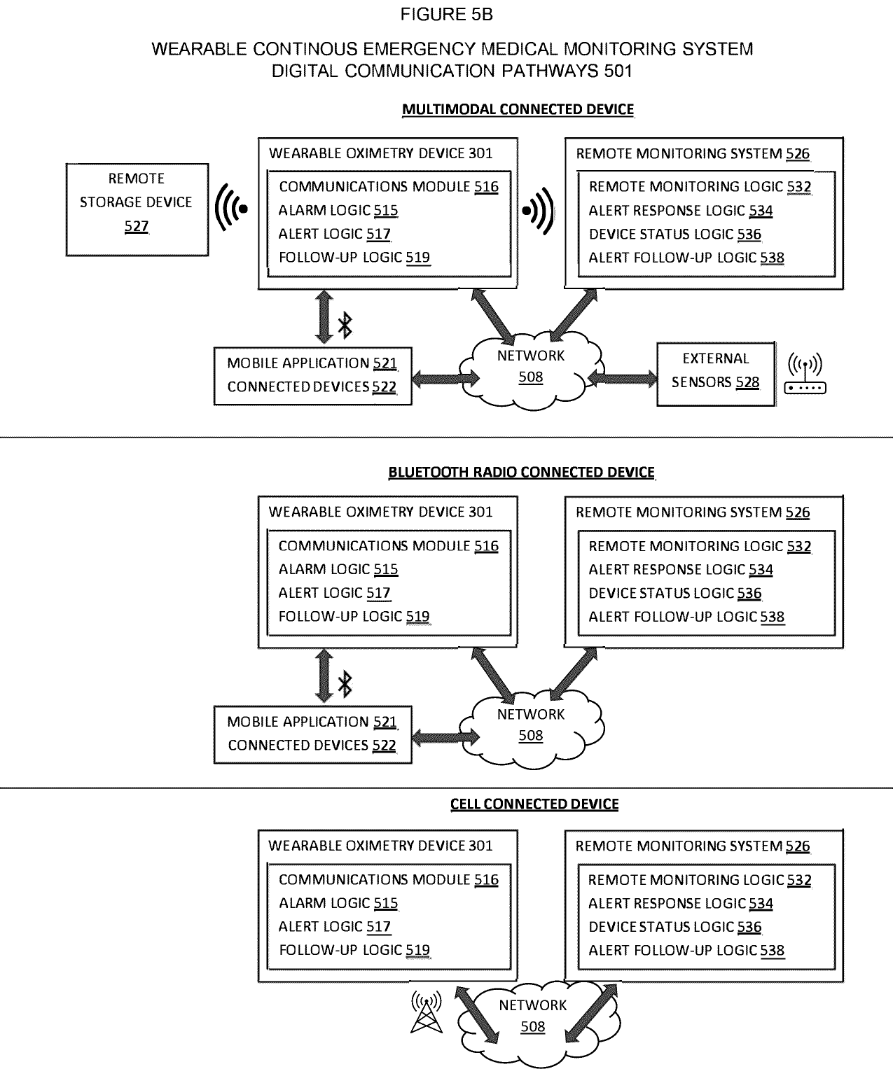

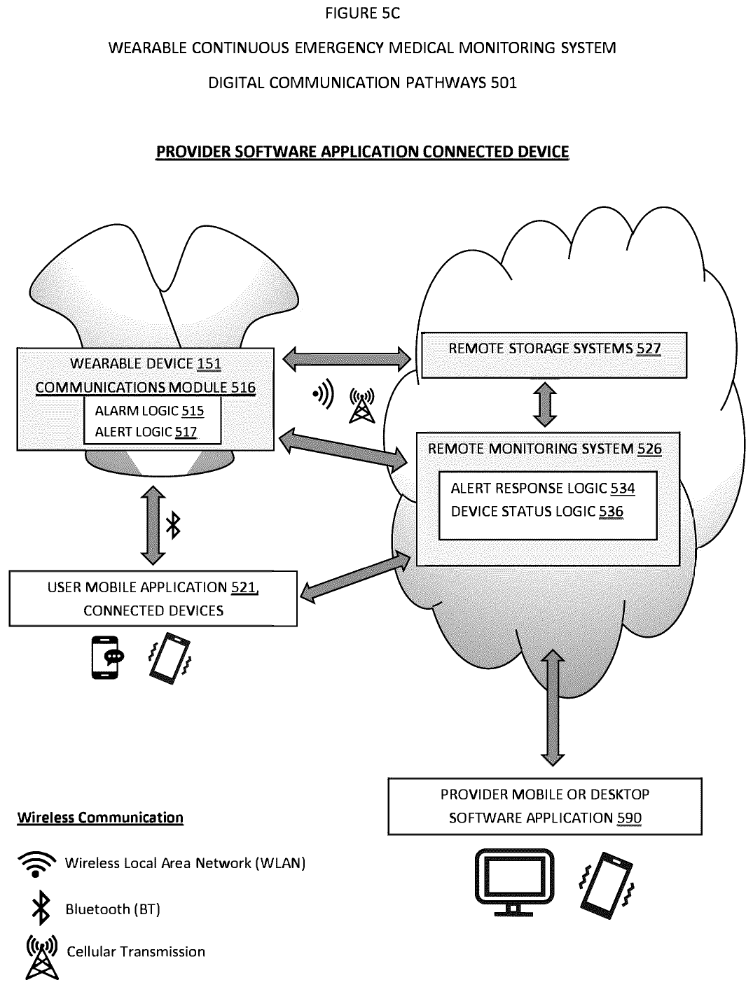

FIG. 5A illustrates the digital processing and wireless communication of the wearable oximetry System 300. FIG. 5A illustrates embodiment of a Digital Apparatus 500, to implement components and process steps of the system disclosed herein.

As depicted in FIG. 5A, input devices 504 comprise probes or transducers that convert biophysical phenomenon into machine internal signals, typically electrical, optical or magnetic. Signals is also wireless in the form of electromagnetic radiation in the radio frequency (RF), infrared or optical range. Examples of input devices 504 are optical sensors such as PPG and NIRS sensors; gravitational, inertial force or magnetic sensors such as accelerometers, gyroscopes, magnetometers and sEMG sensors; temperature sensors; electrodermal activity sensors; contact sensors which respond to touch or physical pressure from the user's skin; among other examples.

The signals from the input devices 504 are provided via various machine signal conductors (e.g., buses or network interfaces) and circuits to memory 506.

The memory 506 is typically what is known as a first or second level memory device, providing for storage (via configuration of matter or states of matter) of signals received from the input devices 504, instructions and information for controlling operation of the central processing unit (CPU) 502, and signals from cloud-based remote monitoring system 526.

In some configurations, network interface 512 will be a communication with a remote software application on a mobile application 521, connected device 522 or remote monitoring system 526. In some configurations, network interface 512 will only allow unidirectional data transmission from the device 301 to mobile application 521, connected device 522 or remote monitoring system 526, or remote storage device 527. In some configurations, network interface 512 may allow two-way communication.

Information stored in the memory 506 is typically directly accessible to the CPU 502 of the device. Signals input to the device cause the reconfiguration of the internal material/energy state of the memory 506, creating in essence a new machine configuration, influencing the behavior of the digital apparatus 500 by affecting the behavior of the CPU 502 with control signals (instructions) and data provided in conjunction with the control signals. Cloud-based remote storage device 527 may provide additional memory capability.

The CPU 502 may cause the configuration of the memory 506 to be altered by signals in remote storage device 527. The CPU 502 may alter the content of the memory 506 by signaling to a machine interface of memory 506 to alter the internal configuration, and then converted signals to the remote storage devices 527 to alter its material internal configuration. In other words, data and instructions is backed up from memory 506, which is often volatile, to remote storage devices 527, which are often non-volatile.

In some configurations, Information or signals sent to the cloud-based remote storage device 527 is subsequently removed/deleted from the memory 506. In some situations, it is desirable to only retain a minimal amount of user data in memory 506. For example, minimal user data on the device 301 may better protect user privacy. In another example, minimal user data on the device 301 may require a much smaller amount of memory 506 than would otherwise be required to store significantly larger amounts of information. However, reducing the size of memory 506 may require more frequent data uploads (i.e., once per 20 minutes instead of once per hour) or reduction in size of data uploads (i.e., transmitting limited data or pre-processing data for optimal transmission size).

Output devices 505 are transducers which convert signals received from the memory 506 into physical phenomenon such as sound (i.e., via audible devices/speakers such as audible alarm 321), or patterns of light on a machine display (i.e., via LED lights such as visual alarm 322), or vibrations (i.e., via haptic/tactile devices such as vibratory alert 323), or digital displays or software application dashboards (i.e., remote monitoring system software, electronic medical records, and digital reports), or patterns of ink or other materials on paper or other substance (i.e., printed reports). In some configurations, the output device 505 is a local storage device, such as a USB drive or laptop.

In some configurations, the output device 505 is a transducer that converts signals received (from memory 506 or CPU 502) to an activation signal or control logic signal for a drug delivery system that is communicably coupled to the output device 505.

The network interface 512 receives signals from the memory 506 and converts signal into electrical, optical, or wireless signals to other machines, typically via a machine network. The network interface 512 also receives signals from the machine network and converts them into electrical, optical, or wireless signals to the memory 506 or remote storage devices 527.

In some embodiments, the functions of the remote software application may be performed by Device Logic, including operating alarm response logic 534.

The remote software application on mobile application 521, connected device 522, and/or remote monitoring system 526 operates alert response logic 534 to send voice, text, or mobile application alerts to User 130, User 130's designated Emergency Contacts, Health Care providers, and Emergency Medical Assistance Services, including 9-1-1 and Emergency Medical Services. In some configurations, alert response logic 534 may include multiple levels of wireless alerts, indicating escalating severity of medical events (i.e., “Low”. “Medium” and “High”) reflected in escalating frequency of outreach to user and designated contact, or urgency of message content if wireless alerts. In some configurations, alert response logic 534 may respond with an appropriate alert level as provided by input from the device alert logic 317 (i.e., alert logic 317 can request a “Medium” alert or “High” alert, based upon the user's vital signs and stage of medical event). In some configurations, alert response logic 534 may determine the appropriate alert level independently from device alert logic 317 (i.e., alert response logic 534 may independently escalate the alert level from “Low” to “Medium” to “High” based upon time elapsed since initial alert signal sent from device 301). In some configurations, alert response logic 534 may include contacting Additional Medical Assistance via text or voice contact, including 9-1-1 or other monitored emergency service.

Remote monitoring system 526 operates sensor logic 536 to communicate with control logic 514 of wearable device 301, mobile application 521, connected device 522 or any combination thereof, device status logic 536 may communicate with control logic 314 to discern status of wearable device 301 and its components, such as oximetry modules 330, 340, 350; oximetry probes 331, 341 and 351; non-oximetry biometric sensor 312; biometric sensor probes 358 and 359, communications module 316, alarm cancel/reset button 324, among others.

Remote software applications on connected device 522 or remote monitoring system 526 operates alert follow-up logic 538 to send signals to a mobile application 521 on User 130's mobile phone; operates software program or script on connected device 522; and send voice, text, or mobile application alerts to User 130, mobile application 521, connected device 522, User 130's pre-designated emergency contacts, health care providers, and medical event follow-up services, including crisis response, counselling, social support and other such “wraparound” services. In some configurations, alert follow-up logic 538 may include multiple levels of wireless alerts, indicating escalating urgency of outreach after medical events (i.e., “Low”, “Medium” and “High”) reflected in escalating frequency of outreach or urgency of message content if wireless alerts following medical event.

FIG. 58 illustrates the communication pathways of wearable oximetry System 300 using wearable devices 301 connected via Bluetooth radio, WLAN radio, cellular radio, or multimodal communication mechanisms.

FIG. 5B illustrates the multiple communication pathways of wearable Emergency Medical Monitoring System 300 from the wearable device 301 to the remote software applications located on mobile applications 521, connected devices 522, or remote monitoring system 526 via the wireless communication Module 316 and Network 508. FIG. 5B also illustrates the wearable device 301 in communication with mobile application 521 and connected devices 522 via Bluetooth radio, and in communication with external/environmental Sensors 528 via the Network 508.

FIG. 5B also illustrates an embodiment in which Alarm logic 515 operates with control logic 514 to send activation signals to audible alarm 321, visual alarm 322 and tactile/haptic alarm (vibration alert) 323 in response to detected medical events. In some configurations, alarm logic 515 may include multiple levels of Alarm intensity, indicating escalating severity of medical events (i.e., “Low”, “Medium” and “High”), reflected in escalating volume or pattern of audible alarms, brightness or pattern of visual alarms, and intensity or duration of tactile alarms.

Alert logic 517 operates with control logic 514 to send signals to remote software application located on mobile application 521, connected device 522, remote monitoring system 526, or a combination thereof.

In some configurations, alert logic 517 may include multiple levels of Wireless Alerts, indicating escalating severity of medical events (i.e., “Detected,” “Low”, “Medium” and “High”). In some configurations, alert logic 517 may include device 301 contacting additional medical assistance directly via voice, text or software alert using wireless communication module 316 (i.e., emergency contacts, healthcare providers, and/or 9-1-1 or other monitored emergency service).

device follow-up logic 519 operates with control logic 514 to send signals to a mobile application 521 on User 130's mobile phone or other software program or script on Connected device 522. In some configurations, alert follow-up logic 519 may include escalating frequency of outreach or urgency of message content of voice or text outreach or prompts from mobile application 521. In some configurations, device follow-up logic 519 may contact remote monitoring system 526 or connected device 522.

Mobile Application 521 operates on User 130's phone. In some configurations, Mobile Application 521 is in communication with remote software application on a connected device 522 or remote monitoring system 526 via network 508. In some configurations, mobile application 521 is linked to device 301 via Bluetooth or other wireless communication method. In some configurations, mobile application 521 is configured to receive voice and text/SMS alerts from remote monitoring system 526, alert response logic 534, alert follow-up logic 538 or other applications.

Connected devices 522 may operate independently or in conjunction with wearable device 301, in some configurations, connected devices 522 is linked to device 301 via Bluetooth or other wireless communication method. In some configurations, connected devices 522 is configured to receive voice and text/SMS alerts from remote monitoring system 526, alert response logic 534, alert follow-up logic 538 or other applications.

FIG. 5C illustrates that wearable device 301 communicates with remote monitoring system (RMS) 526 and remote storage devices 527 via wireless local area network (WLAN) or via direct cellular transmission from device communication module 316 to network. device 301 stores raw and analyzed data in on-board memory 506. Cloud-based remote storage devices 527 may provide additional memory capability and allow prolonged storage for longitudinal health evaluation. Also, data and instructions is backed up from memory 506 (which is volatile) to remote storage device 527 (which are often non-volatile).

In one configuration, device 301 communications module 316 communicates directly to User's connected device via Bluetooth and vice versa, with no cloud network involvement, using a mobile medical application 521. In other configurations, the device 301 uses the cloud network to reach User via mobile application 521 or connected devices 522, and vice versa. Two directional wireless communication between User's connected device 522 and device 301 may involve a combination of Bluetooth, wireless local area network (WLAN), and cellular communication.

In some configurations, device communications module 516 will only allow unidirectional data transmission from the device 301 to the connected devices 522, cloud-based remote monitoring system 526, remote storage 527, to protect User privacy and to protect device 301 from external intrusion. In some configurations, communications module may allow two-way communication between device 301 and connected device 522, remote monitoring system 526, and remote storage 527, with User knowledge and acceptance (e.g., to help identify cause of error signal from device, or to remotely activate alarm if concern is present or to use previous baseline data to detect new aberrancy, etc). In the latter case, User will have the option to “Opt-Out” of bilateral communication.

In some configurations, information or signals sent to the cloud-based remote storage device 527 is subsequently removed/deleted from the memory 506. In some situations, it is desirable to only retain a minimal amount of User data in memory 506, as it may better protect User privacy if device is taken by others. In another example, minimal User data in memory 506 may require a much smaller amount of device memory than would otherwise be required to store significantly larger amounts of information, thus decreasing battery size and minimizing device design.

In one configuration of the wearable 301 and wireless communication module 516, the remote monitoring system 526 operates alert response logic 534 to send signals to a mobile application 521 on User's phone; operates software program or script on the Connected device 522; and sends voice or text message via mobile application 521 or via Bluetooth, WLAN, or cellular access to connected device 522. Remote monitoring system 526 may also notify User approved contacts (e.g. family or caregiver) by text/voice/email via direct cellular access or by notification using associated medical messaging application on family or caregiver phones). In some configurations, Alert Response Logic 534 may include escalating frequency of outreach, or urgency of message content, of voice or text or prompts from mobile application.

In one configuration of the device 301 and wireless communication module 516, the remote monitoring system 526 operates alert response logic 534 to send signals to a mobile or desktop application 590 for use by Healthcare provider; operates software program or script, and sends notification to mobile application 521, connected device 522, or sends voice or text message via cellular radio to mobile application 521, associated mobile applications for family or caregivers, or connected device 522.

The User's mobile application 521 may also display device status and user data after being retrieved from the device 301 or cloud-based remote monitoring system 526 or remote storage 527. The mobile application 521 may also enable the patient to directly contact the remote monitoring system 526 (via the application user interface) or via their phone (i.e., voice call, text) for the purposes of receiving information or additional medical services. The mobile application may allow users to record medical symptoms and provide subjective descriptions of user experience using voice, text and photos. Additionally, the mobile application may allow users to record time events for known behaviors which may induce medical events (i.e., missed medications, physical exertion, emotional distress).

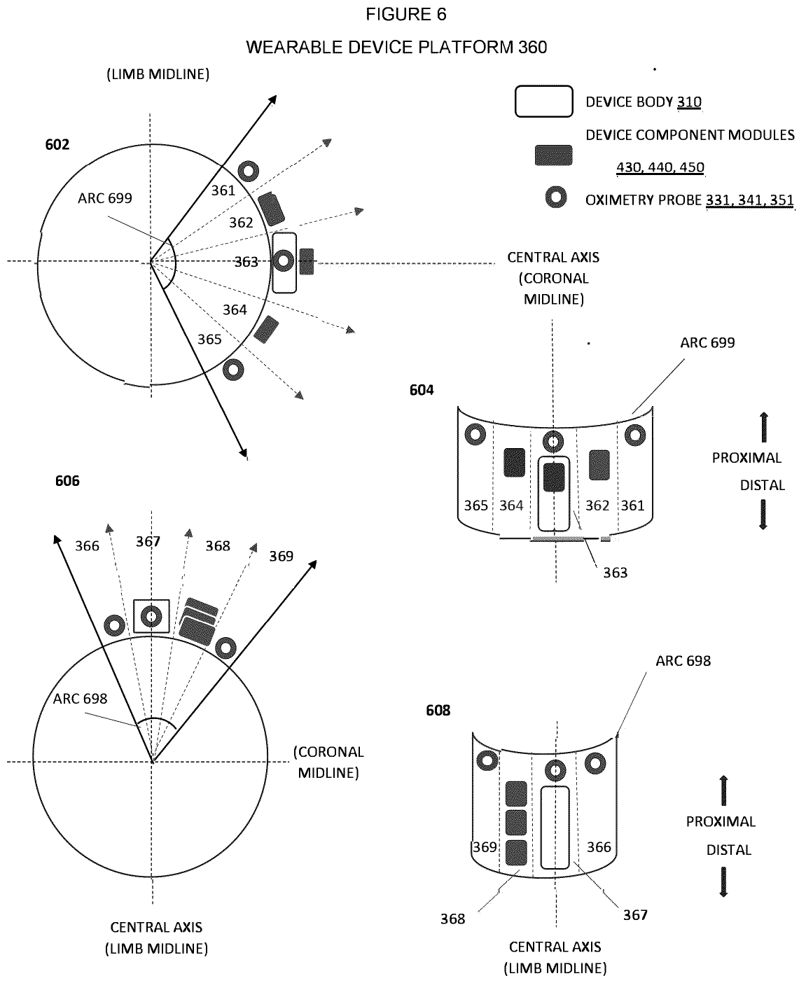

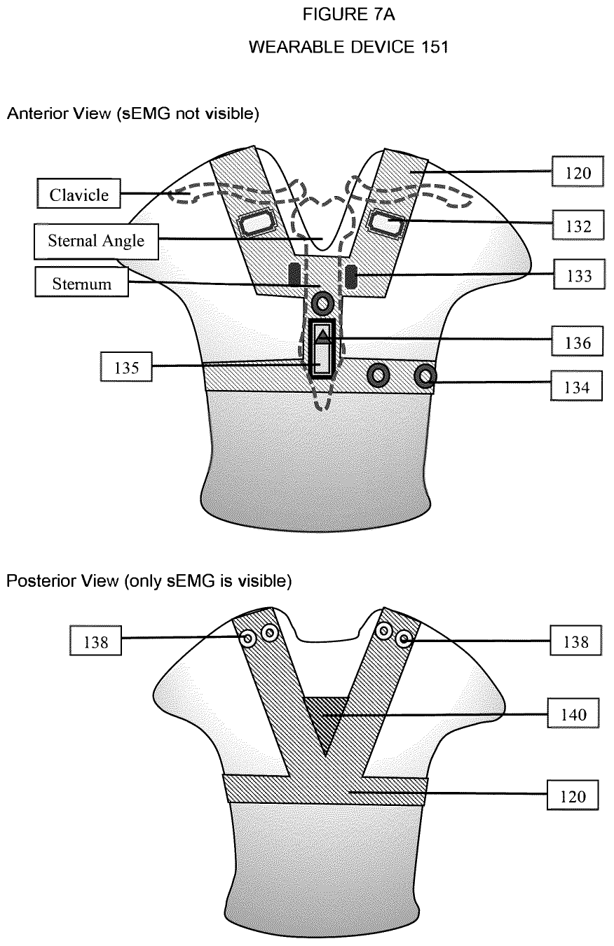

FIG. 6 depicts some embodiments of wearable device 301 and device platform 360 from a variety of Perspectives, with coronal midline and limb midline shown as a guide to device position. In Perspective 602, the platform 360 is shown from a superior/top-down view, from the shoulder proximally to the elbow. In Perspective 604, the platform 360 is shown from a side exterior view along the coronal midline. In Perspective 606, the platform 360 is shown from a superior/top-down view, from hip proximally to knee. In Perspective 608, the platform 360 is shown from a frontal exterior view along the midline of the anterior limb (thigh).

In some configurations, the wearable device platform is configured to approximate the anatomical curve along the circumference of a limb Arc 699. In some configurations, this limb is the upper arm, whose measure of an Arc is approximately <180 degrees, (i.e., measured laterally from the midline of the triceps to the midline of the biceps). Such a wearable platform is configured to the measure of an Arc of preferably 90-150 degrees, (i.e., measured laterally from a point lateral to the midline of the triceps, around the circumference of the limb, to a point lateral to the midline of the biceps), to allow the wearer the largest range of unobstructed motion. For example, in Perspective 602 the measure of the Arc 699 is approximately 120 degrees.

In some configurations, the wearable platform 360 may include a series of attached segments 361, 362, 363, 364 and 365 anatomically shaped to improve the comfort and minimize the physical profile of the device at the body location. The number of segments is generally more than two: the number of segments for Arcs measuring <180 degrees is generally 3-7. For example, in Perspective 602 there are 5 segments for an Arc measuring 120 degrees.

The anatomically shaped segments of wearable device platform is oriented in relation to the body location on which they are positioned, such that the curvature of the device Segment conforms to the underlying Target Tissue. For example, Perspective 602 and 604 depict the platform 360 comprised of segments 361, 362, 363, 364 and 365 oriented in an Arc 699 measuring approximately 120 degrees.

In another configuration, the segment of the wearable platform is configured to approximate the anatomical curve of the anterior upper leg, whose measure of an Arc is approximately <120 degrees, (i.e., measured laterally from anterior thigh to lateral thigh or hip). Such a wearable platform is configured to the measure of an Arc of preferably 45-90 degrees, (i.e., measured laterally from a point medial to the midline of the anterior thigh, to a point medial to the midline of lateral thigh) to allow the wearer to largest range of unobstructed motion. For example, in Perspective 606 the measure of the Arc 698 is approximately 70 degrees.

In some configurations, the wearable device Platform 301 may include a series of attached/linked segments 360 anatomically shaped to improve the comfort and minimize the physical profile of the device at the chosen body location (i.e., anterior thigh). The number of segments is generally more than two: the number of segments for Arcs measuring <120 degrees is generally 3-5. For example, in Perspective 606 there are 4 segments for an Arc measuring 60 degrees. For example, Perspectives 606 and 608 depict the platform 360 configured for wear on the right upper leg and comprised of Segments 366, 367, 368, and 369 oriented in an Arc 698 measuring approximately 70 degrees.

In another example, the wearable oximetry platform is configured to approximate the anatomical curve around the circumference of a limb, whose measure of an Arc is approximately <180 degrees, (i.e., measured laterally from the midline of the triceps to the midline of the biceps) using a wearable device 301 contained within a flexible unibody or “patch” form factor. Such a unibody wearable platform is configured to retain the measure of an Arc of preferably 90-150 degrees. The flexible unibody is enclosed in a wearable garment or covered in a flexible material such as silicone.

In another example, the wearable oximetry platform is configured to approximate the anatomical curve along a limb, whose measure of an Arc is approximately <120 degrees, (i.e., measured approximately from a point medial to the midline of the limb, to a point on the lateral limb, anterior to the midline of coronal plane) using device 301 contained within a flexible unibody or “patch” form factor. Such a wearable platform is configured to the measure of an Arc of preferably 60-90 degrees.

In some configurations, each segment may contain one or more device components or sensors (i.e., oximetry module, oximetry probes, device body, wireless communication module, alarm module) of device 301. For example, platform segments 361, 363 and 365 may include oximetry probes 331, 341, 351; segments 362 and 364 may include component modules 530 and 550; and segment 363 may include the device component module 540 and oximetry probe 341, collocated on segment 363 with the device body 310).

In some configurations, each segment may contain more than one device component module or oximetry probe of device 301. For example, as depicted in Perspectives 606 and 608, platform segment 368 contains device component modules 530, 540 and 550.

As illustrated in Perspective 602, in some configurations, the segment of the wearable platform is configured to enable equal weight distribution along central axis of the device 301 and laterally located device component modules and oximeter probes. For example, platform 360 distributes the weight of device body 310 approximately equally along segment 363; distributes the weight of device component modules 530, 540 and 550 approximately equally among segments 362, 363 and 364; and distributes the weight of oximetry probes 331, 341 and 351 approximately equally among segments 361, 363 and 365. In another example, platform 360 distributes the collective weight of device body, device component modules and oximetry probes bilaterally along the platform segments, such that the weight of segments 361, 362 and 363 are approximately equal to the weight of segments 363, 364 and 365.

In another configurations, a curved, semi-flexible unibody is configured to enable equal weight distribution along central axis of the device body and laterally located device component modules and oximeter probes.

The segments of the wearable platform are also configured to enable anatomically based weight distribution such that the device Segment along the central axis of the limb length supports a majority of the weight of the device body, device component modules and oximeter probes. For example, in Perspectives 602 and 604, platform 360 distributes weight of device body 310, device component module 340 and oximetry probe 341, representing a majority of device weight, along Segment 363.

The segments of the wearable platform configured to enable anatomically based weight distribution such that at least two device Platform Segments along the central axis of the limb length support most of the weight of the device Body, Oximeter Modules and Oximeter Probes. For example, in Perspectives 606 and 608, platform 360 distributes the weight of device body and device component modules, which collectively represent most of the device weight along segments 367 and 368.

In another example, a semi-flexible unibody or “patch” wearable platform is configured to enable anatomically based weight distribution such that the central axis of the lateral limb supports the device body 310, with weight of device component modules distributed evenly on each lateral side of the unibody platform.

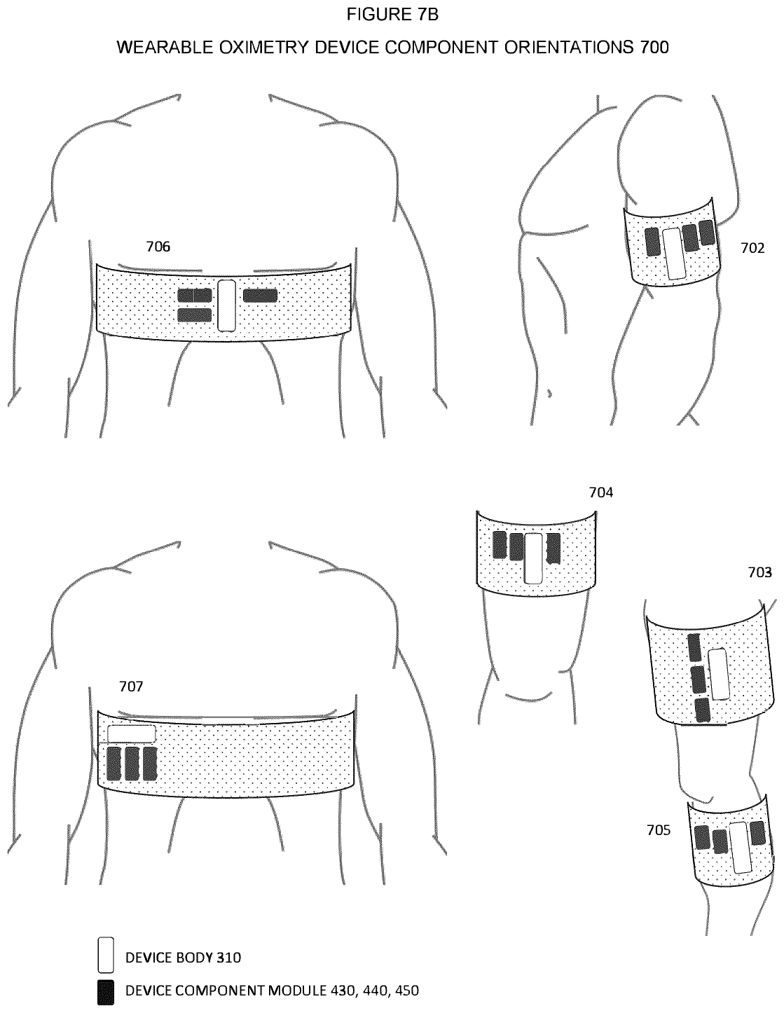

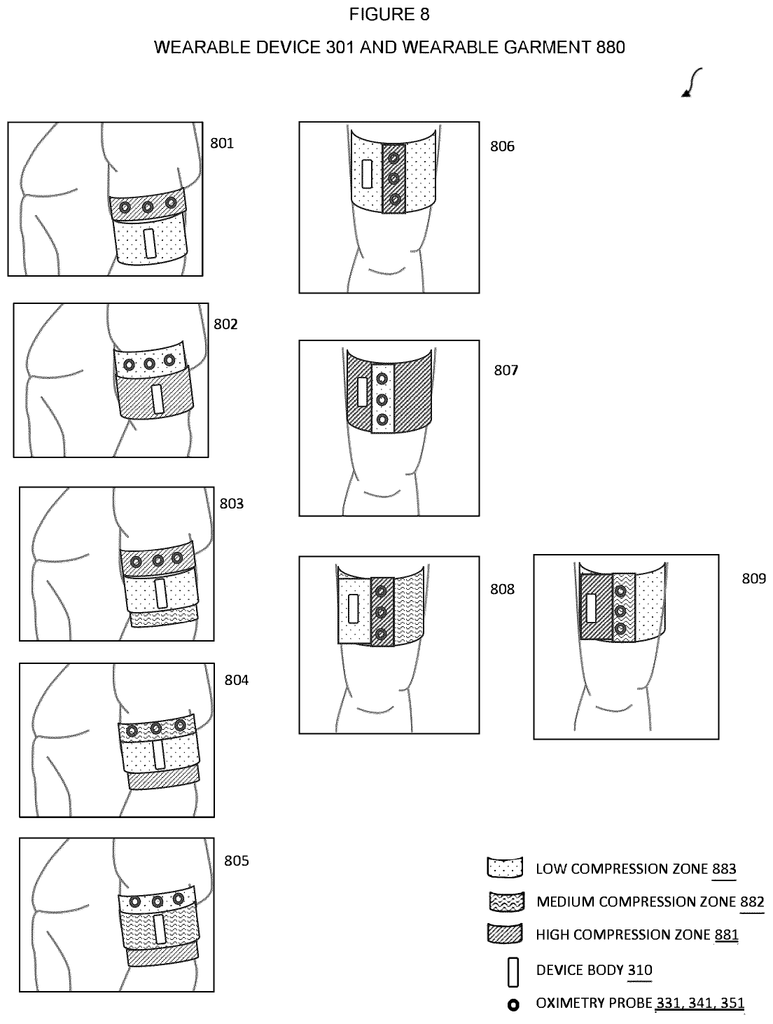

FIG. 7A illustrates another embodiment of wearable device 151 in accordance with an embodiment that includes two sub-clavicular NIRS oximetry probes 132, two parasternal PPG oximetry probes 133, three pericardia ECG leads 114 (anterior, inferior, and inferolateral), four sEMG leads 138 (2 muscle bundles: right and left trapezius), and a sternal motion state sensor 136. The bilateral sEMG leads 138 may enable interpretation of laterality of seizure or laterality of seizure onset. The location of the pericardiac ECG leads 134 may enable improved visualization of the heart during and after ictal event. The device body 135 lies mid sternum and houses the corresponding modules. Modules and probes communicate via wires or flex circuit substrate embedded in removable electronics underlayment 121. Power is on board the device body 135 and additional power may lie on underlayment 121 and/or inferior (but attached) to the Internal layer 110. If additional structural support is needed to support components, elastic internal layer 120 is added to the ventral side of device 301.

In other embodiments, device 151 may have the sEMG probes overlie both (left and right) pectoralis major muscles, or both (left and right) trapezius muscles, or a combination of one left pectoral muscle and one left trapezius muscle, or a combination of one right pectoral muscle and one right trapezius muscle, or any other combinations of thoracic anterior and/or posterior muscles.

FIG. 7B depicts selected configurations of wearable device 301 with device body 310 and device component modules in a variety of physical orientations. The wearable device platform is disclosed herein as approximating the anatomical curvature of the body, whose components are oriented in relation to one another based on a specific axis of the printed circuit boards (PCB), (i.e., long and short axis). Some configurations (such as depicted in Orientations 702, 703, 704 and 705) is disclosed herein as device component modules 530, 540 and 550 oriented in parallel with device body 510 (i.e., all components are oriented along their long axis). Some configurations, such as depicted in Orientations 706 and 707, are disclosed herein as device component modules 530, 540 and 550 perpendicular to device body 310 (i.e., device component modules are oriented perpendicularly to the long axis of device body). Some configurations are disclosed herein as device body 310 being oriented perpendicularly compared to at least one device component module and parallel with at least one device component module.

In some configurations, the wearable device platform approximates the anatomical curvature of the body, whose components are oriented in relation to the long bone of the limb (i.e., “in-line” being located along the long bone, or “orthogonal” being located perpendicular to the long bone, wrapping around the circumference of the limb). For example, Orientations 702 and 704 are disclosed herein as approximating the anatomical curvature around the circumference of the upper limb, with device body 310 and device component modules 530 and 540 wrapping around the upper arm. In another example, Orientations 703 and 705 are disclosed herein as approximating the anatomical curvature around the Leg, with device body 310 and device component modules 530, 540 and 550 oriented in-line along the long bone of the leg.

The wearable device platform is also disclosed herein as approximating the anatomical curvature of the body oriented in relation to a central axis, located along the anterior/posterior coronal plane of the body (i.e., central axis of coronal plane along deltoid midline designating anterior and posterior deltoid; central axis of coronal plane along intercostal midline designating anterior or posterior intercostal muscles; central axis of coronal plane along lateral upper leg midline designating anterior vs posterior muscles). For example, Orientations 702, 703 and 705 are shown with device body 310 oriented along the midline of the Coronal Plane.

The wearable device platform is also disclosed herein as approximating the anatomical curvature of the body oriented in relation to the limb axis (i.e., medial vs lateral upper leg, medial vs lateral upper arm). For example, Physical Orientation 704 is shown with device body 310 oriented along the midline of the upper leg (i.e., midline of the anterior thigh).

In some configurations, the wearable device platform 9 is also disclosed herein as approximating the anatomical curvature of the body oriented in relation to the midsagittal plane of the torso (i.e., sternum or xyphoid process designating right vs left anterior upper torso). For example, Orientation 706 is shown with device body 310 oriented along the midsagittal plane of the upper torso (i.e., on or near sternum and/or xyphoid process).

In some configurations, the wearable device platform is also disclosed herein as approximating the anatomical curvature of the body oriented in relation to the parasagittal plane of the torso (i.e., approximately midline of pectoralis major on left and right, dividing each side into medial vs lateral anterior upper torso). For example, Orientation 707 is shown with device body 310 oriented along the right parasagittal plane of the upper torso.

In some configurations, the wearable device platform is also disclosed herein approximating the anatomical curvature of the body, whose components are oriented in relation to the xyphoid process (i.e., “in-line” being located anterior or superior the xyphoid process), or “orthogonal” being located laterally to the xyphoid process and wrapping around the circumference of the torso). For example, Orientation 706 is shown with device body 310 oriented along the xyphoid process of the upper torso (i.e., aligned with the midsagittal plane of the torso).

The wearable device platform is also disclosed herein as a series of flexible, connected platform segments containing PCBs oriented in relation to one another along a positive z-axis (i.e., three-dimensional) configuration. In some configurations, the PCB orientation is a triangle or trapezoidal configuration where at least one of the PCB planar surfaces is oriented in a planar fashion to the body location.

In some configurations with 3 components containing rectangular PCBs, the planar PCB has the same z-axis angle of orientation to both the non-planar PCBs (i.e., an Isosceles triangle). In some configurations, to achieve a minimized physical profile for the device, the planar PCB z-axis angle of orientation to the non-planar PCBs is substantially less than 60 degrees and the z-axis orientation of the non-planar PCBs to one another is substantially greater than 60 degrees. For example, 3 PCBs is orientated along a z-axis orientation, where one of the PCBs is planar and oriented at 30 degrees to each non-planar PCB and the non-planar PCBs are oriented at 120 degrees to each other (i.e., oriented as a 120-30-30-degree Isosceles triangle along the z-axis).

In some configurations with 3 PCBs, at least one of the PCBs z-axis orientations to another PCB is approximately 90 degrees (i.e., a right triangle). In some configurations, to achieve a minimized physical profile for the device, the planar and non-planar PCBs are oriented at 90 degrees. For example, three PCBs is orientated along a z-axis orientation, where one of the PCBs is planar and oriented at 90 degrees to one non-planar PCB and 30 degrees to the other non-planar PCBs (i.e., oriented as a 30-60-90-degree triangle along the z-axis).

In some configurations, the planar PCB in closest proximity to the body, as measured along the z-axis, is oriented to both the non-planar PCBs at the same interior angle of substantially less than 90 degrees, (i.e., an Isosceles trapezoid). For example, 4 PCBs, oriented as two planar and two non-planar relative to the body, is orientated to one another along a z-axis, where the planar PCB in closest proximity to the body location, as measured along z-axis, is oriented to both the non-planar PCBs at 45 identical degrees interior angles, and where the planar PCB in farthest proximity to the body location, as measured along the z-axis, is oriented to both non-planar PCBs at 135 degrees interior angles (i.e., oriented as a 135-135-45-45 degree Isosceles trapezoid along the x-axis.

In some configurations, to achieve a minimized physical profile for the device, the planar PCB in closest proximity to the body, as measured along the z-axis, is oriented to the non-planar PCBs at interior angles of substantially less than 90 degrees, and where the two interior angles are not identical (i.e., an acute trapezoid). For example, 4 PCBs, oriented as two planar and two non-planar relative to the body, is orientated to one another along a z-axis, where the planar PCB in closest proximity to the body location, as measured along z-axis, is oriented to one non-planar PCBs at 60-degree interior angle and another non-planar PCB at 45 degrees interior angle (i.e., oriented as a 135-45-120-60-degree acute trapezoid along the z-axis.

FIG. 8 shows various configurations of the wearable device 301 in wearable garments for the upper limb with multiple compression zones.

In some configurations, the wearable oximetry device Platform is designed as a continuous band, in which the User 130 positions the band by inserting the body location through the proximal end of the band until it is in position over the desired Target Tissue, prior to securing it to the body.

In some configurations, the wearable device platform 360 is designed as a discontinuous band (or “cuff”), in which the User 130 positions the band by inserting the body location through the opening in the discontinuous band. In some configurations, the platform 360 or wearable garment 380 may enable the device to maintain a semi-flexible arc, approximating the anatomical shape of the chosen body location, to enable earlier placement by the user.

In some configurations, the compression garment 880 may provide a physically separated device location that may attach to body via adhesive backing, simple straps, constriction straps, within a garment pocket, or a combination thereof. For example, the device platform may include discrete compression zones for discrete device components, including device body 310, device component modules 530, 540 and 550, oximetry probes 331, 341 and 351, and non-oximeter biometric probes 358 and 359.

The wearable device platform is passively constricted (i.e., elastic), manually constricted (i.e., ratchet closure), or mechanically constricted (i.e., via springs or motors in the device). In some configurations, the wearable device platform will only utilize a single device closure mechanism on compression garment 880. In other configurations, the wearable device platform will include device closure mechanisms on either size of compression garment 880.

The platform 360 may secure the oximeter probes to the wearer using a passive restraint system such as an adhesive fabric; a strap or belt manually secured by a ratchet, buckle, or Velcro closure; a strap comprising an elastomeric material; a “clamp” or “clamshell” design; or a bi-stable spring, or combinations of these, among other methods. For example, a proximal (superior) device closure may secure the oximeter probes by an adjustable elastomeric material and adjustable length Velcro straps and closures to enable comfortable continuous compression to the target tissue.

In another example, the oximeter probes are located on an inelastic or low-elasticity material, connected on either side to elastomeric material with one or more adjustable length straps and Velcro closures to enable constant or near-constant distance to be maintained between oximeter probes above the target tissue.

The wearable device platform may also secure the oximeter probes by a different mechanism than securing the device body and/or device component modules. For example, the oximeter probes is secured with a strap and ratchet proximal device closure mechanism, and device body is secured by an elastomeric band distal device closure mechanism.

The wearable device platform is also disclosed herein as approximating the anatomical curvature of the body, whose components are oriented in relation to one another based on suitability for use in a wearable garment or other attachment mechanism, containing different “compression zones” with relatively greater or less compression along the transverse plane of the limb.

The wearable device platform 360 and compression garment 880 may contain different “compression zones.” For example, as shown in Configurations 801 and 802, in some configurations, with relatively greater compression along the proximal upper limb and relatively less compression along the distal upper limb, or relatively less compression along the proximal upper limb and relatively greater compression along the distal upper limb.

The wearable device platform 360 and compression garment 880 may contain at least two different “compression zones.” For example, as shown in Configurations 801, 802, 806 and 807, in some configurations, in which the device body and device component modules are in a distinct compression zone and the oximeter probes are located in another distinct compression zone.

In some configurations, the compression garment 880 containing wearable device 301 may positioned along the limb or torso, where compression zone 881 creates relatively greater compression force (i.e., pressure experienced by the skin) than compression zone 883. For example, as shown in Configuration 801, compression zone 881 is located on the proximal side of the compression garment 880, and compression zone 883 is located on the distal side of the compression garment 880. In some configurations, compression zone 881 may contain the oximeter probes, and compression zone 883 may contain the device body and device component modules. In some configurations, compression zone 881 may contain one or more proximal closure mechanisms, and compression zone 883 may contain one or more distal closure mechanisms.

In another example, as shown in Configuration 802, compression zone 881 is located on the distal side of the garment 880, and compression zone 883 is located on the proximal side of the garment 880. In some configurations, zone 883 may contain oximeter probes, and zone 881 may contain the device body and device component modules. In some configurations, zone 883 may contain one or more proximal closure mechanisms, and zone 881 may contain one or more distal closure mechanisms.

In some configurations, the compression garment 880 containing wearable device 301 is positioned along the limb or torso, where zone 881 creates relatively greater compression force (i.e., pressure experienced by the skin of the wearer) than zone 883. In some configurations this differential compression is caused by a 2-layer garment, with the layer in closest proximity to the skin exerting a higher compression force than the layer in closest proximity to the outside of the garment.

In some configurations, the garment 880 may enable different compression zones for device platform 360 or one or more device platform segments. In some configurations, the compression garment may include an inelastic or low-elasticity platform containing the oximeter probes.

In some configurations, zone 881 may contain oximeter probes, and zone 883 may contain the device body and device component modules. For example, as shown in Configuration 806, zone 881 is located mid-thigh on the garment 880 and may contain oximetry probes, and zone 883 is located on the medial and lateral side of the garment 880, and may contain device body.

In another example, as shown in Configuration 807, compression zone 883 is located mid-leg of garment 880 and compression zone 881 is located medial and lateral to zone 883. In some configurations, zone 883 may contain oximeter probes and zone 881 may contain the device body and device component modules.

In other configurations, the garment 880 may contain three or more different compression zones, in which the device body and device component modules, oximeter probes, and proximal and distal device closure mechanisms are each located in distinct compression zones.

In some configurations, the garment 880 containing device 301 is worn on the limb or torso, positioned along the central axis of the coronal plane, orthogonal to the long bone of the Limb, where zone 881 creates relatively greater compression force (i.e., pressure experienced by the skin of the wearer) than zones 882 and 883, and where zone 882 creates relatively greater compression force than zone 883.

In some configurations this differential compression is caused by a 3-layer garment, with the layer in closest proximity to the skin exerting a higher compression force than the layer in closest proximity to the outside of the garment. In some configurations, the compression garment may include an inelastic or low-elasticity platform containing the Oximeter Modules or Probes. For example, as shown in Configuration 803, compression zone 881 is located on the proximal side of the garment 880, and compression zone 882 is located on the distal side of the garment 880, and zone 883 is located in the middle of garment 880.

In some configurations, compression zone 881 may contain oximeter probes, and compression zone 883 may contain the device body and device component modules. In some configurations, zone 881 may contain one or more proximal closure mechanisms, and zone 882 may contain one or more distal closure mechanisms.

In another example, as shown in Configuration 804, compression zone 881 is located on the distal side of the garment 880, and compression zone 882 is located on the proximal side of the garment 880, and compression zone 883 is in the middle of garment 880. In some configurations, zone 882 may contain oximeter probes, and zone 883 may contain the device body and device component modules, in some configurations, zone 881 may contain one or more distal closure mechanisms, and zone 882 may contain one or more proximal closure mechanisms.

In another example, as shown in Configuration 805, compression zone 881 is located on the distal side of the garment 880, and compression zone 882 is located mid-garment of the garment 880, and compression zone 883 in located on the proximal side of garment 880. In some configurations, zone 883 may contain oximeter probes, and zone 882 may contain the device body and device component modules. In some configurations, zone 881 may contain one or more distal closure mechanisms, and zone 883 may contain one or more proximal closure mechanisms.

In some configurations, the garment 880 containing wearable device 301 is worn on the upper limb, positioned along the central axis of the coronal plane, in-line to the long bone of the Limb, wherein zone 881 creates relatively greater compression force (i.e., pressure experienced by the skin of the wearer) than zones 882 and 883, and where zone 882 creates relatively greater compression force than zone 883. For example, as shown in Configuration 808 (i.e., left upper anterior leg/thigh), zone 881 is located on the anterior midline of garment 880, and zone 882 is located on the lateral side of the garment 880, and zone 883 is located medially on garment 880. In some configurations, zone 881 may contain oximeter probes, and zone 883 may contain the device body and device component modules.

In another example, as shown in Configuration 809, compression zone 881 is located on the medial side of the garment 880, and compression zone 882 is located on mid-line of the garment 880, and compression zone 883 is located laterally on garment 880, In some configurations, zone 882 may contain oximeter probes, and zone 881 may contain the device body and device component modules.

The wearable device 301 is secured to the patient using an “active” restraint (i.e., restraints that respond to changes in tissue conformation, such as caused by movement, without any additional action being taken by User 130) such as a spring, bi-stable spring or elastomeric band, or a “passive” restraint (i.e., restraints that do not respond to changes in tissue conformation without any additional action being taken by User 130), such as adhesive, a strap or belt secured by a ratchet or buckle, a strap comprising an elastomeric material, a “clamp” or “clamshell” design, or combinations of the preceding active and passive systems.

The wearable device 301 is also secured to the limb of a patient using a combination of passive and active restraints. In some configurations, a passive restraint (i.e., a strap tightened by a ratchet mechanism) and an active restraint system (i.e., an elastomeric band or bi-stable spring) is located on either size of the compression garment 880. For example, as shown in Configuration 802, passive restraint is located on the proximal side of garment 880 and active restraint is located on the distal side of the garment. In this situation, the wearable device 301 is configured for wear on the upper limb.

In another example, as shown in Configuration 801, passive restraint is located on the distal side of garment 880 and active restraint is located on the proximal side of the garment. In this situation, the device 301 is configured for wear on the upper or lower limb.

The passive restraint may also contain a manually engaged sizing mechanism to allow the patient to apply or remove the wearable device 301, or re-size the device platform for comfort. The sizing mechanism may consist of a wearable band sizing ratchet buckle and a wearable band sizing ratchet strap whose band engages wearable band sizing ratchet strap locking pins, that may enable the ratchet to advance or reverse, tightening or loosening the strap, respectively. For the initial placement and sizing of the band, the patient may place the wearable band on their upper arm with the ratchet strap at maximum length, depress locking/release pins, then advance the sizing strap forward to tighten against ratchet buckle until the device is comfortable.

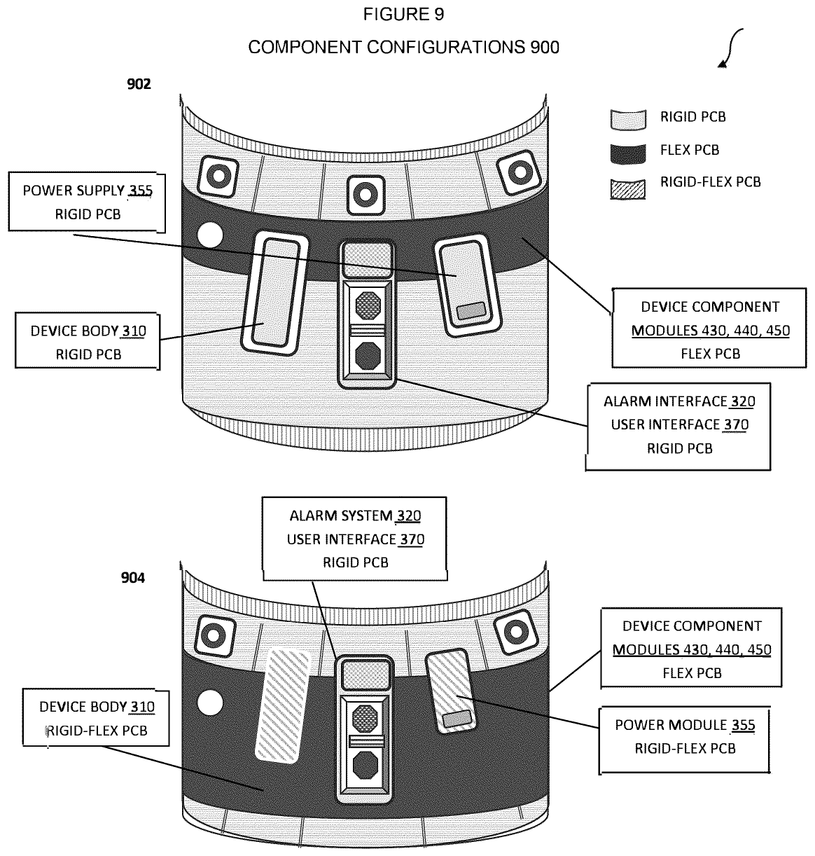

FIG. 9 shows configurations of wearable device 301 using various combinations of flexible printed circuit boards (F−PCB), rigid-flexible printed circuit boards (RF−PCB), or a combination of F−PCB and traditional rigid printed circuit boards (R+FPCB) to approximate the anatomical curvature of the body.

Rigid PCBs are defined as a solid substrate, inflexible multi-layer PCBs. Flex PCBs defined as flexible substrate, multi-layer PCBs. Rigid-Flex PCBs defined as a combination of rigid and flexible PCBs directly coupled to one another. Rigid+Flex PCBs are defined as a rigid PCB in direct communication (i.e., tension connector or solder) with a flexible PCB.

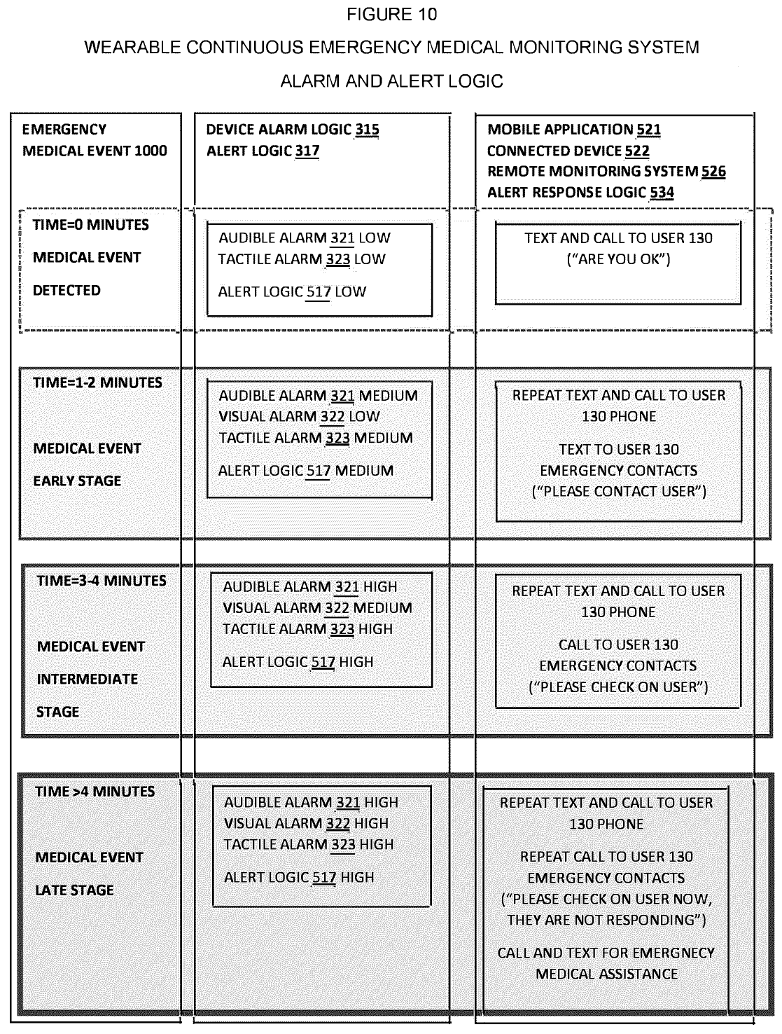

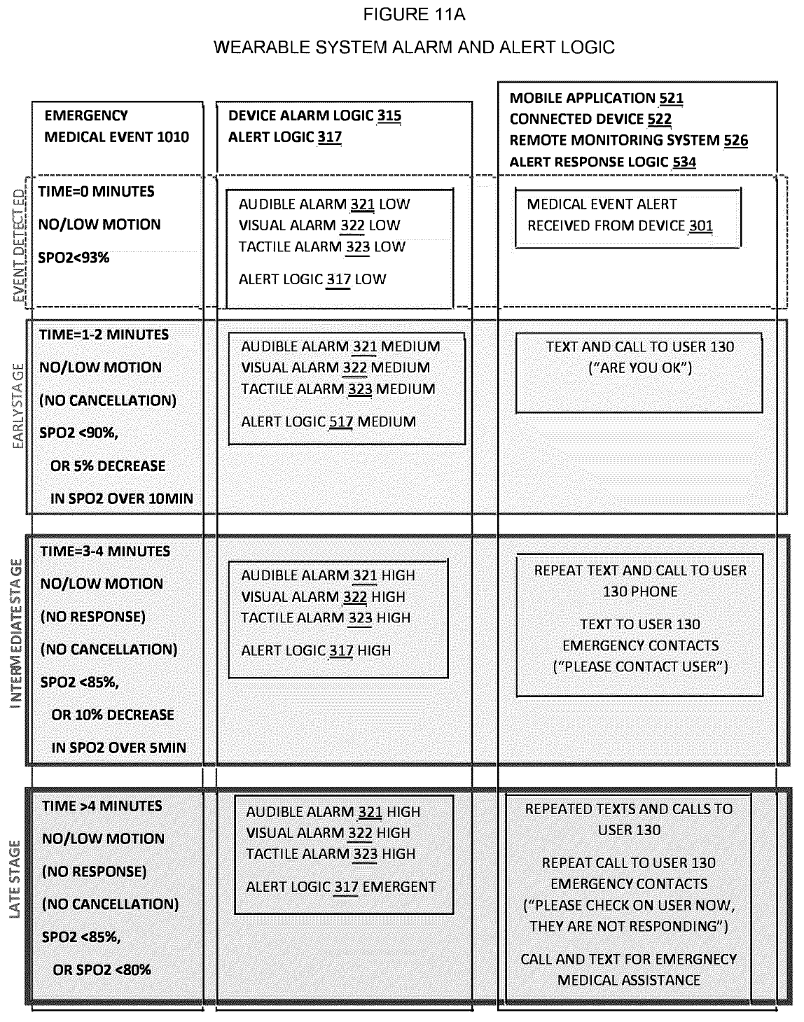

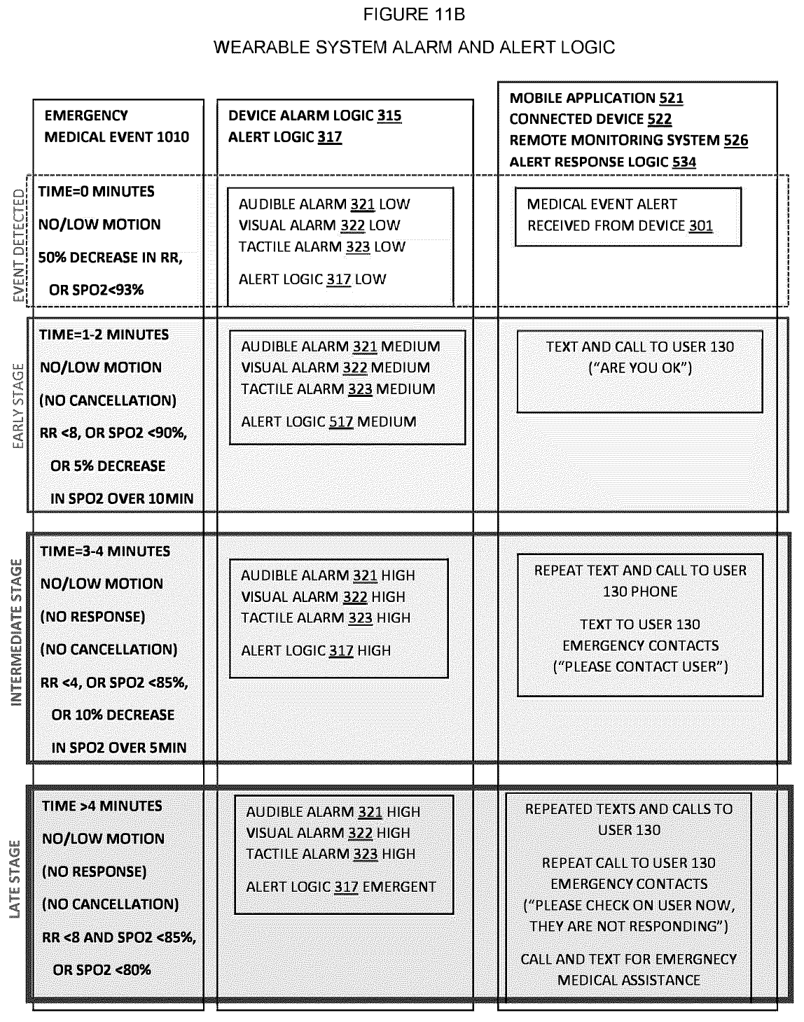

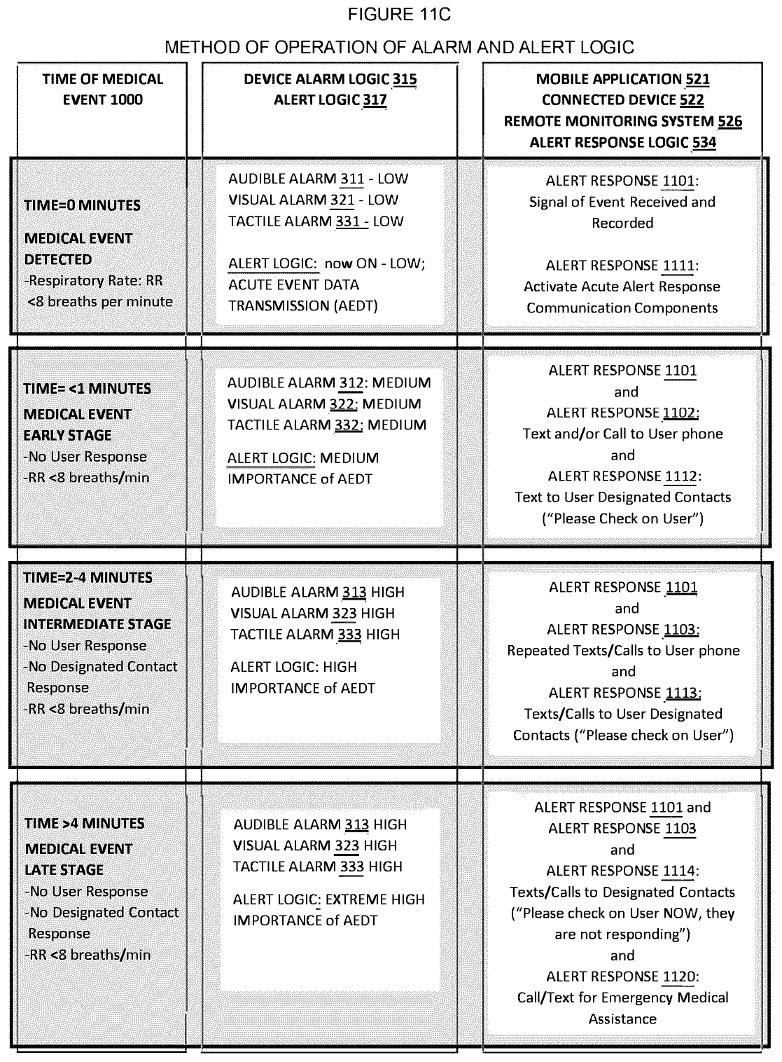

The wearable device 301 can utilize R-PCB, F−PCB, RF−PCB, or R+FPCB for devices positioned in any of the previously disclosed herein body locations (FIGS. 1A and 1B), component orientations (FIGS. 7A and 7B), in conjunction with disclosed herein device platforms (FIGS. 3C, 3D and 3E) and garments (FIG. 8), including configurations designed for bilateral use (FIG. 4), and wearable configurations along a segmented arc (FIG. 6). The choice of R-PCB, F−PCB, RF−PCB, or R+FPCB does not impact the device logic, control or communication systems disclosed herein (FIG. 2, FIG. 5, FIG. 10 and FIG. 11).

The wearable device 301 may utilize a mixture of R-PCB, F−PCB, RF−PCB, or R+FPCB for device body and device component modules. In some configurations, the device 301 may utilize R-PCB for device body and F−PCB for device component modules. For example, as shown in Design 902, device 301 may comprise RF+PCBs; including device body 310; and alarm system 320, user interface 370; and power supply 355 as Rigid-PCBs in communication with a Flexible PCB containing device component modules. Such a configuration may allow Rigid PCBs to be utilized for complex components (i.e., device body contains CPU, memory and may contain communication module, component status indicators, among other components), and components which require manual (physical) interaction with User 130 (i.e., alarm cancellation button).

In another example, as shown in Design 904, wearable device 301 may include device body 310 and power module as RF−PCBs; in communication with a F−PCB containing device component modules; and alarm system 320 and user interface 370 as R-PCBs. Such a configuration may allow RF−PCBs to be utilized for complex components whose size is minimized through the use of fewer connectors (i.e., device body 310 using direct connections to user interface 370) and allow R-PCBs to be utilized for components which require manual (physical) interaction with User 130 (i.e., alarm cancellation button).

FIG. 10 depicts a potential emergency medical event being detected by wearable Emergency Medical Monitoring System 300, with control logic 314 of device 301 using device alarm logic 515 and device alert logic 517; and remote software application located on a mobile application 521, connected device 522, and/or remote monitoring system 526 using alert response logic 534.

Control logic 514 comprises logic configured to operate the audible alarms, visual alarms and tactile alarms when a medical event is detected. The control logic monitors information from the oximetry probes, and at least one non-oximetry probe or external environmental sensor, to detect specific vital sign conditions. The control logic 314 may attempt to alarm the user or bystanders by engaging audible alarm 321, visual alarm 322, or tactile/haptic alarm (vibratory alert) 323 on wearable device 301 (as shown in FIG. 3A). If the alarm(s) are not cancelled via alarm cancellation button, and the skin contact sensors indicate that the device has not been removed by the wearer, the control logic 314 may also communicate an alert signal to remote software applications on mobile application 521, connected devices 522, or remote monitoring system 526 using the device communications module 316.

Following an alarm, the control logic 314 may also communicate to the remote software application located on a mobile application 521, connected device 522, or remote monitoring system 526 via the network, using the device communications module 316. The remote software application comprises a remote monitoring logic 532, alert response logic 534, sensor logic 536, and alert follow-up logic 538 (to contact User 130 within 24 hours of emergency medical event). The remote software application may engage alert response logic 534 to call or text the wearer's listed emergency contacts, health care providers or emergency services if appropriate. In some configurations, the alert response logic 534 may attempt to contact the wearer or bystanders by engaging audible alarms on the device 301 via the communications module 316. During the 24 hours following the medical event, the remote software application may utilize alert follow-up logic 538 to contact the wearer or designated contacts via voice, or text, or mobile application.

Alarm logic 517 is configured to signal audible alarm 321 to produce a series of tones in response to Medical Events. The series of tones produced by Audible Alarm 321 is configured to correspond to ISI/IEC 60601-1-8 standards for alarms in medical equipment. For example, as shown in FIG. 10, audible alarm 321 is configured to produce a series of tones associated with a “Low” Alarm setting upon detection of an emergency medical event; to produce a series of tones associated with a “Medium” Alarm setting approximately 60 seconds following detection of an emergency medical event and no user response; and to produce a series of tones associated with a “High” Alarm setting approximately 180 seconds following an emergency medical event. Each stage of Alert (i.e., Low, Medium, High) lasts approximately 60-120 seconds to allow for sufficient time for alarm cancellation.

In some configurations, the audible alarm 321 may also produce a series of tones outside of IEC standards. For example, the volume or frequency (or pitch) of the audible alarm may oscillate for the purpose of gaining attention of User 130 to determine if they are in need of medical assistance or drawing attention of bystanders or medical responders that User 130 is in need of medical assistance.

Alarm logic 315 is configured to signal visual alarm 322 to produce a series of lights. In some configurations, the visible alarm 322 may also produce a series of lights for the purpose of gaining attention of User 130 to determine if they are in need of medical assistance or drawing attention of bystanders or medical responders that User 130 is in need of medical assistance. For example, as shown in FIG. 10, visual alarm 322 is configured to produce a series of lights associated with a “Low” alarm setting approximately 60 seconds following detection of an emergency medical event 1000; to produce a series of brighter or blinking lights associated with a “Medium” alarm approximately 120 seconds following detection of a medical event 1000: and to produce a series of lights of blinking and brighter associated with a “High” alarm setting, approximately 180 seconds following a Medical Event 1000. Each stage of alarm or alert (i.e., Low. Medium, High) lasts approximately 60 seconds to allow for sufficient time for alarm cancellation.

Alarm logic 315 is configured to signal tactile/haptic alarm (vibratory alert) 323 to produce a series of lights. In some configurations, the tactile/haptic alarm 323 may also produce a series of vibrations, haptic pulses, or other tactile mechanisms for the purpose of gaining attention of the user 130 to determine if they are in need of medical assistance. For example, as shown in FIG. 10, alarm 323 is configured to produce a series of low intensity or short duration vibrations associated with a “Low” Alarm setting upon detection of an emergency medical event 1000; to produce a series of greater intensity or longer duration vibrations associated with a “Medium” alarm approximately 120 seconds following detection of an emergency medical event 1000; and to produce a series of high intensity vibrations or extended duration associated with a “High” alarm setting approximately 180 seconds following a Medical Event 1000.

The device communication module 316 is operable to contact another device capable of wireless communication (such as a patient's phone) and engage the device in a useful operation (such as initiating or utilizing functionality of a mobile app; communicating via SMS/text to an external counterparty, such as a clinic, hospital, emergency medical services, or call center).

The wearable device 301 is configured to communicate the detection of an emergency medical event to a remote software application located in a mobile application 521, on a connected device 522, or a remote monitoring system 526 by way of the wireless communications module 316 and network 508. The alert response logic 534 may receive the activation event signal comprising information about the wearable device 301 and the detection of the emergency medical event 1000. The alert response logic 534 is configured to initiate a response for the medical event, such as calling or texting medical staff, or emergency contacts crisis counselling services, or other support staff, or in some cases calling 911.

In some configurations, the remote software application on a mobile medical application 521, on a connected device 522, on a remote monitoring system 526, or remote storage device 527 may utilize the sensor logic 536 to build a profile of the user's health to improve determinations of possible emergent medical conditions. In some instances, software application may identify a medical event before the control logic 314 and communicate the information to the remote monitoring system 526. The sensor logic 536 may also receive information from the at least one non-oximeter biometric sensor 312 such as a no-motion or low-motion state or increased temperature activity. The alert response logic 534 is configured to initiate a response in advance of the medical event, such as calling or texting wearer, or emergency contacts, or medical staff, or crisis counselling services, or other support staff, or emergency medical services, if necessary.

Another aspect of the wearable device 301 is that control logic 314 and associated firmware is capable of interpreting multiple signal inputs, such as vital signs, device battery conditions and external environmental measurements. The electronic processor is in communication with one or more “on-board,” “local,” or “remote” sensors, or a combination of sensors (such as an on-board SpO2 monitor, smartwatch heart rate monitor, and/or in-home respiratory rate monitor) for the purpose of monitoring a medical disease state or physiologic condition which may require intervention. Monitoring of multiple signal inputs is advantageous in diagnosing or confirming the severity of an emergent medical condition (such as monitoring both the heart rate and respiratory rate of patient experiencing an allergic reaction).

In some configurations, wearable Emergency Medical Monitoring System 300 is utilized to monitor specific medical conditions, such as potential opioid overdose. Control logic 314 may monitor multiple vital signs, such as blood oxygen (SpO2) and respiration (RR) rate, utilizing both value thresholds and value trends for diagnosing or confirming Medical Event of Opioid Overdose. For example, Control Logic 314 may monitor multiple vital signs and risk biomarkers as they relate to opioid overdose, including motion-state sensors that detect a no-motion or low-motion state, and oximetry sensors that detect a decreased blood oxygen level and/or decreased respiration rate, indicating a potential emergency medical event.

Wearable System 300 is configured to detect and identify specific emergency medical events, such as opioid-overdose or other pharmacologically induced respiratory depression, seizure, post-ictal respiratory distress, severe allergic reaction, organophosphate exposure, breakthrough pain, acute anxiety, and panic attack or agitation.

FIG. 11A illustrates one configuration of a wearable System 300 detecting the emergency medical event 1010 of potential opioid-overdose or other pharmacologically induced respiratory depression through evaluation of motion-state risk biomarkers (no-motion or low-motion state) and oxygenation (SpO2).

In some embodiments, oxygenation (SpO2) for activation of alarms may be lower than shown in FIG. 11A. For example, patients with COPD or other medical conditions which impact lung function and oxygenation may require use of an oxygenation threshold wherein if SpO2 is less than 88%, it activates “Low” level alarms.

In some embodiments, device alarm logic 315 may activate alarms based upon either an oxygenation threshold (i.e. less than 90% SpO2), or a change in oxygenation (i.e. decrease of 5% SpO2 over 10 minute period), or a combination thereof.

As shown, device alarm logic 315 detects a potentially emergent medical event if motion-state sensors indicate a no-motion/low-motion state and oxygenation (SpO2) is less than 93%, and subsequently activates a series of “Low” level alarms and wireless alert, device alarm logic 315 and wireless alert logic 317 activate a “Medium” level alarm and alert if motion-state sensors continue to show a no-motion/low-motion state, and SpO2 is less than 90%, or if SpO2 is less than 95% and has decreased by 5% or more over a 10-minute period, device alarm logic 315 and alert logic 317 activate a “High” level alarm and alert if motion-sensors continue to show no motion-state change, SpO2 is less than 85% or if SpO2 is less than 90% and has decreased by 5% or more over a 10-minute period, and/or no alarm cancellation by User. Alarm logic 315 and alert logic 317 activate an “Emergent” level alarm and alert SpO2 is less than 85%, or SpO2 is less than 80% and has decreased by 10% or more over a 10-minute period.

In some embodiments, respiration rate (RR) for activation of alarms may be lower than shown in FIG. 11B. For example, patients with COPD or other medical conditions which impact lung function and oxygenation may require use of a respiration threshold wherein if RR is less than 8 bpm, it activates “Low” level alarms.

FIG. 11B illustrates one configuration of a wearable System 300 detecting the emergency medical event 1010 of potential opioid-overdose or other pharmacologically induced respiratory depression through evaluation of motion-state risk biomarkers (no-motion or low-motion state), oxygenation (SpO2) and respiration rate (RR).

In some embodiments, device alarm logic 315 may activate alarms based upon either a RR threshold (i.e. less than 8 bpm), or a change in oxygenation (i.e. decrease of 30% RR over 10 minute period), or a combination thereof.

) As shown, device Alarm Logic 315 detects an emergency medical event if motion-state sensors indicate a no-motion or low-motion state, Respiration Rate (RR) decreases by 50% over a 3-minute period (i.e., from 10 breaths per minute to 5 breaths per minute), or Oxygenation (SpO2) is less than 93%; and subsequently activates a “Low” level series of alarms and alerts, device alarm logic 315 and alert logic 317 activate a “Medium” level series of alarms and alerts if motion-state sensors continue to show a no-motion or low-motion state, and RR is less than 8 breaths per minute or if SpO2 is less than 90%, or if SpO2 is less than 95% and has decreased by 5% or more over a 10-minute period, device alarm logic 315 and alert logic 317 activate a “High” level series of alarms and alerts if motion-state sensors show no change, and RR is less than 8 breaths per minute or SpO2 is less than 85% or if SpO2 is less than 90% has decreased by 5% or more over a 10-minute period, and/or no alarm cancellation by User. Alarm logic 315 and alert logic 317 activate an “Emergent” level series of alarms and alerts if patient continues to be unresponsive, RR is less than 4 breaths per minute, or RR is less than 8 and SpO2 is less than 85%, or SpO2 is less than 80%.

FIG. 11C an emergency medical event occurring over 4 minutes. In row one, at time of detection of Event 1000, alarm logic 315 may use a combination of low alarm signals and alert logic 317 will notify remote software application on mobile application 521, connected device 522, or remote monitoring system 526. Remote software application will then activate alert response logic 534, that event is received, and data will be acutely recorded and prepared for transmission. Remote software application will also activate alert response 1111, to ready communication components for acute notification. In row two, if emergency medical event 1000 persists and User has not responded to low alarms, Alarm Logic 315 will intensify to medium alarm signals, and alert logic 317 will send a medium-importance alert to remote software application. Remote software application will then activate Alert Response 1101, Alert Response 1102 (contact User via text, phone, or in-app message) and Alert Response 1112 (text pre-designated contacts to check on User status/safety). In row three, if emergency medical event 1000 persists and User and Designated Contacts have not responded to medium alarms, alarm logic 315 will intensify to High alarm signals, and alert logic will send a high-importance alert to remote software application. Remote software application will then activate alert response 1101, alert response 1103 (repeatedly text, call, or in-app message User), and alert response 1113 (call Designated Contact(s) to check on User status/safety). In row four, if emergency medical event 1000 persists and User and Designated Contacts have not responded to high alarms, alarm logic 315 will remain at High alarm, and alert logic 317 will send an extremely high-importance alert 1104 to remote software application. Remote software application will then activate alert response 1101, alert response 1103 (repeatedly contact User), and alert response 1114 (Repeatedly text and call Designated Contact(s) to check on User). At this stage alert response logic 534 may also call/text Emergency Medical Personnel as User appears to be in grave danger.

In one embodiment of Method of Operation of device 301, when alarm logic 315 has not been successful in notifying the User, and alert logic 317 with alert response logic 534 have not been successful in contacting User nor Designated Contact(s), and control logic 314 may also activate a communicably coupled wearable drug delivery system to deliver pharmacologic intervention in attempt to abort medical event or prolong time to decline while Emergency Medical Services arrive. Said drug delivery system is co-located on thoracic region, or elsewhere (e.g upper arm or leg). Control logic 314 may communicate to said drug delivery system via wires, or wirelessly via Bluetooth or wireless local area network (WLAN).

In another configuration, oximetry System 300 is utilized to monitor and evaluate specific medical conditions, such as respiratory depression due to concomitant use of opiates with substances which may induce or amplify respiratory depression. Control logic 314 may monitor multiple vital signs such as blood oxygen and respiration rate, utilizing both value thresholds and value trends for diagnosing or confirming overdose due to a combination of opioids and other pharmaceuticals (such as benzodiazepines) which may depress respiratory drive over time, (rather than a rapid cessation of respiratory drive as seen with higher potency opioids). For example, control logic 314 may identify a trend of decreasing respiratory drive over time a (i.e., RR decrease of 50% over 60 minutes) or persistently decreasing blood oxygen level (i.e., SpO2 decrease of 10% over 60 minutes), and alarm and alert for a potential emergency medical event.

In another configuration, oximetry System 300 is configured to assist patients at risk of potentially unanticipated, rapid opioid overdose, (such as may result from ingestion of unanticipatedly high-potency or contaminated opioids). Control logic 314 may monitor multiple vital signs such as blood oxygen and respiration rate utilizing both value thresholds and value trends for diagnosing or confirming opioid overdose with rapid decline (rather than a gradual slowing of respiratory drive which may occur with lower potency opioids). For example, control logic 314 may identify an abrupt decrease in respiratory rate (i.e., SpO2 decrease of 10% over 3 minutes) and alarm and alert for detection of a potentially life-threatening respiratory depression.

In another configuration, wearable System 300 is configured to assist patients at risk for alcohol or opioid withdrawal. For example, the control logic 314 may detect and utilize increased heart rate, respiration rate, blood pressure, temp, EDA activity, or other non-oximetry biometric sensor activity, or combination thereof, to determine withdrawal distress.

In another configuration, wearable System 300 is configured to assist patients at risk for an acute allergic reaction. For example, the Control Logic 314 may detect an increased heart rate, respiration rate, EDA and sudden decrease in blood pressure, identifying this event in User 130.

In another configuration, wearable System 300 is configured to assist patients at risk of exposure to organophosphate pesticides or chemical weapon nerve agents. For example, the Control Logic 314 may detect an increased heart rate and changes in respiration rate, and the at least one non-oximetry biometric sensor or environmental sensor may detect organophosphate presence, (or the control logic 314 may receive communication of organophosphate exposure from remote monitoring system 526 or another environmental sensor 528 sensor using network 508).

In another configuration, wearable System 300 is configured to assist patients at risk of acute anxiety and panic attacks. For example, the control logic 314 may detect acute and rapid increases in blood pressure, heart rate, respiration rate, and increased EDA.

In another configuration, wearable System 300 is configured to assist patients at risk of acute agitation. For example, the control logic 314 may detect increased blood pressure, heart rate, and respiration rate, and a motion-state sensor may detect increased locomotor activity.

In another configuration, wearable oximetry System 300 is configured to assist patients at risk of breakthrough pain. For example, the control Logic 314 may detect increased blood pressure, heart rate, respiration rate, change in heart rate variability (HRV), or increased EDA activity.

In another configuration, wearable oximetry device 300 is configured to assist patients at risk of asthma attack or COPD acute exacerbation. For example, the control logic 314 may detect a motion-state sensor indicating a low-motion state, increased blood pressure, increased respiration rate, and decreased blood oxygen.

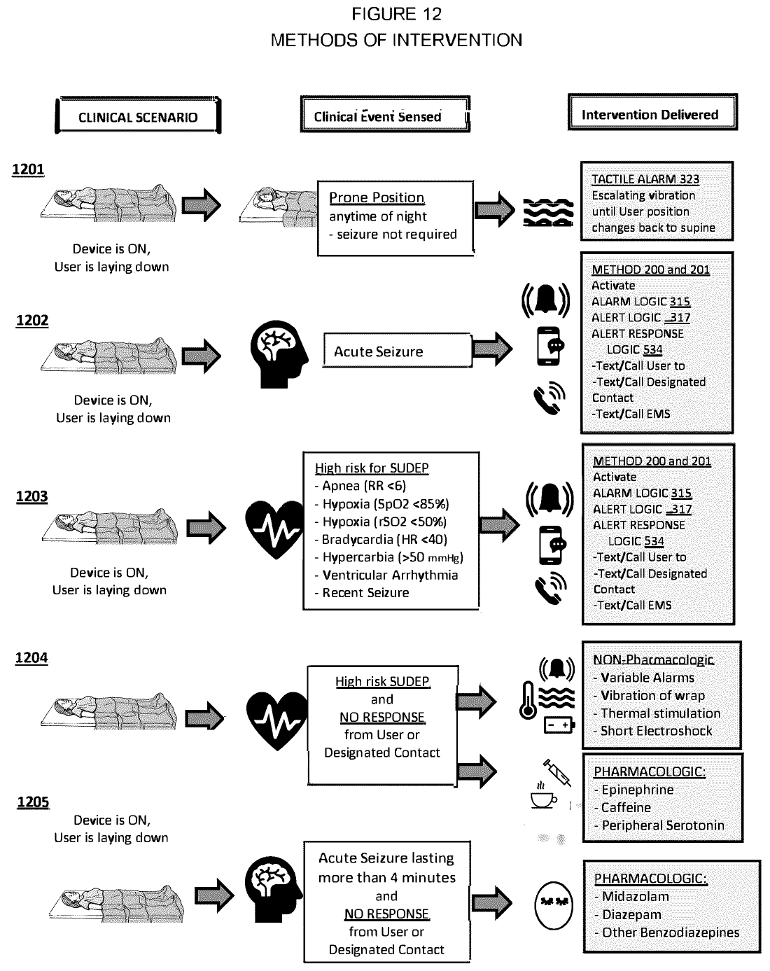

FIG. 12 illustrates methods of possible interventions in accordance with one or more embodiments of wearable device 301, device control logic minimizes false positives by being active when User is resting, either laying down or reclining <45 degrees from flat. Position can be determined by sternal motion-state sensors that can also determine if User is supine, left lateral, prone, or right lateral.

Clinical scenario 1201 depicts that the wearable device 301 may detect and intervene when the patient is in a prone sleeping position. Thus, if the patient is flat (assuming rest/sleep) and then turns into a prone position (a risk factor for SUDEP), the disclosed device may physically stimulate the patient (e.g., with vibration or low volume audible alarm) to cause them to turn to the side or supine position. The device is utilized as a training technique to encourage supine sleeping. By decreasing the duration of each prone event at night, and having persistent negative feedback over many nights, the device may decrease the total nocturnal time spent prone, which may decrease the incidence of SUDEP.

In one embodiment of the wearable device 301, the stimulating intervention is placed on the sternum, where noxious stimuli are more likely to be perceived and cause a response (the bone prominences are more sensitive locations compared to thicker, soft tissue such as muscle).

Clinical scenario 1202 depicts that the wearable device 301 may detect and intervene when the patient is having an acute seizure. Data from the motion-state sensors may show that the body is in a flat or reclining position, assuming rest/sleep. During rest, rhythmic movements that may cause seizure artifacts (exercise, tooth brushing) are much less likely. When resting, a patient's heart rate is also usually less than 100 beats per minute. Thus, if the control logic 314 detects a sudden increase in heart rate and subsequent persistent and rhythmic movement in a flat patient, seizure prediction is much more accurate compared to either of these events alone. This multisensory approach to clinical deduction makes the wearable device 301 suitable as a medical device for people with epilepsy or other seizure disorders.

In one embodiment to detect a seizure, wearable device 301 may use data from motion-state sensors to show flat position; data from PPG, NIRS, and/or HR- or PR sensors to show 50% rise in heart rate during rest; and sustained rhythmic sEMG activity for over 10 seconds. When considered together, device logic 314 may conclude that an acute seizure event is likely. By only alerting designated contacts when all three sensors are activated (flat position, abnormal HR, positive sEMG activity), late-night awakenings is minimized.

In another embodiment, the wearable device 301 may record when only sEMG sensors or only HR or PR-sensors identify potential medical events, thus recording smaller non-life-threatening events (i.e., possible, but not probable seizure) for the patient, family, and physician to review in daytime hours.

Clinical scenario 1202 illustrates possible interventions that is delivered when an acute seizure is detected. The wearable Proposed device 100 uses a novel, multi-step notification based on the emergency of the event and User preference. When seizure threshold has been reached, the wearable device 301 will activate alarm logic 315 to stimulate patient, and alert logic 317 to notify remote software application on mobile medical application 521, connected device 522, and/or remote monitoring system 526 which is capable of contacting User-designated Contacts. If there is no response from User and/or User-designated Contacts, the WPD will escalate alarms and alerts to User, User-designated Contacts, and eventually contact Emergency Medical Services in attempts to obtain help.

The User may also configure the device to send a text to User-Designated Contacts when a transient seizure has been identified (<30 sec); this is optional because nocturnal notifications are disruptive events and these seizures are normally self-limiting, but family may still want to check on User. However, if concerning vital signs are detected (signifying complications), a phone call is sent to User as well as Designated Contacts along with a simultaneous auditory alarm to awaken both patient and family.

Clinical scenario 1203 illustrates that the wearable Proposed device may detect and intervene when the User has changes in vital signs that increase the User's SUDEP risk. Generalized seizures are at risk for causing the brain to “restart,” also known as Post Ictal Global EEG Slowing, during which time breathing may become irregular and/or stop, causing a dangerous drop in oxygenation that may lead to death if unaddressed. Irregular brain activity also causes irregular cardiac rhythms, some of which is lethal such as ventricular tachycardia or ventricular fibrillation.

To determine SUDEP risk, the wearable device 301 may monitor for hypopnea (decreased breathing) or apnea (stopped breathing); hypoxia as measured by both PPG and NIRS; life threatening arrhythmia such as bradycardia (low heart rate or large heart pauses), tachycardia (atrial or ventricular tachycardia), ventricular fibrillation; and proximity of vital sign aberrancy to recent ictal event.

In one embodiment, the wearable device 301 will use device alarm and alert logic to alarm/alert when either one, or a combination of one or more, of the following aberrancies are detected: respiratory rate less than 6 breaths per minute (normal is 12-20 breaths/min), pulse oxygenation SpO2 less than 85% (normal is 91-100%), regional oxygenation rSO2 less than 50% (normal 60-90%), heart rate less than 40 beats per minute (normal is 60 beats/min).

In some embodiments, the wearable device 301 will use device logic to alarm/alert when either one, or a combination of one or more, of the following arrhythmias are detected, regardless of other sensors: cardiac pause (no heart beat) over 10 seconds regardless of return to rhythm, supraventricular tachycardia >180 beats per minute (normal is less than 100 beats/min), supraventricular arrhythmia/fibrillation over 15 seconds, ventricular tachycardia over 15 seconds, and/or ventricular arrhythmia/fibrillation over 15 seconds. At least 5-15 seconds is required for control logic to confirm data is not artifact.

Clinical scenario 1203 illustrates possible interventions that is delivered when a high risk for SUDEP event is detected, device 301 uses a novel, multi-step notification based on the emergency of the event and User preference. When vital sign threshold has been reached, the device 301 will activate alarm logic 315 to stimulate patient and alert bystanders, and alert logic 317 to notify the remote software application which is capable of contacting User-designated contacts. If there is no response from User and/or User-designated contacts, the device 301 will escalate alarms and alerts to User, User-designated Contacts, and eventually contact Emergency Medical Services in attempts to obtain help.

Clinical scenario 1204 illustrates Interventions that the wearable device 301 may initiate if there has been no User response to alarms, device 301 may initiate and escalate noninvasive sensory stimulation to User. Physical stimulation is one of the first actions performed by Emergency Medical Services (EMS) in an unconscious patient, such as a sternal rub. The device 301 may initiate User stimulation such as thoracic vibration, pulsating audible alarms. The device 301 may include additional tactile/haptic stimulation, including rapid pressure changes, thermal (hot/cold) sensations, short electrical stimulation (i.e., via sEMG), and/or any combinations of the above in order to increase User arousal state and improve respiration.

The device 301 may combine sensory stimulation with continued simultaneous notification of caregivers and EMS via audible alarm (for family in the next bedroom, or bystanders), and mobile application alerts and/or phone text/call (for those unable to hear the local alarm, for EMS, etc).

Clinical scenario 1204 also illustrates interventions that the wearable device 301 may initiate if there has been no User response, no User-designated contacts response, and no user response to noninvasive interventions, wearable device 301 is combined with a wearable drug delivery system to provide rapid pharmacologic intervention for seizures. Pharmacologic interventions, reserved for near terminal events, is combined with both sensory stimulation and User and Contacts notification, in some embodiments of the device. Pharmacologic administration can be completed via a wearable drug delivery system that communicates with device 301. Drug delivery device is a device co-located on User with device 301, located on another User body location, or a separate device to be utilized by a caregiver or bystander. Communication is wired, local wireless (Bluetooth, WLAN), or via (User-approved) remote software application.

Components of communication between devices is bidirectional (for communicating vital sign details and intervention response), unidirectional (i.e., drug delivery system cannot access control logic of device 301), or a combination depending on data transmitted.

Clinical scenario 1204 describes pharmacologic interventions that is used when high risk SUDEP event is detected. Epinephrine is a known first rescue medication in cardiopulmonary resuscitation (CPR) and is administered if nonpharmacologic stimulation fails. Serotonin has been shown to minimize the postictal EEG slowing associated with SUDEP and may enter the brain peripherally in seizures due to blood-brain barrier (BBB) leakage. Caffeine is a relatively safe pharmacologic way to stimulate patients when vital signs reach dangerous levels and is already used in pediatric apnea. Theophylline and doxapram may play a role in awakening the patient near SUDEP, as they are currently used to arouse patients post anesthesia.

Clinical scenario 1205 describes pharmacologic interventions that is used when a prolonged seizure is detected (i.e., greater than four minutes long). These seizures are most likely to result in traumatic (hitting objects during seizure), obstructive (face becoming entangled), and centrally mediated complications that may lead to permanent injury and SUDEP. Sedative-hypnotics such as midazolam, diazepam, lorazepam, and others, is used intramuscularly with fast onset and some with a pharmacokinetic profile comparable to intravenous formulations. Said benzodiazepines are the current standard of seizure termination used by Emergency Medical Personnel and Emergency Physicians.

What is claimed:

1. A wearable device for a user comprising
(a) at least one motion-state sensor,
(b) at least one oximetry sensor,
(c) an alarm module configured for transmitting an emergency signal; and
(d) device control logic comprising firmware or embedded software,
wherein the at-least one motion state sensor activates the device control logic upon sensing a no-motion or low-motion state of the user, and the at least one oximetry sensor activates the device control logic during a low-oxygenation or low-respiration state of the user, and
wherein the wearable device continuously monitors for and detects a respiration or oxygenation emergency medical event experienced by the user.

2. The wearable device of claim 1, wherein the device logic activates the at least one oximetry sensor upon receiving information from the at least one motion-state sensor indicating the no-motion or low-motion state of the user.

3. A system comprising the wearable device of claim 1, wherein the receiving device comprises software configured to receive a wireless electronic signal and send a wireless emergency message to pre-designated contacts.

4. The wearable device of claim 1, further comprising a drug delivery device selected from the group consisting of an on-body injector, wearable autoinjector, bolus-injector, patch-pump, and pump for delivery of pharmacological therapeutics to treat an emergency medical event.

5. The wearable device of claim 1, wherein the at least one oximetry sensor consists of two independent oximetry sensors.

6. The wearable device of claim 1, wherein the at least one oximetry sensor comprises:
(a) at least two photo-emitters and at least one photo-receivers in the red spectrum (620-750 nm); and
(b) at least two photo-emitters and at least one photo-receiver in the infrared spectrum (780 nm-1 mm);
wherein the photo-emitters are located 8-10 mm from photo-receivers.

7. The wearable device of claim 1, wherein the alarm module includes at least one vibratory or haptic alarm.

8. The wearable device of claim 1, wherein the alarm module comprises a speaker capable of producing a voice message to engage attention of the user or a bystander, wherein the voice message is selected from the group consisting of identifying an emergency respiratory event; requesting help or emergency medical assistance; providing instruction regarding a response to the emergency medical event; and administrating a rescue.

9. The wearable device of claim 1, wherein the at least one oximetry sensor is configured for bilateral use when worn on complementary right and left sides of the user's body.

10. The wearable device of claim 1, wherein the at least one oximetry sensor probe is in contact with both medial and lateral sides of the limb or torso of the user, or with both anterior and posterior sides of the limb or torso of the user.

11. The wearable device of claim 1, wherein the at least one motion sensor, the at least one oximetry sensor, the alarm module, and the device control logic are distributed along a series of or in a group of segments, wherein the device components are distributed along a series of or in a group of rigid segments, connected by flexible segments containing flexible or wired circuits.

12. The wearable device of claim 11, wherein the sum of the rigid and flexible segments measures 4.0-7.5 inches in length, 0.5-2.0 inches in width and 0.1-0.3 inches in depth.

13. The wearable device of claim 11, wherein the segments are arrayed in an arc measuring 90-270 degrees when worn around the circumference of a limb or torso of the user or wherein the segments are positioned along or in-line at 45-180 degrees to a limb or torso.

14. The wearable device of claim 11, further comprising a physical interface between the electronic components of the device and the skin of the user, wherein the physical interface is configured to conform to an anatomical shape of the user and ensure continuous contact of oximetry probes with the user's skin, while isolating device electronic components from the environment.

15. A method of using the wearable device of claim 1, through continuous operation of a low-power motion-state biosensor, wherein the motion-state biosensor activates the relatively higher-power oximetry sensors following indication of a no motion or low motion state.

16. A method of continuously monitoring the user for a life-threatening respiratory event, using the wearable device of claim 1, comprising the at least one motion-state sensor responding to the no-motion or low-motion state, the at least one oximetry sensor responding to a low-oxygenation or low-respiration state with activation of the alarm module.

17. A method of using the wearable device of claim 1 to prevent a fatal respiratory decline, comprising receiving information of a low-motion state and low-oxygenation or low-respiration states, activating the alarm module, to provoke the user or a bystander to respond.

18. The method of claim 17, further comprising the alarm module triggering a voice message that verbally alerts a bystander to an emergency respiratory event, and provides a request and instructions for responding to emergency respiratory event.

19. The method of claim 17, further comprising triggering a vibratory or haptic alarm to stimulate the patient and improve respiration.

20. The method of claim 17, further comprising engaging a communications module to identify emergency medical event to pre-designated contacts, wherein the pre-designated contacts are selected from the group consisting of user-designated emergency contacts, emergency medical services or community (at-large) responders.
